(19) United States
(12) Patent Application Publication
(10) Pub. No.: US 2025/0011784 A1
(43) Pub. Date: Jan. 9, 2025

(54) RNA MODULATING OLIGONUCLEOTIDES WITH IMPROVED CHARACTERISTICS FOR THE TREATMENT OF NEUROMUSCULAR DISORDERS

(71) Applicant: VICO Therapeutics B.V., Leiden (NL)

(72) Inventors: Peter Christian DE VISSER, Leiden (NL); Susan Allegonda Maria MULDERS, Groesbeek (NL)

(21) Appl. No.: 18/752,689

(22) Filed: Jun. 24, 2024

Related U.S. Application Data
(63) Continuation of application No. 17/728,336, filed on Apr. 25, 2022.
Application No. 17/728,336 is a continuation of application No. 16/660,416, filed on Oct. 22, 2019, now Pat. No. 11,345,915.
Application No. 16/660,416 is a continuation of application No. 15/676,569, filed on Aug. 14, 2017, now abandoned.
Application No. 15/676,569 is a division of application No. 14/522,002, filed on Oct. 23, 2014, now Pat. No. 9,745,576.
Application No. 14/522,002 is a continuation of application No. PCT/NL2013/050306, filed on Apr. 23, 2013.
(60) Provisional application No. 61/636,914, filed on Apr. 23, 2012.

(30) Foreign Application Priority Data
Apr. 23, 2012 (EP) 12165139.2

(51) Int. Cl. C12N 15/113; A61P 25/28

(52) U.S. Cl. CPC C12N 15/113; A61P 25/28; C12N 2310/11; C12N 2310/315; C12N 2310/321; C12N 2310/333; C12N 2310/334; C12N 2310/3341; C12N 2310/335; C12N 2310/336; C12N 2310/346; C12N 2310/3521; C12N 2320/30; C12N 2320/34

(57) ABSTRACT

The current invention provides an improved oligonucleotide and its use for treating, ameliorating, preventing, delaying and/or treating a human cis-clement repeat instability associated genetic neuromuscular or neurodegenerative disorder.

### RELATED APPLICATIONS

This application is a continuation of U.S. patent application Ser. No. 17/728,336, filed Apr. 25, 2022, which is a continuation of U.S. patent application Ser. No. 16/660,416, filed Oct. 22, 2019, now U.S. Pat. No. 11,345,915, which is a continuation of U.S. patent application Ser. No. 15/676,569, filed Aug. 14, 2017, which is a division of U.S. patent application Ser. No. 14/522,002, filed Oct. 23, 2014, now U.S. Pat. No. 9,745,576, which is a continuation of International Patent Application No. PCT/NL2013/050306, filed Apr. 23, 2013, which claims priority to U.S. Patent Application Ser. No. 61/636,914, filed Apr. 23, 2012, and European Patent Application No. 12165139.2, filed Apr. 23, 2012, the disclosures of which are incorporated herein by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML file, created on Oct. 1, 2024, is named 755588_VCO9-002PCCONDIVCON3_ST26.xml and is 697,814 bytes in size.

### FIELD

The invention relates to the field of human genetics, more specifically neuromuscular disorders. The invention in particular relates to the use of antisense oligonucleotides (AONs) with improved characteristics enhancing clinical applicability as further defined herein.

### BACKGROUND OF THE INVENTION

Neuromuscular diseases are characterized by impaired functioning of the muscles due to either muscle or nerve pathology (myopathies and neuropathies). The neuropathies are characterized by neurodegeneration and impaired nerve control leading to problems with movement, spasticity or paralysis. Examples include Huntington's disease (HD), several types of spinocerebellar ataxia (SCA), Friedreich's ataxia (FA), Amyotrophic Lateral Sclerosis (ALS) and Frontotemporal dementia (FTD). A subset of neuropathies is caused by a cis-element repeat instability. For instance, HD is caused by a triplet (CAG) n repeat expansion in exon 1 of the HTT gene. Expansion of these repeats results in expansion of a glutamine stretch at the N-terminal end of the 348 kDa cytoplasmic huntingtin protein. Huntingtin has a characteristic sequence of 6 to 29 glutamine amino acid residues in the normal form; the mutated huntingtin causing the disease has more than 38 residues. The continuous expression of mutant huntingtin molecules in neuronal cells results in the formation of large protein deposits which eventually give rise to cell death, especially in the frontal lobes and the basal ganglia (mainly in the caudate nucleus). The severity of the disease is generally proportional to the number of extra residues. AONs specifically targeting the expanded CAG repeats (such as PS57 (CUG)₇ as a 2′-O-methyl phosphorothioate RNA; SEQ ID NO:1 Evers et al.) can be applied to effectively reduce mutant huntingtin transcript and (toxic) protein levels in HD patient-derived cells. For treatment of neuropathies, systemically administered AONs need to pass the blood brain barrier. Thus, there is a need for optimization of oligochemistry allowing and/or exhibiting improved brain delivery.

The myopathies include genetic muscular dystrophies that are characterized by progressive weakness and degeneration of skeletal, heart and/or smooth muscle. Examples of myopathies are Duchenne muscular dystrophy (DMD), myotonic dystrophy type 1 (DM1), and myotonic dystrophy type 2 (DM2). DM1 and DM2 are both also caused by cis-element repeat instability; DM1 by a trinucleotide (CTG)ₙ repeat expansion in the 3′ untranslated region of exon 15 in the DMPK gene, and DM2 by a tetranucleotide (CCTG)ₙ repeat expansion in the DM2/ZNF9 gene. Also here, AONs specifically targeting the expanded repeats, such as PS58, (CAG)₇, a 2′-O-methyl phosphorothioate RNA for DM1 (Mulders et al.), have been shown to efficiently induce the specific degradation of the (toxic) expanded repeat transcripts. In contrast to DMD where the gene defect is associated with increased permeability of the muscle fiber membranes for small compounds as AONs, for most other myopathies an enhanced AON distribution to and uptake by muscle tissue is essential to obtain a therapeutic effect. Thus, also here there is a need for optimization of oligochemistry allowing and/or exhibiting improved muscle delivery.

The particular characteristics of a chosen chemistry at least in part affect the delivery of an AON to the target transcript: administration route, biostability, biodistribution, intra-tissue distribution, and cellular uptake and trafficking. In addition, further optimization of oligonucleotide chemistry is conceived to enhance binding affinity and stability, enhance activity, improve safety, and/or to reduce cost of goods by reducing length or improving synthesis and/or purification procedures. Multiple chemical modifications have become generally and/or commercially available to the research community (such as 2′-O-methyl RNA and 5-substituted pyrimidines and 2,6-diaminopurines), whereas most others still present significant synthetic effort to obtain. Especially preliminary encouraging results have been obtained using 2′-O-methyl phosphorothioate RNA containing modifications on the pyrimidine and purine bases as identified herein.

In conclusion, to enhance the therapeutic applicability of AONs for treating human cis-element repeat instability associated genetic disorders as exemplified herein, there is a need for AONs with further improved characteristics.

### DESCRIPTION OF THE INVENTION

### Oligonucleotide

In a first aspect, the invention provides an oligonucleotide comprising 2′-O-methyl RNA nucleotide residues, having a backbone wherein at least one phosphate moiety is replaced by a phosphorothioate moiety, and comprising one or more 5-methylpyrimidine and/or one or more 2,6-diaminopurine bases; or an oligonucleotide consisting of 2′-O-methyl RNA nucleotide residues and having a backbone wherein all phosphate moieties are replaced by phosphorothioate moieties, and comprising one or more 5-methylpyrimidine and/or one or more 2,6-diaminopurine bases, for use as a medicament for treating human cis-element repeat instability associated genetic disorders.

In the context of the invention, “backbone” is used to identify the chain of alternating ribose rings and internucleoside linkages, to which the nucleobases are attached. The term “linkage” is used for the connection between two ribose units (i.e. “internucleoside linkage”), which is generally a phosphate moiety. Thus, an oligonucleotide having 10 nucleotides may contain 9 linkages, linking the 10 ribose units together. Additionally, there may be one or more last linkage(s) present at one or both sides of the oligonucleotide, which is only connected to one nucleotide. The terms “linkage” and “internucleoside linkage” are also meant to indicate such a pendant linkage. At least one of the linkages in the backbone of the oligonucleotide according to the invention consists of a phosphorothioate moiety, linking two ribose units. Thus, at least one of the naturally occurring 3′ to 5′ phosphodiester moieties present in RNA is replaced by a phosphorothioate moiety.

Within the context of the invention, “a” in each of the following expressions means “at least one”: a 2′-O-methyl RNA nucleotide residue, a 2′-O-methyl RNA residue, a phosphorothioate moiety, a 2′-O-methyl phosphorothioate RNA residue, a 5-methylpyrimidine base, a 5-methylcytosine base, a 5-methyluracil base, a thymine base, a 2,6-diaminopurine base.

Preferably, the oligonucleotide according to the invention is an oligonucleotide with less than 37 nucleotides. Said oligonucleotide may have 12, 13, 14, 15, 16, 17, 18, 19, 20, 21, 22, 23, 24, 25, 26, 27, 28, 29, 30, 31, 32, 33, 34, 35, or 36 nucleotides. Such oligonucleotide may also be identified as an oligonucleotide having from 12 to 36 nucleotides.

Accordingly, an oligonucleotide of the invention, comprising a 2′-O-methyl RNA nucleotide residue having a backbone wherein at least one phosphate moiety is replaced by a phosphorothioate moiety, comprises less than 37 nucleotides (i.e. it comprises 12, 13, 14, 15, 16, 17, 18, 19, 20, 21, 22, 23, 24, 25, 26, 27, 28, 29, 30, 31, 32, 33, 34, 35 or 36 nucleotides) and a 5-methylpyrimidine and/or a 2,6-diaminopurine base.

Accordingly, an oligonucleotide of the invention, consisting of 2′-O-methyl RNA nucleotide residues and having a backbone wherein all phosphate moieties are replaced by phosphorothioate, and comprises less than 34 nucleotides (i.e. it comprises 12, 13, 14, 15, 16, 17, 18, 19, 20, 21, 22, 23, 24, 25, 26, 27, 28, 29, 30, 31, 32, 33, 34, 35 or 36 nucleotides) and a 5-methylpyrimidine and/or a 2,6-diaminopurine base.

In a preferred embodiment, the oligonucleotide of the invention comprises a 2′-O-methyl phosphorothioate RNA nucleotide residue, or consists of 2′-O-methyl phosphorothioate RNA nucleotide residues. Such oligonucleotide comprises a 2′-O-methyl RNA residue, which is connected through a phosphorothioate linkage to the next nucleotide in the sequence. This next nucleotide may be, but not necessarily, another 2′-O-methyl phosphorothioate RNA nucleotide residue. Alternatively, such oligonucleotide consists of 2′-O-methyl phosphorothioate RNA nucleotide residues, wherein all nucleotides comprise a 2′-O-methyl moiety and a phosphorothioate moiety. Preferably, such oligonucleotide consists of 2′-O-methyl phosphorothioate RNA nucleotide residues. Such chemistry is known to the skilled person. Throughout the application, an oligonucleotide comprising a 2′-O-methyl RNA residue and a phosphorothioate linkage may be replaced by an oligonucleotide comprising a 2′-O-methyl phosphorothioate RNA nucleotide residue or an oligonucleotide comprising a 2′-O-methyl phosphorothioate RNA residue. Throughout the application, an oligonucleotide consisting of 2′-O-methyl RNA residues linked by or connected through phosphorothioate linkages or an oligonucleotide consisting of 2′-O-methyl phosphorothioate RNA nucleotide residues may be replaced by an oligonucleotide consisting of 2′-O-methyl phosphorothioate RNA.

In addition, an oligonucleotide of the invention comprises at least one base modification that increases binding affinity to target strands, increases melting temperature of the resulting duplex of said oligonucleotide with its target, and/or decreases immunostimulatory effects, and/or increases biostability, and/or improves biodistribution and/or intra-tissue distribution, and/or cellular uptake and trafficking. In an embodiment, an oligonucleotide of the invention comprises a 5-methylpyrimidine and/or a 2,6-diaminopurine base. A 5-methylpyrimidine base is selected from a 5-methylcytosine and/or a 5-methyluracil and/or a thymine, in which thymine is identical to 5-methyluracil. Where an oligonucleotide of the invention has two or more such base modifications, said base modifications may be identical, for example all such modified bases in the oligonucleotide are 5-methylcytosine, or said base modifications may be combinations of different base modifications, for example the oligonucleotide may have one or more 5-methylcytosines and one or more 5-methyluracils.

In a preferred embodiment, an oligonucleotide of the invention (i.e. an oligonucleotide comprising 2′-O-methyl RNA nucleotide residues, having a backbone wherein at least one phosphate moiety is replaced by a phosphorothioate moiety, and comprising one or more 5-methylpyrimidine and/or one or more 2,6-diaminopurine bases; or an oligonucleotide consisting of 2′-O-methyl RNA nucleotide residues and having a backbone wherein all phosphate moieties are replaced by phosphorothioate moieties, and comprising one or more 5-methylpyrimidine and/or one or more 2,6-diaminopurine bases) is such that it does not comprise a 2′-deoxy 2′-fluoro nucleotide (i.e. 2′-deoxy 2′-fluoro-adenosine, -guanosine, -uridine and/or -cytidine). Such oligonucleotide comprising a 2′-fluoro (2′-F) nucleotide has been shown to be able to recruit the interleukin enhancer-binding factor 2 and 3 (ILF2/3) and is thereby able to induce exon skipping in the targeted pre-mRNA (Rigo F, et al, WO2011/097614). In the current invention, the oligonucleotide used preferably does not recruit such factors and/or the oligonucleotide of the invention does not form heteroduplexes with RNA that are specifically recognized by the ILF2/3. The mechanism of action of the oligonucleotide of the current invention is assumed to be distinct from the one of an oligonucleotide with a 2′-F nucleotide: the oligonucleotide of the invention is expected to primarily induce the specific degradation of the (toxic) expanded repeat transcripts.

‘Thymine’ and ‘5-methyluracil’ may be interchanged throughout the document. In analogy, 2,6-diaminopurine is identical to 2-aminoadenine and these terms may be interchanged throughout the document.

The term “base modification” or “modified base” as identified herein refers to the modification of a naturally occurring base in RNA (i.e. pyrimidine or purine base) or to the de novo synthesis of a base. This de novo synthesized base could be qualified as “modified” by comparison to an existing base.

An oligonucleotide of the invention comprising a 5-methylcytosine and/or a 5-methyluracil and/or a 2,6-diaminopurine base means that at least one of the cytosine nucleobases of said oligonucleotide has been modified by substitution of the proton at the 5-position of the pyrimidine ring with a methyl group (i.e. a 5-methylcytosine), and/or that at least one of the uracil nucleobases of said oligonucleotide has been modified by substitution of the proton at the 5-position of the pyrimidine ring with a methyl group (i.e. a 5-methyluracil), and/or that at least one of the adenine nucleobases of said oligonucleotide has been modified by substitution of the proton at the 2-position with an amino group (i.e. a 2,6-diaminopurine), respectively. Within the context of the invention, the expression “the substitution of a proton with a methyl group in position 5 of the pyrimidine ring” may be replaced by the expression “the substitution of a pyrimidine with a 5-methylpyrimidine,” with pyrimidine referring to only uracil, only cytosine or both. Likewise, within the context of the invention, the expression “the substitution of a proton with an amino group in position 2 of adenine” may be replaced by the expression “the substitution of an adenine with a 2,6-diaminopurine.” If said oligonucleotide comprises 1, 2, 3, 4, 5, 6, 7, 8, 9 or more cytosines, uracils, and/or adenines, at least 1, 2, 3, 4, 5, 6, 7, 8, 9 or more cytosines, uracils and/or adenines respectively have been modified this way. Preferably all cytosines, uracils and/or adenines have been modified this way or replaced by 5-methylcytosine, 5-methyluracil and/or 2,6-diaminopurine, respectively. No need to say that the invention could only be applied to oligonucleotides comprising at least one cytosine, uracil, or adenine, respectively, in their sequence.

We discovered that the presence of a 5-methylcytosine, 5-methyluracil and/or a 2,6-diaminopurine in an oligonucleotide of the invention has a positive effect on at least one of the parameters or an improvement of at least one parameters of said oligonucleotides. In this context, parameters may include: binding affinity and/or kinetics, silencing activity, biostability, (intra-tissue) distribution, cellular uptake and/or trafficking, and/or immunogenicity of said oligonucleotide, as explained below.

Binding affinity and kinetics depend on the AON's thermodynamic properties. These are at least in part determined by the melting temperature of said oligonucleotide (Tm; calculated with e.g. the oligonucleotide properties calculator (www.unc.edu/˜cail/biotool/oligo/index.html or eu.idtdna.com/analyzer/Applications/OligoAnalyzer/) for single stranded RNA using the basic Tm and the nearest neighbor model), and/or the free energy of the oligonucleotide-target exon complex (using RNA structure version 4.5 or RNA mfold version 3.5). If a Tm is increased, the exon skipping activity typically increases, but when a Tm is too high, the AON is expected to become less sequence-specific. An acceptable Tm and free energy depend on the sequence of the oligonucleotide. Therefore, it is difficult to give preferred ranges for each of these parameters.

An activity of an oligonucleotide of the invention is to inhibit the formation of a mutant protein and/or silence or reduce or decrease the quantity of a disease-associated or disease-causing or mutant transcript containing an extended or unstable number of repeats in a cell of a patient, in a tissue of a patient and/or in a patient as explained later herein. An oligonucleotide of the invention comprising or consisting of a 2′-O-methyl phosphorothioate RNA and a 5-methylcytosine and/or a 5-methyluracil and/or a 2,6-diaminopurine base is expected to be able to silence or reduce or decrease the quantity of said transcript more efficiently than what an oligonucleotide comprising or consisting of a 2′-O-methyl phosphorothioate RNA but without any 5-methylcytosine, without any 5-methyluracil and without any 2,6-diaminopurine base will do. This difference in terms of efficiency may be of at least 1%, 5%, 10%, 15%, 20%, 25%, 30%, 35%, 40%, 45%, 50%, 55%, 60%, 65%, 70%, 75%, 80%, 85%, 90%, 95%, 100%. The reduction or decrease may be assessed by Northern Blotting or (semi-) quantitative RT-PCR for transcript levels (preferably as carried out in the experimental part) or by Western blotting for protein levels. An oligonucleotide of the invention may first be tested in the cellular system like patient-derived fibroblasts as described in Example 1.

Biodistribution and biostability are preferably at least in part determined by a validated hybridization ligation assay adapted from Yu et al., 2002. In an embodiment, plasma or homogenized tissue samples are incubated with a specific capture oligonucleotide probe. After separation, a DIG-labeled oligonucleotide is ligated to the complex and detection followed using an anti-DIG antibody-linked peroxidase. Non-compartmental pharmacokinetic analysis is performed using WINNONLIN software package (model 200, version 5.2, Pharsight, Mountainview, CA). Levels of AON (ug) per mL plasma or mg tissue are monitored over time to assess area under the curve (AUC), peak concentration (Cₘₐₓ), time to peak concentration (Tₘₐₓ), terminal half life and absorption lag time (t_{lag}). Such a preferred assay has been disclosed in the experimental part.

AONs may stimulate an innate immune response by activating the Toll-like receptors (TLR), including TLR9 and TLR7 (Krieg et al., 1995). The activation of TLR9 typically occurs due to the presence of non-methylated CG sequences present in oligodeoxynucleotides (ODNs), by mimicking bacterial DNA which activates the innate immune system through TLR9-mediated cytokine release. The 2′-O-methyl modification is however suggested to markedly reduce such possible effect. TLR7 has been described to recognize uracil repeats in RNA (Diebold et al., 2006).

Activation of TLR9 and TLR7 result in a set of coordinated immune responses that include innate immunity (macrophages, dendritic cells (DC), and NK cells) (Krieg et al., 1995; Krieg, 2000). Several chemo- and cytokines, such as IP-10, TNFα, IL-6, MCP-1 and IFNα (Wagner, 1999; Popovic et al., 2006) have been implicated in this process. The inflammatory cytokines attract additional defensive cells from the blood, such as T and B cells. The levels of these cytokines can be investigated by in vitro testing. In short, human whole blood is incubated with increasing concentrations of AONs after which the levels of the cytokines are determined by standard commercially available ELISA kits. A decrease in immunogenicity preferably corresponds to a detectable decrease of concentration of at least one of the cytokines mentioned above by comparison to the concentration of corresponding cytokine in an assay in a cell treated with an oligonucleotide comprising at least one 5-methylcytosine and/or 5-methyluracil, and/or 2,6-diaminopurine compared to a cell treated with a corresponding oligonucleotide having no 5-methylcytosines, 5-methyluracils, or 2,6-diaminopurines.

Accordingly, a preferred oligonucleotide of the invention has an improved parameter, such as an acceptable or a decreased immunogenicity and/or a better biodistribution and/or acceptable or improved RNA binding kinetics and/or thermodynamic properties by comparison to a corresponding oligonucleotide consisting of a 2′-O-methyl phosphorothioate RNA without a 5-methylcytosine, without a 5-methyluracil and without a 2,6-diaminopurine. Each of these parameters could be assessed using assays known to the skilled person or preferably as disclosed herein.

Below other chemistries and modifications of the oligonucleotide of the invention are defined. These additional chemistries and modifications may be present in combination with the chemistry already defined for said oligonucleotide, i.e. the presence of a 5-methylcytosine, a 5-methyluracil and/or a 2,6-diaminopurine, and the oligonucleotide comprising or consisting of 2′-O-methyl phosphorothioate RNA.

A preferred oligonucleotide of the invention comprises or consists of an RNA molecule or a modified RNA molecule. In a preferred embodiment, an oligonucleotide is single stranded. The skilled person will understand that it is however possible that a single stranded oligonucleotide may form an internal double stranded structure. However, this oligonucleotide is still named a single stranded oligonucleotide in the context of this invention. A single stranded oligonucleotide has several advantages compared to a double stranded siRNA oligonucleotide: (i) its synthesis is expected to be easier than two complementary siRNA strands; (ii) there is a wider range of chemical modifications possible to enhance uptake in cells, a better (physiological) stability and to decrease potential generic adverse effects; (iii) siRNAs have a higher potential for non-specific effects (including off-target genes) and exaggerated pharmacology (e.g. less control possible of effectiveness and selectivity by treatment schedule or dose) and (iv) siRNAs are less likely to act in the nucleus and cannot be directed against introns.

In addition to the modifications described above, the oligonucleotide of the invention may comprise further modifications such as different types of nucleic acid nucleotide residues or nucleotides as described below. Different types of nucleic acid nucleotide residues may be used to generate an oligonucleotide of the invention. Said oligonucleotide may have at least one backbone modification (internucleoside linkage and/or sugar modification) and/or at least one base modification compared to an RNA-based oligonucleotide.

A base modification includes a modified version of the natural purine and pyrimidine bases (e.g. adenine, uracil, guanine, cytosine, and thymine), such as hypoxanthine (e.g. inosine), orotic acid, agmatidine, lysidine, pseudouracil, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N²-cyclopentylguanine (cPent-G), N²-cyclopentyl-2-aminopurine (cPent-AP), and N²-propyl-2-aminopurine (Pr-AP), or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences), which is incorporated here entirely by reference. cPent-G, cPent-AP and Pr-AP were shown to reduce immunostimulatory effects when incorporated in siRNA (Peacock H. et al.).

In an embodiment, an oligonucleotide of the invention comprises an abasic site or an abasic monomer. Within the context of the invention, such monomer may be called an abasic site or an abasic monomer. An abasic monomer or abasic site is a nucleotide residue or building block that lacks a nucleobase by comparison to a corresponding nucleotide residue comprising a nucleobase. Within the invention, an abasic monomer is thus a building block part of an oligonucleotide but lacking a nucleobase. Such abasic monomer may be present or linked or attached or conjugated to a free terminus of an oligonucleotide.

In a more preferred embodiment, an oligonucleotide of the invention comprises 1-10 or more abasic monomers. Therefore, 1, 2, 3, 4, 5, 6, 7, 8, 9, 10 or more abasic monomers may be present in an oligonucleotide of the invention.

An abasic monomer may be of any type known and conceivable by the skilled person, non-limiting examples of which are depicted below:

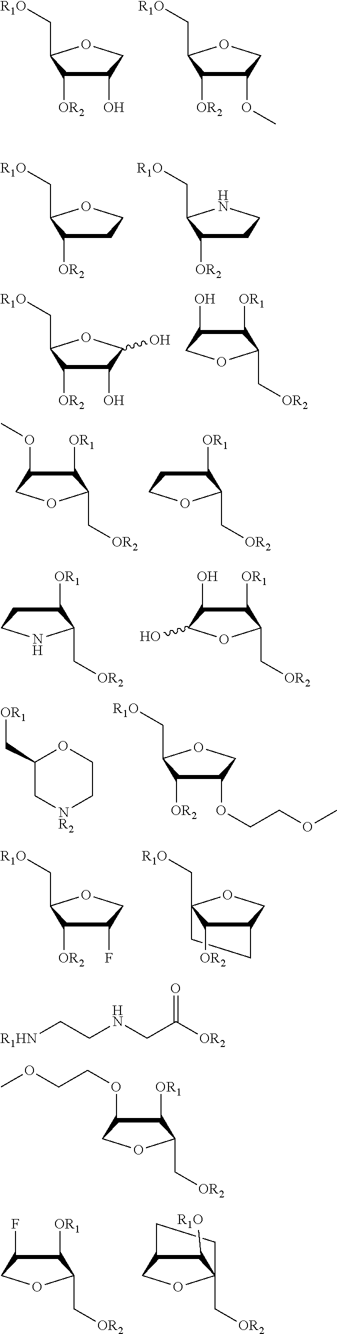

Herein, R₁ and R₂ are independently H, an oligonucleotide or other abasic site(s), provided that not both R₁ and R₂ are H and R₁ and R₂ are not both an oligonucleotide. An abasic monomer(s) can be attached to either or both termini of the oligonucleotide as specified before. It should be noted that an oligonucleotide attached to one or two an abasic site(s) or abasic monomer(s) may comprise less than 12 nucleotides. In this respect, the oligonucleotide according to the invention may comprise at least 12 nucleotides, optionally including one or more abasic sites or abasic monomers at one or both termini.

In the sequence listing, an oligonucleotide of the invention comprising an abasic monomer may be represented by its nucleotide or base sequence; the abasic monomer not being represented since it may be considered as linked or attached or conjugated to a free terminus of an oligonucleotide. This is the case for base sequences SEQ ID NO:107 and 108. In table 2, the full sequence of preferred oligonucleotides comprising SEQ ID NO:107 or 108 is provided: such oligonucleotide comprises SEQ ID NO:107 or 108 and 4 abasic monomers at the 3′ terminus of the corresponding SEQ ID NO:107 or 108. SEQ ID NO:220 and 221 correspond to SEQ ID NO:107 and 108 further comprising 4 additional abasic monomers at the 3′ terminus of the oligonucleotide.

When an abasic monomer is present within a base sequence of an oligonucleotide, said abasic monomer is identified in the sequence listing as part of the sequence of said oligonucleotide as in SEQ ID NO:210 and 213.

In tables 1 and 2, an abasic monomer is identified using the letter Q.

Depending on its length an oligonucleotide of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 base modifications. It is also encompassed by the invention to introduce more than one distinct base modification in said oligonucleotide.

A “sugar modification” indicates the presence of a modified version of the ribosyl moiety as naturally occurring in RNA (i.e. the furanosyl moiety), such as bicyclic sugars, tetrahydropyrans, morpholinos, 2′-modified sugars, 4′-modified sugars, 5′-modified sugars, and 4′-subsituted sugars. Examples of suitable sugar modifications include, but are not limited to, 2′-O-modified RNA nucleotide residues, such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-allyl, 2′-O-(2-amino)propyl, 2′-O-(2-(dimethylamino)propyl), 2′-O-(2-amino)ethyl, 2′-O-(2-(dimethylamino)ethyl); 2′-deoxy (DNA); 2′-O-(haloalkoxy)methyl (Arai K. et al.) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl] (MOCE), 2′-O-[2-(N-methylcarbamoyl)ethyl] (MCE), 2′-O-[2-(N,N-dimethylcarbamoyl)ethyl] (DCME); 2′-halo e.g. 2′-F, FANA (2′-F arabinosyl nucleic acid); carbasugar and azasugar modifications; 3′-O-alkyl e.g. 3′-O-methyl, 3′-O-butyryl, 3′-O-propargyl, 5′-alkyl e.g. 5′-methyl; and their derivatives.

Another sugar modification includes “bridged” or “bicylic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), BNA^{NC}[N-Me] (as described in Chem. Commun. 2007, 3765, which is incorporated in its entirety by reference); tricyclo DNA (tcDNA); unlocked nucleic acid (UNA); 5′-methyl substituted BNAs (as described in U.S. patent application Ser. No. 13/530,218, which is incorporated in its entirety by reference); cyclohexenyl nucleic acid (CeNA), altriol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PMOPlus, PMO-X) and their derivatives, preferably locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), tricyclo DNA (tcDNA); cyclohexenyl nucleic acid (CeNA), altriol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PMOPlus, PMO-X) and their derivatives. A preferred tcDNA is tc-PS-DNA (tricyclo DNA comprising phosphorothioate internucleoside linkage). Depending on its length, an oligonucleotide of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 sugar modifications. It is also encompassed by the invention to introduce more than one distinct sugar modification in said oligonucleotide. In an embodiment, an oligonucleotide as defined herein comprises or consists of an LNA or a derivative thereof. BNA derivatives are for example described in WO 2011/097641, which is incorporated in its entirety by reference. In a more preferred embodiment, an oligonucleotide of the invention is fully 2′-O-methyl modified. Examples of PMO-X are described in WO2011150408, which is incorporated here in its entirety by reference. In a preferred embodiment, the oligonucleotide according to the invention comprises, apart from the mandatory 2′-O-methyl sugar modification, at least one other sugar modification selected from 2′-O-methyl, 2′-O-(2-methoxy)ethyl, morpholino, a bridged nucleotide or BNA, or the oligonucleotide comprises both bridged nucleotides and 2′-deoxy modified nucleotides (BNA/DNA mixmers). More preferably, the oligonucleotide according to the invention is modified over its full length with a sugar modification selected from 2′-O-methyl, 2′-O-(2-methoxy)ethyl, morpholino, bridged nucleic acid (BNA) or BNA/DNA mixmer.

In a more preferred embodiment, the oligonucleotide according to the invention comprises is fully 2′-O-methyl modified, preferably fully 2′-O-methyl phosphorothioate modified.

A “backbone modification” indicates the presence of a modified version of the ribosyl moiety (“sugar modification”), as indicated above, and/or the presence of a modified version of the phosphodiester as naturally occurring in RNA (“internucleoside linkage modification”). Examples of internucleoside linkage modifications, which are compatible with the present invention, are phosphorothioate (PS), chirally pure phosphorothioate, phosphorodithioate (PS2), phosphonoacetate (PACE), phosphonoacetamide (PACA), thiophosphonoacetate, thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, and their derivatives. Another modification includes phosphoramidite, phosphoramidate, N3′→P5′ phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives. Depending on its length, an oligonucleotide of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 backbone modifications. It is also encompassed by the invention to introduce more than one distinct backbone modification in said oligonucleotide.

An oligonucleotide of the invention comprises at least one phosphorothioate modification. In a more preferred embodiment, an oligonucleotide of the invention is fully phosphorothioate modified.

Other chemical modifications of an oligonucleotide of the invention include peptide-base nucleic acid (PNA), boron-cluster modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), morpholino-based oligonucleotide (PMO, PPMO, PMO-X), cationic morpholino-based oligomers (PMOPlus), oligonucleotides with integrated bases and backbones (ONIBs), pyrrolidine-amide oligonucleotides (POMs); and their derivatives.

In another embodiment, an oligonucleotide comprises a peptide nucleic acid and/or a morpholino phosphorodiamidate or a derivative thereof.

Thus, the preferred oligonucleotide according to one aspect of the invention comprises:
(a) at least one base modification selected from 5-methylpyrimidine and 2,6-diaminopurine; and/or
(b) at least one sugar modification, which is 2′-O-methyl, and/or
(c) at least one backbone modification, which is phosphorothioate.

Thus, a preferred oligonucleotide according to this aspect of the invention comprises a base modification (a) and no sugar modification (b) and no backbone modification (c). Another preferred oligonucleotide according to this aspect of the invention comprises a sugar modification (b) and no base modification (a) and no backbone modification (c). Another preferred oligonucleotide according to this aspect of the invention comprises a backbone modification (c) and no base modification (a) and no sugar modification (b). Also oligonucleotides having none of the above-mentioned modifications are understood to be covered by the present invention, as well as oligonucleotides comprising two, i.e. (a) and (b), (a) and (c) and/or (b) and (c), or all three of the modifications (a), (b) and (c), as defined above. In another preferred embodiment, any of the oligonucleotides as described in the previous paragraph may comprise:
(a) at least one (additional) base modification selected from 2-thiouracil, 2-thiothymine, 5-methylcytosine, 5-methyluracil, thymine, 2,6-diaminopurine; and/or
(b) at least one (additional) sugar modification selected from 2′-O-methyl, 2′-O-(2-methoxy)ethyl, 2′-deoxy (DNA), morpholino, a bridged nucleotide or BNA, or the oligonucleotide comprises both bridged nucleotides and 2′-deoxy modified nucleotides (BNA/DNA mixmers); and/or
(c) at least one (additional) backbone modification selected from (another) phosphorothioate or phosphordiamidate.

In another preferred embodiment, the oligonucleotide according to the invention is modified over its entire length with one or more of the same modification, selected from (a) one of the base modifications; and/or (b) one of the sugar modifications; and/or (c) one of the backbone modifications.

With the advent of nucleic acid mimicking technology, it has become possible to generate molecules that have a similar, preferably the same hybridization characteristics in kind not necessarily in amount as nucleic acid itself. Such functional equivalents are of course also suitable for use in the invention.

The skilled person will understand that not each sugar, base, and/or backbone may be modified the same way. Several distinct modified sugars, bases and/or backbones may be combined into one single oligonucleotide of the invention.

A person skilled in the art will also recognize that there are many synthetic derivatives of oligonucleotides.

Preferably, said oligonucleotide comprises RNA, as RNA/RNA duplexes are very stable. It is preferred that an RNA oligonucleotide comprises a modification providing the RNA with an additional property, for instance resistance to endonucleases, exonucleases, and RNaseH, additional hybridisation strength, increased stability (for instance in a bodily fluid), increased or decreased flexibility, increased activity, reduced toxicity, increased intracellular transport, tissue-specificity, etc. In addition, the mRNA complexed with the oligonucleotide of the invention is preferably not susceptible to RNaseH cleavage. Preferred modifications have been identified above.

Oligonucleotides containing at least in part naturally occurring DNA nucleotides are useful for inducing degradation of DNA-RNA hybrid molecules in the cell by RNase H activity (EC.3.1.26.4).

Naturally occurring RNA ribonucleotides or RNA-like synthetic ribonucleotides comprising oligonucleotides are encompassed herein to form double stranded RNA-RNA hybrids that act as enzyme-dependent antisense through the RNA interference or silencing (RNAi/siRNA) pathways, involving target RNA recognition through sense-antisense strand pairing followed by target RNA degradation by the RNA-induced silencing complex (RISC).

Alternatively or in addition, an oligonucleotide can interfere with the processing or expression of precursor RNA or messenger RNA (steric blocking, RNaseH independent processes) in particular but not limited to RNA splicing and exon skipping, by binding to a target sequence of RNA transcript and getting in the way of processes such as translation or blocking of splice donor or splice acceptor sites. Moreover, the oligonucleotide may inhibit the binding of proteins, nuclear factors and others by steric hindrance and/or interfere with the authentic spatial folding of the target RNA and/or bind itself to proteins that originally bind to the target RNA and/or have other effects on the target RNA, thereby contributing to the destabilization of the target RNA, preferably pre-mRNA, and/or to the decrease in amount of diseased or toxic transcript and/or protein in diseases like HD as identified later herein.

As herein defined, an oligonucleotide may comprise nucleotides with (RNaseH resistant) chemical substitutions at least one of its 5′ or 3′ ends, to provide intracellular stability, and comprises less than 9, more preferably less than 6 consecutive (RNaseH-sensitive) deoxyribose nucleotides in the rest of its sequence. The rest of the sequence is preferably the center of the sequence. Such oligonucleotide is called a gapmer. Gapmers have been extensively described in WO 2007/089611. Gapmers are designed to enable the recruitment and/or activation of RNaseH. Without wishing to be bound by theory, it is believed that RNaseH is recruited and/or activated via binding to the central region of the gapmer made of deoxyriboses. An oligonucleotide of the invention which is preferably substantially independent of or independent of RNaseH is designed in order to have a central region which is substantially not able or is not able to recruit and/or activate RNaseH. In a preferred embodiment, the rest of the sequence of said oligonucleotide, more preferably its central part comprises less than 9, 8, 7, 6, 5, 4, 3, 2, 1, or no deoxyribose. Accordingly, this oligonucleotide of the invention is preferably partly to fully replaced as earlier defined herein. “Partly replaced” means that the oligonucleotide comprises at least some of nucleotides that have been replaced, preferably at least 50% of its nucleotides have been replaced, or at least 55%, 60%, 65%, 70%, 75%, 80%, 85%, 90% or 95% have been replaced. 100% replacement of nucleotides corresponds to “fully replaced”.

Accordingly, the invention provides an oligonucleotide comprising a 2′-O-methyl phosphorothioate RNA residue or consisting of 2′-O-methyl phosphorothioate RNA and comprising a 5-methylpyrimidine and/or a 2,6-diaminopurine base. Most preferably, this oligonucleotide consists of 2′-O-methyl RNA residues connected through a phosphorothioate backbone and all of its cytosines and/or all of its uracils and/or all of its adenines, independently, have been replaced by 5-methylcytosine, 5-methyluracil and/or 2,6-diaminopurine, respectively. Thus, an oligonucleotide of the invention may have:
At least one and preferably all cytosines replaced with 5-methylcytosines,
At least one and preferably all cytosines replaced with 5-methylcytosines and at least one and preferably all uracils replaced with 5-methyluracils,
At least one and preferably all cytosines replaced with 5-methylcytosines and at least one and preferably all adenines replaced with 2,6-diaminopurines,
At least one and preferably all cytosines replaced with 5-methylcytosines and at least one and preferably all uracils replaced with 5-methyluracils and at least one and preferably all adenines replaced with 2,6-diaminopurines,
At least one and preferably all uracils replaced with 5-methyluracils,
At least one and preferably all uracils replaced with 5-methyluracils and at least one and preferably all adenines replaced with 2,6-diaminopurines, or
At least one and preferably all adenines replaced with 2,6-diaminopurines.

An oligonucleotide of the invention is for use as a medicament for preventing delaying and/or treating a human cis-element repeat instability associated genetic disorders preferably as exemplified herein. A human cis-element repeat instability associated genetic disorders as identified herein is preferably a neuromuscular disorder. Preferably said oligonucleotide is for use in therapeutic RNA modulation. Therefore, the oligonucleotide according to the invention may be described as an antisense oligonucleotide (AON). An antisense oligonucleotide is an oligonucleotide which binds (or is able to bind), targets, hybridizes to (or is able to hybridize to) and/or is reverse complementary to a specific sequence of a transcript of a gene which is known to be associated with or involved in a human cis-element repeat instability associated genetic neuromuscular disorder.

According to the invention, an antisense oligonucleotide comprising or consisting of 2′-O-methyl RNA nucleotide residues, having a backbone wherein at least one phosphate moiety is replaced by a phosphorothioate moiety, and further comprising at least one of a 5-methylcytosine and/or a 5-methyluracil and/or a 2,6-diaminopurine, is represented by a nucleotide sequence comprising or consisting of a sequence that binds (or is able to bind), hybridizes (or is able to hybridize), targets and/or is reverse complementary to a repetitive element in a RNA transcript having as repetitive nucleotide unit a repetitive nucleotide unit, which is selected from the (CAG)ₙ, (GCG)ₙ, (CGG)ₙ, (GAA)ₙ, (GCC)ₙ, (CCG)ₙ, (AUUCU)ₙ, (GGGGCC)ₙ or (CCUG)ₙ. Said oligonucleotide is preferably a single stranded oligonucleotide.

Although it is to be understood that an oligonucleotide of the invention binds (or is able to bind), hybridizes (or is able to hybridize), targets and/or is reverse complementary to a repetitive element present in a RNA transcript as identified above, it can not be ruled out that such oligonucleotide may also interfere with or bind (or is able to bind) or hybridize to (or is able to hybridize) a corresponding DNA, this RNA transcript is derived from.

A repeat or repetitive element or repetitive sequence or repetitive stretch is herein defined as a repetition of at least 3, 4, 5, 10, 100, 1000 or more, of a repetitive unit or repetitive nucleotide unit or repeat nucleotide unit (as (CAG)ₙ, (GCG)ₙ, (CGG)ₙ, (GAA)ₙ, (GCC)ₙ, (CCG)ₙ, (AUUCU)ₙ, (GGGGCC)ₙ or (CCUG)ₙ), comprising a trinucleotide repetitive unit, or alternatively a 4, 5 or 6 nucleotide repetitive unit, in a transcribed gene sequence in the genome of a subject, including a human subject. Accordingly, n is an integer and may be at least 3, 4, 5, 10, 100, 1000 or more. The invention is not limited to exemplified repetitive nucleotide units. Other repetitive nucleotide unit could be found on the following site neuromuscular.wustl.edu/mother/dnarep.htm. In the majority of patients, a “pure” repeat or repetitive element or repetitive sequence or repetitive stretch as identified above (as (CAG)ₙ, (GCG)ₙ, (CGG)ₙ, (GAA)ₙ, (GCC)ₙ, (CCG)ₙ, (AUUCU)ₙ, (GGGGCC)ₙ or (CCUG)ₙ) is present in a transcribed gene sequence in the genome of said patient. However, it is also encompassed by the invention, that in some patients, said repeat or repetitive element or repetitive sequence or repetitive stretch as identified above is not qualified as “pure” or is qualified as a “variant” when for example said repeat or repetitive element or repetitive sequence or repetitive stretch as identified above is interspersed with at least 1, 2, or 3 nucleotide(s) that do not fit the nucleotide(s) of said repeat or repetitive element or repetitive sequence or repetitive stretch (Braida C., et al,).

An oligonucleotide according to the invention therefore may not need to be 100% reverse complementary to a targeted repeat. Usually an oligonucleotide of the invention may be at least 90%, 95%, 97%, 99% or 100% reverse complementary to a targeted repeat.

In an embodiment, an antisense oligonucleotide comprises or consists of 2′-O-methyl phosphorothioate RNA, comprises a 5-methylcytosine and/or a 5-methyluracil and/or a 2,6-diaminopurine, is represented by a nucleotide sequence comprising or consisting of a sequence that binds (or is able to bind), hybridizes (or is able to hybridize), targets and/or is reverse complementary to a (CAG)ₙ tract in a transcript and is particularly useful for the treatment, delay, amelioration and/or prevention of the human genetic diseases Huntington's disease (HD), spinocerebellar ataxia (SCA) type 1, 2, 3, 6, 7, 12 or 17, amyotrophic lateral sclerosis (ALS), frontotemporal dementia (FTD), X-linked spinal and bulbar muscular atrophy (SBMA) and/or dentatorubropallidoluysian atrophy (DRPLA) caused by CAG repeat expansions in the transcripts of the HTT (SEQ ID NO:80), ATXN1 (SEQ ID NO:81), ATXN2 (SEQ ID NO:82) ATXN3 (SEQ ID NO:83), CACNA1A (SEQ ID NO:84), ATXN7 (SEQ ID NO:85), PPP2R2B (SEQ ID NO:86), TBP (SEQ ID NO:87), AR (SEQ ID NO:88) or ATN1 (SEQ ID NO:89) genes. Preferably, these genes are from human origin. In this embodiment, an oligonucleotide comprises or consists of 2′-O-methyl phosphorothioate RNA, comprises a 5-methylcytosine and/or a 5-methyluracil and/or a 2,6-diaminopurine, is represented by a nucleotide sequence comprising or consisting of a sequence that binds (or is able to bind), hybridizes (or is able to hybridize), targets and/or is reverse complementary to a (CAG)ₙ repeat as identified above and has as repetitive nucleotide unit (CUG)ₘ. The m in (CUG)ₘ is preferably an integer which is 4, 5, 6, 7, 8, 9, 10, 11, 12. In a preferred embodiment, m is 5 or 6 or 7 or 8 or 9 or 10 or 11 or 12.

It is to be noted that for ALS and FTD, it is known that at least two distinct repeats in at least two distinct transcripts may be involved or may be responsible or linked with the disease. One has been identified in the previous paragraph (i.e. (CAG)ₙ in a ATXN2 transcript). Another one is being identified later as a (GGGGCC)ₙ repeat or tract in a C9ORF72 transcript. It means that for each of these two diseases, one may envisage to use either one of these two distinct oligonucleotides of the invention to specifically induce the specific degradation of the corresponding (toxic) expanded repeat transcripts.

Throughout the application, an oligonucleotide defined as being reverse complementary to, binding (being able to bind), hybridizing (being able to hybridize) or targeting a repeat as identified above and has or comprises a repetitive nucleotide unit may have any length comprised from 12 to 36 nucleotides. If we take the example of CUG as repetitive nucleotide unit comprised within said oligonucleotide, any oligonucleotide comprising UGC or GCU as repetitive nucleotide unit is also encompassed by the present invention. Depending on the length of said oligonucleotide (for example from 12 to 36 nucleotides), the given repetitive nucleotide unit may not be complete at the 5′ and/or at the 3′ side of said oligonucleotide. Each of said oligonucleotide is encompassed within the scope of said invention.

Alternatively, if we still take as an example the oligonucleotide having CUG as repetitive nucleotide unit, it may be represented by H—(P)ₚ—(CUG)ₘ—(Q)_{q}—H, wherein m is an integer as defined above. Each occurrence of P and Q is, individually, an abasic monomer as defined above or a nucleotide, such as A, C, G, U or an analogue or equivalent thereof and p and q are each individually an integer, preferably 0, 1, 2, 3, 4, 5, 6, 7, 8, 9, 10, 11, 12, 13, 14, 15, 16, 17, 18, 19, 20 or higher up to 100. Thus, p and q are each individually an integer from 0 to 100, preferably an integer from 0 to 20, more preferably an integer from 0 to 10, more preferably from 0 to 6, even more preferably from 0 to 3. Thus, when p is 0, P is absent and when q is 0, Q is absent. The skilled person will appreciate that an oligonucleotide will always start with and end with a hydrogen atom (H), regardless of the amount and nature of the nucleotides present in the oligonucleotide.

It will be appreciated that herein (CUG)ₘ may be replaced by any repeating nucleotide unit within the context of the invention. Thus, a preferred oligonucleotide according to the invention may be represented by H—(P)ₚ—(R)ᵣ—(Q)_{q}—H, wherein (R)ᵣ is a repeating nucleotide unit within the context of the invention and P, Q, p and q are as defined above.

In the context of the present invention, an “analogue” or an “equivalent” of a nucleotide is to be understood as a nucleotide which comprises at least one modification with respect to the nucleotides naturally occurring in RNA, such as A, C, G and U. Such a modification may be a internucleoside linkage modification and/or a sugar modification and/or a base modification, as explained and exemplified above.

Again taking the oligonucleotide having CUG as repetitive nucleotide unit, it is to be understood that the repeating sequence may start with either a C, a U or a G. Thus, in a preferred embodiment, p is not 0, and (P)ₚ is represented by (P′)_{p′}UG or (P′)_{p″}G, wherein each occurrence of P′ is, individually, an abasic site or a nucleotide, such as A, C, G, U or an analogue or equivalent thereof, and p′ is p−2 and p″ is p−1. Such oligonucleotides may be represented as:

H—(P′)_{p′}UG—(CUG)ₘ—(Q)_{q}—H or

H—(P′)_{p″}G—(CUG)ₘ—(Q)_{q}—H.

In an equally preferred embodiment, q is not 0, and (Q)_{q} is represented by CU(Q′)_{q′} or C(Q′)_{q″} and each occurrence of Q′ is, individually, an abasic site or a nucleotide, such as A, C, G, U or an analogue or equivalent thereof, and q′ is q−2 and q″ is q−1. Such oligonucleotides may be represented as:

H—(P′)ₚ—(CUG)ₘ—CU(Q′)_{q′}—H or

H—(P)ₚ—(CUG)ₘ—C(Q′)_{q″}—H.

In another preferred embodiment, both p and q are not 0, and both (P)ₚ and (Q)_{q} are represented by (P′)_{p′}UG or (P′)_{p″}G and CU(Q′)_{q} or C(Q′)_{q″} respectively, wherein P′, Q′, p′, p″, q′ and q″ are as defined above. Such oligonucleotides may be represented as:

H—(P′)_{p′}UG—(CUG)ₘ—CU(Q′)_{q′}—H,

H—(P′)_{p″}G—(CUG)ₘ—CU(Q′)_{q′}—H,

H—(P′)_{p′}UG—(CUG)ₘ—C(Q′)_{q″}—H, or

H—(P′)_{p″}G—(CUG)ₘ—C(Q′)_{q″}—H.

It is to be understood that p′, p″, q′ and q″ may not be negative integers. Thus, when (P)ₚ is represented by (P′)_{p′} UG or (P′)_{p″}G, p is at least 1 or at least 2 respectively, and when (Q)_{q} is represented by CU(Q′)_{q′} or C(Q′)_{q″}, q is at least 1 or at least 2 respectively. It is to be understood that all said here regarding the CUG repeat unit can be extended to any repeat unit within the context of the invention.

In a preferred embodiment, an oligonucleotide defined as being reverse complementary to, binding (or being able to bind), hybridizing (or being able to hybridize) or targeting a (CAG)ₙ repeat comprises or consists of a repetitive nucleotide unit (XYG)ₘ and has a length comprised from 12 to 36 nucleotides and wherein each X is C or 5-methylcytosine, and each Y is U or 5-methyluracil such that at least one X is 5-methylcytosine and/or at least one Y is 5-methyluracil.m is an integer. In the context of this embodiment, m may be 4, 5, 6, 7, 8, 9, 10, 11, 12. A preferred value for m is 7.

A more preferred oligonucleotide therefore comprises or consists of a repetitive nucleotide unit (XYG)ₘ, wherein each X is C or 5-methylcytosine, and each Y is U or 5-methyluracil such that at least one X is 5-methylcytosine and/or at least one Y is 5-methyluracil, and m is an integer from 4 to 12 (SEQ ID NO:2 to 12).

An even more preferred oligonucleotide comprises or consists of a repetitive nucleotide unit (XYG)ₘ, wherein each X is 5-methylcytosine, and/or each Y is 5-methyluracil, and m is an integer from 4 to 12 (SEQ ID NO:2 to 12).

An even more preferred oligonucleotide therefore comprises or consists of a repetitive nucleotide unit (XYG)₅, (XYG)₆ or (XYG)₇, (XYG)₈, or (XYG)₉ wherein each X is C or 5-methylcytosine, and each Y is U or 5-methyluracil such that at least one X is 5-methylcytosine and/or at least one Y is 5-methyluracil. More preferred is an oligonucleotide comprising or consisting of (XYG)₇, wherein each X is C or 5-methylcytosine, and each Y is U or 5-methyluracil such that at least one X is 5-methylcytosine and/or at least one Y is 5-methyluracil (SEQ ID NO:7).

An even more preferred oligonucleotide comprises or consists of a repetitive nucleotide unit (XYG)₇, wherein each X is 5-methylcytosine and each Y is a uracil (SEQ ID NO:2), or each X is a cytosine and each Y is 5-methyluracil (SEQ ID NO:3). An even more preferred oligonucleotide comprises SEQ ID NO:2 or 3 and has a length of 21, 22, 23, 24, 25, 26, 27, 28, 29, 30 nucleotides.

Most preferred oligonucleotides sequences comprising or consisting of a repetitive nucleotide unit (XYG)ₘ have been identified in table 2 as SEQ ID NO:90-118.

A preferred oligonucleotide comprising or consisting of 2′-O-methyl phosphorothioate RNA comprises one of base sequences SEQ ID NO:90-106 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:90-106 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:90-106 and has a length of 21 nucleotides.

A preferred oligonucleotide comprising or consisting of 2′-O-methyl phosphorothioate RNA comprises one of base sequences SEQ ID NO:107 or 108 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:107 or 108 and has a length from 21-36 nucleotides, more preferably 21, 22, 23, 24, 25, 26, 27, 28, 29, 30, 31, 32, 33, 34, 35 or 36 nucleotides. Even more preferred oligonucleotide consists of 2′-O-methyl phosphorothioate RNA, has a base sequence that consists of one of the base sequences SEQ ID NO:107 or 108 and has a length of 21 nucleotides. Most preferred oligonucleotide consists of 2′-O-methyl phosphorothioate RNA, has a base sequence that consists of one of the base sequences SEQ ID NO:107 or 108, has a length of 21 nucleotides and additionally comprises 4 abasic monomers at one of its termini, preferably at the 3′ terminus. Said most preferred oligonucleotide is represented by a base sequence consisting of SEQ ID NO:220 or 221.

A preferred oligonucleotide comprising or consisting of 2′-O-methyl phosphorothioate RNA comprises one of base sequences SEQ ID NO:109 or 110 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:109 or 110 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:109 or 110 and has a length of 24 nucleotides.

A preferred oligonucleotide comprising or consisting of 2′-O-methyl phosphorothioate RNA comprises one of base sequences SEQ ID NO:111 or 112 and has a length from 27-36 nucleotides, more preferably 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:111 or 112 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:111 or 112 and has a length of 27 nucleotides.

A preferred oligonucleotide comprising or consisting of 2′-O-methyl phosphorothioate RNA comprises one of base sequences SEQ ID NO:113 or 114 and has a length from 30-36 nucleotides, more preferably 30, 31, 32, 33, 34, 35 or 36 nucleotides. An even more preferred oligonucleotide consists of 2′-O-methyl phosphorothioate RNA and comprises one of the base sequences SEQ ID NO:113 or 114 and has a length from 30-36 nucleotides, more preferably 30, 31, 32, 33, 34, 35 or 36 nucleotides. Most preferred oligonucleotide consists of 2′-O-methyl phosphorothioate RNA, has a base sequence that consists of one of the base sequences SEQ ID NO:113 or 114 and has a length of 30 nucleotides.

A preferred oligonucleotide comprising or consisting of 2′-O-methyl phosphorothioate RNA comprises one of base sequences SEQ ID NO:115 or 116 and has a length from 33-36 nucleotides, more preferably 33, 34, 35 or 36 nucleotides. An even more preferred oligonucleotide consists of 2′-O-methyl phosphorothioate RNA and comprises one of the base sequences SEQ ID NO:115 or 116 and has a length from 33-36 nucleotides, more preferably 33, 34, 35 or 36 nucleotides. Most preferred oligonucleotide consists of 2′-O-methyl phosphorothioate RNA, has a base sequence that consists of one of the base sequences SEQ ID NO:115 or 116 and has a length of 33 nucleotides.

A preferred oligonucleotide comprising or consisting of 2′-O-methyl phosphorothioate RNA comprises one of base sequences SEQ ID NO:117 or 118 and has a length of 36 nucleotides. Most preferred oligonucleotide consists of 2′-O-methyl phosphorothioate RNA, has a base sequence that consists of one of the base sequences SEQ ID NO:117 or 118 and has a length of 36 nucleotides.

In another embodiment, an antisense oligonucleotide comprising or consisting of 2′-O-methyl phosphorothioate RNA, and comprising a 5-methylcytosine is represented by a nucleotide sequence comprising or consisting of a sequence that binds to (or is able to bind to), hybridizes (or is able to hybridize), targets and/or is reverse complementary to a (GCG)ₙ repeat in a transcript and is particularly useful for the treatment, delay, amelioration and/or prevention of the human genetic diseases: infantile spasm syndrome, deidocranial dysplasia, blepharophimosis, hand-foot-genital disease, synpolydactyly, oculopharyngeal muscular dystrophy and/or holoprosencephaly, which are caused by repeat expansions in the ARX, CBFA1, FOXL2, HOXA13, HOXD13, OPDM/PABP2, TCFBR1 or ZIC2 genes. Preferably, these genes are from human origin.

In a preferred embodiment, an oligonucleotide defined as being reverse complementary to, binding (or being able to bind), hybridizing (or being able to hybridize) or targeting a (GCG)ₙ repeat comprises or consists of a repetitive nucleotide unit (XGX)ₘ and has a length comprised from 12 to 36 nucleotides and wherein each X is C or 5-methylcytosine, such that at least one X is 5-methylcytosine.m is an integer. In the context of this embodiment, m may be 4, 5, 6, 7, 8, 9, 10, 11, 12. A preferred value for m is 7.

A more preferred oligonucleotide therefore comprises or consists of a repetitive nucleotide unit (XGX)ₘ, wherein at least one X is 5-methylcytosine, and m is an integer from 4 to 12 (SEQ ID NO:13 to 21). An even more preferred oligonucleotide comprises or consists of a repetitive nucleotide unit (XGX)ₘ, wherein each X is 5-methylcytosine, and m is an integer from 4 to 12 (SEQ ID NO:13 to 21).

An even more preferred oligonucleotide therefore comprises or consists of a repetitive nucleotide unit (XGX)₇ (SEQ ID NO:16), wherein at least one X is 5-methylcytosine.

An even more preferred oligonucleotide comprises or consists of a repetitive nucleotide unit (XGX)₇ (SEQ ID NO:16), wherein each X is 5-methylcytosine.

Most preferred oligonucleotides sequences comprising or consisting of a repetitive nucleotide unit (XGX)ₘ have been identified in table 2 as SEQ ID NO:119-132.

A preferred oligonucleotide comprising or consisting of 2′-O-methyl phosphorothioate RNA comprises one of base sequences SEQ ID NO:119 or 120 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:119 or 120 and has a length from 16-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:119 or 120 and has a length of 12 nucleotides.

A preferred oligonucleotide comprising or consisting of 2′-O-methyl phosphorothioate RNA comprises one of base sequences SEQ ID NO:121 or 122 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:90-106 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:121 or 122 and has a length of 15 nucleotides.

A preferred oligonucleotide comprising or consisting of 2′-O-methyl phosphorothioate RNA comprises one of base sequences SEQ ID NO:123 or 124 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:123 or 124 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:123 or 124 and has a length of 18 nucleotides.

A preferred oligonucleotide comprising or consisting of 2′-O-methyl phosphorothioate RNA comprises one of base sequences SEQ ID NO:125 or 126 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:125 or 126 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:125 or 126 and has a length of 21 nucleotides.

A preferred oligonucleotide comprising or consisting of 2′-O-methyl phosphorothioate RNA comprises one of base sequences SEQ ID NO:127 or 128 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:127 or 128 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:127 or 128 and has a length of 24 nucleotides.

A preferred oligonucleotide comprising or consisting of 2′-O-methyl phosphorothioate RNA comprises one of base sequences SEQ ID NO:129 or 130 and has a length from 27-36 nucleotides, more preferably 27, 28, 29, 30, 31, 32, 33, 34, 35 or 36 nucleotides.

An even more preferred oligonucleotide consists of 2′-O-methyl phosphorothioate RNA and comprises one of the base sequences SEQ ID NO:129 or 130 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:129 or 130 and has a length of 27 nucleotides.

A preferred oligonucleotide comprising or consisting of 2′-O-methyl phosphorothioate RNA comprises one of base sequences SEQ ID NO:131 or 132 and has a length from 30-36 nucleotides, more preferably 30, 31, 32, 33, 34, 35 or 36 nucleotides. An even more preferred oligonucleotide consists of 2′-O-methyl phosphorothioate RNA and comprises one of the base sequences SEQ ID NO:131 or 132 and has a length from 30-36 nucleotides, more preferably 30, 31, 32, 33, 34, 35 or 36 nucleotides. Most preferred oligonucleotide consists of 2′-O-methyl phosphorothioate RNA, has a base sequence that consists of one of the base sequences SEQ ID NO:131 or 132 and has a length of 30 nucleotides.

In another embodiment, an oligonucleotide comprising or consisting of 2′-O-methyl phosphorothioate RNA and comprising a 5-methylcytosine, is represented by a nucleotide sequence comprising or consisting of a sequence that binds (or is able to bind), targets, hybridizes (or is able to hybridize) and/or is reverse complementary to a (CGG)ₙ repeat in a transcript and is particularly useful for the treatment, delay, amelioration and/or prevention of human fragile X syndromes, caused by repeat expansion in the FMR1 gene. Preferably, these genes are from human origin.

In a preferred embodiment, an oligonucleotide defined as being reverse complementary to, binding (or is able to bind), hybridizing (or is able to hybridize) or targeting a (CGG)ₙ repeat comprises or consists of a repetitive nucleotide unit (XXG)ₘ and has a length comprised from 12 to 36 nucleotides and wherein each X is C or 5-methylcytosine, such that at least one X is 5-methylcytosine.

m is an integer. In the context of this embodiment, m may be 4, 5, 6, 7, 8, 9, 10, 11, 12. A preferred value for m is 7.

A more preferred oligonucleotide therefore comprises or consists of a repetitive nucleotide unit (XXG)ₘ, wherein each X is C or 5-methylcytosine, such that at least one X is 5-methylcytosine, and m is an integer from 4 to 12 (SEQ ID NO:22 to 30).

An even more preferred oligonucleotide comprises or consists of a repetitive nucleotide unit (XXG)ₘ, wherein each X is 5-methylcytosine, and m is an integer from 4 to 12 (SEQ ID NO:22 to 30).

An even more preferred oligonucleotide therefore comprises or consists of a repetitive nucleotide unit (XXG)₇ (SEQ ID NO:25), wherein each X is C or 5-methylcytosine, such that at least one X is 5-methylcytosine.

An even more preferred oligonucleotide comprises or consists of a repetitive nucleotide unit (XXG)₇ (SEQ ID NO:25), wherein each X is 5-methylcytosine.

Most preferred oligonucleotides sequences comprising or consisting of a repetitive nucleotide unit (XXG)ₘ have been identified in table 2 as SEQ ID NO:133-146.

A preferred oligonucleotide comprising or consisting of 2′-O-methyl phosphorothioate RNA comprises one of base sequences SEQ ID NO:133 or 134 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:133 or 134 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:133 or 134 and has a length of 12 nucleotides.

A preferred oligonucleotide comprising or consisting of 2′-O-methyl phosphorothioate RNA comprises one of base sequences SEQ ID NO:135 or 136 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:135 or 136 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:135 or 136 and has a length of 15 nucleotides.

A preferred oligonucleotide comprising or consisting of 2′-O-methyl phosphorothioate RNA comprises one of base sequences SEQ ID NO:137 or 138 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:137 or 138 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:137 or 138 and has a length of 18 nucleotides.

A preferred oligonucleotide comprising or consisting of 2′-O-methyl phosphorothioate RNA comprises one of base sequences SEQ ID NO:139 or 140 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:139 or 140 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:139 or 140 and has a length of 21 nucleotides.

A preferred oligonucleotide comprising or consisting of 2′-O-methyl phosphorothioate RNA comprises one of base sequences SEQ ID NO:141 or 142 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:141 or 142 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:141 or 142 and has a length of 24 nucleotides.

A preferred oligonucleotide comprising or consisting of 2′-O-methyl phosphorothioate RNA comprises one of base sequences SEQ ID NO:143 or 144 and has a length from 27-36 nucleotides, more preferably 27, 28, 29, 30, 31, 32, 33, 34, 35 or 36 nucleotides.

An even more preferred oligonucleotide consists of 2′-O-methyl phosphorothioate RNA and comprises one of the base sequences SEQ ID NO:143 or 144 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:143 or 144 and has a length of 27 nucleotides.

A preferred oligonucleotide comprising or consisting of 2′-O-methyl phosphorothioate RNA comprises one of base sequences SEQ ID NO:145 or 146 and has a length from 30-36 nucleotides, more preferably 30, 31, 32, 33, 34, 35 or 36 nucleotides. An even more preferred oligonucleotide consists of 2′-O-methyl phosphorothioate RNA and comprises one of the base sequences SEQ ID NO:145 or 146 and has a length from 30- 36 nucleotides, more preferably 30, 31, 32, 33, 34, 35 or 36 nucleotides. Most preferred oligonucleotide consists of 2′-O-methyl phosphorothioate RNA, has a base sequence that consists of one of the base sequences SEQ ID NO:145 or 146 and has a length of 30 nucleotides.

In another embodiment, an oligonucleotide comprising or consisting of 2′-O-methyl phosphorothioate RNA and comprising a 5-methylcytosine and/or a 5-methyluracil, is represented by a nucleotide sequence comprising or consisting of a sequence that binds (or is able to bind), targets, hybridizes (or is able to hybridize) and/or is reverse complementary to a (GAA)ₙ repeat in a transcript and is particularly useful for the treatment, delay and/or prevention of the human genetic disorder Friedreich's ataxia.

In a preferred embodiment, an oligonucleotide defined as being reverse complementary to, binding (or being able to bind), hybridizing (or being able to hybridize) or targeting a (GAA)ₙ repeat comprises or consists of a repetitive nucleotide unit (YYX)ₘ and has a length comprised from 12 to 36 nucleotides and wherein each X is C or 5-methylcytosine, and each Y is U or 5-methyluracil such that at least one X is 5-methylcytosine and/or at least one Y is 5-methyluracil.

m is an integer. In the context of this embodiment, m may be 4, 5, 6, 7, 8, 9, 10, 11, 12. A preferred value for m is 7.

A more preferred oligonucleotide therefore comprises or consists of a repetitive nucleotide unit (YYX)ₘ, wherein each X is C or 5-methylcytosine, and each Y is U or 5-methyluracil such that at least one X is 5-methylcytosine and/or at least one Y is 5-methyluracil, and m is an integer from 4 to 12 (SEQ ID NO:31 to 39).

An even more preferred oligonucleotide comprises or consists of a repetitive nucleotide unit (YYX)ₘ, wherein each X is 5-methylcytosine, and/or each Y is 5-methyluracil, and m is an integer from 4 to 12 (SEQ ID NO:31 to 39).

An even more preferred oligonucleotide therefore comprises or consists of a repetitive nucleotide unit (YYX)₇ (SEQ ID NO:34), wherein each X is C or 5-methylcytosine, and each Y is U or 5-methyluracil such that at least one X is 5-methylcytosine and/or at least one Y is 5-methyluracil.

An even more preferred oligonucleotide comprises or consists of a repetitive nucleotide unit (YYX)₇ (SEQ ID NO:34), wherein each X is 5-methylcytosine, and/or each Y is 5-methyluracil.

Most preferred oligonucleotides sequences comprising or consisting of a repetitive nucleotide unit (XYG)ₘ have been identified in table 2 as SEQ ID NO:147-167.

A preferred oligonucleotide comprising or consisting of 2′-O-methyl phosphorothioate RNA comprises one of base sequences SEQ ID NO:147 or 148 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:147 or 148 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:147 or 148 and has a length of 12 nucleotides.

A preferred oligonucleotide comprising or consisting of 2′-O-methyl phosphorothioate RNA comprises one of base sequences SEQ ID NO:149 or 150 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:149 or 150 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:149 or 150 and has a length of 15 nucleotides.

A preferred oligonucleotide comprising or consisting of 2′-O-methyl phosphorothioate RNA comprises one of base sequences SEQ ID NO:151 or 152 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:151 or 152 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:151 or 152 and has a length of 18 nucleotides.

A preferred oligonucleotide comprising or consisting of 2′-O-methyl phosphorothioate RNA comprises one of base sequences SEQ ID NO:153-157 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:153-157 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:153-157 and has a length of 21 nucleotides.

A preferred oligonucleotide comprising or consisting of 2′-O-methyl phosphorothioate RNA comprises one of base sequences SEQ ID NO:158 or 159 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:158 or 159 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:158 or 159 and has a length of 24 nucleotides.

A preferred oligonucleotide comprising or consisting of 2′-O-methyl phosphorothioate RNA comprises one of base sequences SEQ ID NO:160 or 161 and has a length from 27-36 nucleotides, more preferably 27, 28, 29, 30, 31, 32, 33, 34, 35 or 36 nucleotides.

An even more preferred oligonucleotide consists of 2′-O-methyl phosphorothioate RNA and comprises one of the base sequences SEQ ID NO:160 or 161 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has a base sequence that consists of one of the base sequences SEQ ID NO:160 or 161 and has a length of 27 nucleotides.

A preferred oligonucleotide comprising or consisting of 2′-O-methyl phosphorothioate RNA comprises one of base sequences SEQ ID NO:162 or 163 and has a length from 30-36 nucleotides, more preferably 30, 31, 32, 33, 34, 35 or 36 nucleotides. An even more preferred oligonucleotide consists of 2′-O-methyl phosphorothioate RNA and comprises one of the base sequences SEQ ID NO:162 or 163 and has a length from 30-36 nucleotides, more preferably 30, 31, 32, 33, 34, 35 or 36 nucleotides. Most preferred oligonucleotide consists of 2′-O-methyl phosphorothioate RNA, has a base sequence that consists of one of the base sequences SEQ ID NO:162 or 163 and has a length of 30 nucleotides.

A preferred oligonucleotide comprising or consisting of 2′-O-methyl phosphorothioate RNA comprises one of base sequences SEQ ID NO:164 or 165 and has a length from 33-36 nucleotides, more preferably 33, 34, 35 or 36 nucleotides. An even more preferred oligonucleotide consists of 2′-O-methyl phosphorothioate RNA and comprises one of the base sequences SEQ ID NO:164 or 165 and has a length from 33-36 nucleotides, more preferably 33, 34, 35 or 36 nucleotides. Most preferred oligonucleotide consists of 2′-O-methyl phosphorothioate RNA, has a base sequence that consists of one of the base sequences SEQ ID NO:164 or 165 and has a length of 33 nucleotides.

A preferred oligonucleotide comprising or consisting of 2′-O-methyl phosphorothioate RNA comprises one of base sequences SEQ ID NO:166 or 167 and has a length of 36 nucleotides. An even more preferred oligonucleotide consists of 2′-O-methyl phosphorothioate RNA and has a base sequence that consists one of the base sequences SEQ ID NO:166 or 167 and has a length of 36 nucleotides.

In another embodiment, an antisense oligonucleotide comprising or consisting of 2′-O-methyl phosphorothioate RNA and comprising a 5-methylcytosine, is represented by a nucleotide sequence comprising or consisting of a sequence that binds to (or is able to bind), hybridizes (or is able to hybridize), targets and/or is reverse complementary to a (CCG)ₙ or (GCC)ₙ repeat in a transcript and is particularly useful for the treatment, delay, amelioration and/or prevention of the human genetic disorder fragile XE mental retardation, caused by repeat expansion in the FMR2 gene. Preferably, these genes are from human origin.

In a preferred embodiment, an oligonucleotide defined as being reverse complementary to, binding (or being able to bind), hybridizing (or being able to hybridize) or targeting a (CCG)ₙ repeat comprises or consists of a repetitive nucleotide unit (XGG)ₘ or (GGX)ₘ and has a length comprised from 12 to 36 nucleotides and wherein each X is C or 5-methylcytosine. m is an integer. In the context of this embodiment, m may be 4, 5, 6, 7, 8, 9, 10, 11, 12. A preferred value for m is 7.

A more preferred oligonucleotide therefore comprises or consists of a repetitive nucleotide unit (XGG)ₘ or (GGX)ₘ, wherein each X is C or 5-methylcytosine, and m is an integer from 4 to 12 (SEQ ID NO:49 to 57) or (SEQ ID NO:40 to 48).

An even more preferred oligonucleotide comprises or consists of a repetitive nucleotide unit (XGG)ₘ or (GGX)ₘ, wherein each X is 5-methylcytosine, and m is an integer from 4 to 12 (SEQ ID NO:49 to 57) or (SEQ ID NO:40 to 48).

An even more preferred oligonucleotide therefore comprises or consists of a repetitive nucleotide unit (XGG)₇ (SEQ ID NO:52) or (GGX)₇ (SEQ ID NO:43), wherein each X is C or 5-methylcytosine.

An even more preferred oligonucleotide comprises or consists of a repetitive nucleotide unit (XGG)₇ (SEQ ID NO:52) or (GGX)₇ (SEQ ID NO:43), wherein each X is 5-methylcytosine.

Most preferred oligonucleotides sequences comprising or consisting of a repetitive nucleotide unit (GGX)ₘ have been identified in table 2 as SEQ ID NO:168-177.

A preferred oligonucleotide comprising or consisting of 2′-O-methyl phosphorothioate RNA comprises base sequence SEQ ID NO:168 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequences SEQ ID NO:168 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequences SEQ ID NO:168 and has a length of 12 nucleotides.

A preferred oligonucleotide comprising or consisting of 2′-O-methyl phosphorothioate RNA comprises base sequence SEQ ID NO:169 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:169 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:169 and has a length of 15 nucleotides.

A preferred oligonucleotide comprising or consisting of 2′-O-methyl phosphorothioate RNA comprises base sequence SEQ ID NO:170 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:170 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:170 and has a length of 18 nucleotides.

A preferred oligonucleotide comprising or consisting of 2′-O-methyl phosphorothioate RNA comprises one of base sequences SEQ ID NO:171-174 has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:171-174 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:171-174 and has a length of 21 nucleotides.

A preferred oligonucleotide comprising or consisting of 2′-O-methyl phosphorothioate RNA comprises base sequence SEQ ID NO:175 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:175 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:175 and has a length of 24 nucleotides.

A preferred oligonucleotide comprising or consisting of 2′-O-methyl phosphorothioate RNA comprises base sequence SEQ ID NO:176 and has a length from 27-36 nucleotides, more preferably 27, 28, 29, 30, 31, 32, 33, 34, 35 or 36 nucleotides. An even more preferred oligonucleotide consists of 2′-O-methyl phosphorothioate RNA and comprises base sequence SEQ ID NO:176 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:176 and has a length of 27 nucleotides.

A preferred oligonucleotide comprising or consisting of 2′-O-methyl phosphorothioate RNA comprises base sequence SEQ ID NO:177 and has a length from 30-36 nucleotides, more preferably 30, 31, 32, 33, 34, 35 or 36 nucleotides. An even more preferred oligonucleotide consists of 2′-O-methyl phosphorothioate RNA and comprises base sequence SEQ ID NO:177 and has a length from 30-36 nucleotides, more preferably 30, 31, 32, 33, 34, 35 or 36 nucleotides. Most preferred oligonucleotide consists of 2′-O-methyl phosphorothioate RNA, has its base sequence that consists of base sequence SEQ ID NO:177 and has a length of 30 nucleotides.

Most preferred oligonucleotides sequences comprising or consisting of a repetitive nucleotide unit (XGG)ₘ have been identified in table 2 as SEQ ID NO:178-184.

A preferred oligonucleotide comprising or consisting of 2′-O-methyl phosphorothioate RNA comprises base sequence SEQ ID NO:178 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:178 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:178 and has a length of 12 nucleotides.

A preferred oligonucleotide comprising or consisting of 2′-O-methyl phosphorothioate RNA comprises base sequence SEQ ID NO:179 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:179 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:179 and has a length of 15 nucleotides.

A preferred oligonucleotide comprising or consisting of 2′-O-methyl phosphorothioate RNA comprises base sequences SEQ ID NO:180 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:180 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:180 and has a length of 18 nucleotides.

A preferred oligonucleotide comprising or consisting of 2′-O-methyl phosphorothioate RNA comprises base sequence SEQ ID NO:181 and has a length from 21-36 nucleotides, more preferably 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:181 and has a length from 21-36 nucleotides, more preferably 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:181 and has a length of 21 nucleotides.

A preferred oligonucleotide comprising or consisting of 2′-O-methyl phosphorothioate RNA comprises base sequence SEQ ID NO:182 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:182 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:182 and has a length of 24 nucleotides.

A preferred oligonucleotide comprising or consisting of 2′-O-methyl phosphorothioate RNA comprises base sequence SEQ ID NO:183 and has a length from 27-36 nucleotides, more preferably 27, 28, 29, 30, 31, 32, 33, 34, 35 or 36 nucleotides. An even more preferred oligonucleotide consists of 2′-O-methyl phosphorothioate RNA and comprises base sequence SEQ ID NO:183 and has a length from 27-36 nucleotides, more preferably 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:183 and has a length of 27 nucleotides.

A preferred oligonucleotide comprising or consisting of 2′-O-methyl phosphorothioate RNA comprises base sequence SEQ ID NO:184 and has a length from 30-36 nucleotides, more preferably 30, 31, 32, 33, 34, 35 or 36 nucleotides. An even more preferred oligonucleotide consists of 2′-O-methyl phosphorothioate RNA and comprises base sequence SEQ ID NO:184 and has a length from 30-36 nucleotides, more preferably 30, 31, 32, 33, 34, 35 or 36 nucleotides. Most preferred oligonucleotide consists of 2′-O-methyl phosphorothioate RNA, has its base sequence that consists of base sequence SEQ ID NO:184 and has a length of 30 nucleotides.

In another embodiment, an oligonucleotide comprising or consisting of 2′-O-methyl phosphorothioate RNA and comprising a 5-methylcytosine and/or a 2,6-diaminopurine, is represented by a nucleotide sequence comprising or consisting of a sequence that binds (or is able to bind), hybridizes (or is able to hybridize), targets and/or is reverse complementary to a (CCUG)ₙ repeat in a transcript and is particularly useful for the treatment, delay and/or prevention of the human genetic disorder myotonic dystrophy type 2 (DM2), caused by repeat expansions in the DM2/ZNF9 gene. Preferably, these genes are from human origin.

In a preferred embodiment, an oligonucleotide defined as being reverse complementary to, binding (or being able to bind), hybridizing (or being able to hybridize) or targeting a (CCUG)ₙ repeat comprises or consists of a repetitive nucleotide unit (XZGG)ₘ and has a length comprised from 12 to 36 nucleotides and wherein each X is C or 5-methylcytosine, and each Z is A or 2,6-diaminopurine such that at least one X is 5-methylcytosine and/or at least one Z is 2,6-diaminopurine.

m is an integer. In the context of this embodiment, m may be 3, 4, 5, 6, 7, 8, 9. A preferred value for m is 5.

A more preferred oligonucleotide therefore comprises or consists of a repetitive nucleotide unit (XZGG)ₘ, wherein each X is C or 5-methylcytosine, and each Z is A or 2,6-diaminopurine such that at least one X is 5methyl-cytosine and/or at least one A is 2,6-diaminopurine, and m is an integer from 3 to 9 (SEQ ID NO:63 to 69).

An even more preferred oligonucleotide comprises or consists of a repetitive nucleotide unit (XZGG)ₘ, wherein each X is 5-methylcytosine, and/or each Z is 2,6-diaminopurine, and m is an integer from 3 to 9 (SEQ ID NO:63 to 69).

An even more preferred oligonucleotide therefore comprises or consists of a repetitive nucleotide unit (XZGG)₅ (SEQ ID NO:65), wherein each X is C or 5-methylcytosine, and each Z is A or 2,6-diaminopurine such that at least one X is 5-methylcytosine and/or at least one Z is 2,6-diaminopurine.

An even more preferred oligonucleotide comprises or consists of a repetitive nucleotide unit (XZGG)₅ (SEQ ID NO:65), wherein each X is 5-methyl-cytosine, and/or each Z is 2,6-diaminopurine.

Most preferred oligonucleotides sequences comprising or consisting of a repetitive nucleotide unit (XZGG)ₘ have been identified in table 2 as SEQ ID NO:193-208.

A preferred oligonucleotide comprising or consisting of 2′-O-methyl phosphorothioate RNA comprises one of base sequences SEQ ID NO:193 or 194 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:193 or 194 and has a length from 12-36 nucleotides, more preferably 12, 13, 14, 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:193 or 194 and has a length of 12 nucleotides.

A preferred oligonucleotide comprising or consisting of 2′-O-methyl phosphorothioate RNA comprises one of base sequences SEQ ID NO:195 or 196 and has a length from 16-36 nucleotides, more preferably 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:195 or 196 and has a length from 16-36 nucleotides, more preferably 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:195 or 196 and has a length of 16 nucleotides.

A preferred oligonucleotide comprising or consisting of 2′-O-methyl phosphorothioate RNA comprises one of base sequences SEQ ID NO:197-200 and has a length from 20-36 nucleotides, more preferably 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO: 197-200 and has a length from 20-36 nucleotides, more preferably 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:197-200 and has a length of 20 nucleotides.

A preferred oligonucleotide comprising or consisting of 2′-O-methyl phosphorothioate RNA comprises one of base sequences SEQ ID NO:201 or 202 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:201 or 202 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:201 or 202 and has a length of 24 nucleotides.

A preferred oligonucleotide comprising or consisting of 2′-O-methyl phosphorothioate RNA comprises one of base sequences SEQ ID NO:203 or 204 and has a length from 28-36 nucleotides, more preferably 28, 29, 30, 31, 32, 33, 34, 35 or 36 nucleotides. An even more preferred oligonucleotide consists of 2′-O-methyl phosphorothioate RNA and comprises one of the base sequences SEQ ID NO:203 or 204 and has a length from 28-36 nucleotides, more preferably 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:203 or 204 and has a length of 28 nucleotides.

A preferred oligonucleotide comprising or consisting of 2′-O-methyl phosphorothioate RNA comprises one of base sequences SEQ ID NO:205 or 206 and has a length from 32-36 nucleotides, more preferably 32, 33, 34, 35 or 36 nucleotides. An even more preferred oligonucleotide consists of 2′-O-methyl phosphorothioate RNA and comprises one of the base sequences SEQ ID NO:205 or 206 and has a length from 32-36 nucleotides, more preferably 32, 33, 34, 35 or 36 nucleotides. Most preferred oligonucleotide consists of 2′-O-methyl phosphorothioate RNA, has its base sequence that consists of one of the base sequences SEQ ID NO:205 or 206 and has a length of 32 nucleotides.

A preferred oligonucleotide comprising or consisting of 2′-O-methyl phosphorothioate RNA comprises one of base sequences SEQ ID NO:207 or 208 and has a length of 36 nucleotides. An even more preferred oligonucleotide consists of 2′-O-methyl phosphorothioate RNA and has its base sequence that consists of one of the base sequences SEQ ID NO:207 or 208 and has a length of 36 nucleotides.

In another embodiment, an oligonucleotide comprising or consisting of 2′-O-methyl phosphorothioate RNA and comprising a 5-methyluracil and/or a 2,6-diaminopurine, is represented by a nucleotide sequence comprising or consisting of a sequence that binds (or is able to bind), hybridizes (or is able to hybridize), targets and/or is reverse complementary to a (AUUCU)ₙ repeat in an intron and is particularly useful for the treatment, delay, amelioration and/or prevention of the human genetic disorder spinocerebellar ataxia type 10 (SCA10). Preferably, this gene is from human origin. In a preferred embodiment, an oligonucleotide defined as being reverse complementary to, binding (or being able to bind), hybridizing (or being able to hybridize) or targeting a (AUUCU)ₙ repeat comprises or consists of a repetitive nucleotide unit (ZGZZY)ₘ and has a length comprised from 12 to 36 nucleotides and wherein each Y is U or 5-methyluracil, and each Z is A or 2,6-diaminopurine such that at least one Y is 5-methyluracil and/or at least one Z is 2,6-diaminopurine.

m is an integer. In the context of this embodiment, m may be 3, 4, 5, 6, 7. A preferred value for m is 4.

A more preferred oligonucleotide therefore comprises or consists of a repetitive nucleotide unit (ZGZZY)ₘ, wherein each Y is U or 5-methyluracil, and each Z is A or 2,6-diaminopurine such that at least one Y is 5-methyluracil and/or at least one Z is 2,6-diaminopurine, and m is an integer from 3 to 7 (SEQ ID NO:58 to 62).

An even more preferred oligonucleotide comprises or consists of a repetitive nucleotide unit (ZGZZY)ₘ, wherein each Y is 5-methyluracil, and/or each Z is 2,6-diaminopurine, and m is an integer from 3 to 7 (SEQ ID NO:58 to 62).

An even more preferred oligonucleotide therefore comprises or consists of a repetitive nucleotide unit (ZGZZY)₄ (SEQ ID NO:59), wherein each Y is C or 5-methyluracil, and each Z is A or 2,6-diaminopurine such that at least one Y is 5-methyluracil and/or at least one Z is 2,6-diaminopurine.

An even more preferred oligonucleotide comprises or consists of a repetitive nucleotide unit (ZGZZY)₄ (SEQ ID NO:59), wherein each Y is 5-methyluracil, and/or each Z is 2,6-diaminopurine.

Most preferred oligonucleotides sequences comprising or consisting of a repetitive nucleotide unit (ZGZZY)ₘ have been identified in table 2 as SEQ ID NO:185-192.

A preferred oligonucleotide comprising or consisting of 2′-O-methyl phosphorothioate RNA comprises base sequence SEQ ID NO:185 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:185 and has a length from 15-36 nucleotides, more preferably 15, 16, 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:185 and has a length of 15 nucleotides.

A preferred oligonucleotide comprising or consisting of 2′-O-methyl phosphorothioate RNA comprises one of base sequences SEQ ID NO:186-189 and has a length from 20-36 nucleotides, more preferably 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO: 186-189 and has a length from 20-36 nucleotides, more preferably 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:186-189 and has a length of 20 nucleotides.

A preferred oligonucleotide comprising or consisting of 2′-O-methyl phosphorothioate RNA comprises base sequence SEQ ID NO:190 and has a length from 25-36 nucleotides, more preferably 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:190 and has a length from 25-36 nucleotides, more preferably 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:190 and has a length of 25 nucleotides.

A preferred oligonucleotide comprising or consisting of 2′-O-methyl phosphorothioate RNA comprises base sequence SEQ ID NO:191 and has a length from 30-36 nucleotides, more preferably 30, 31, 32, 33, 34, 35 or 36 nucleotides. An even more preferred oligonucleotide consists of 2′-O-methyl phosphorothioate RNA and comprises base sequence SEQ ID NO:191 and has a length from 30-36 nucleotides, more preferably 30, 31, 32, 33, 34, 35 or 36 nucleotides. Most preferred oligonucleotide consists of 2′-O-methyl phosphorothioate RNA, has its base sequence that consists of base sequence SEQ ID NO:191 and has a length of 30 nucleotides.

A preferred oligonucleotide comprising or consisting of 2′-O-methyl phosphorothioate RNA comprises base sequence SEQ ID NO:192 and has a length from 35-36 nucleotides, more preferably 35 or 36 nucleotides. An even more preferred oligonucleotide consists of 2′-O-methyl phosphorothioate RNA and comprises base sequence SEQ ID NO:192 and has a length from 35-36 nucleotides, more preferably 35 or 36 nucleotides. Most preferred oligonucleotide consists of 2′-O-methyl phosphorothioate RNA, has its base sequence that consists of base sequence SEQ ID NO:192 and has a length of 35 nucleotides.

In another embodiment, an oligonucleotide comprising or consisting of 2′-O-methyl phosphorothioate RNA and comprising a 5-methylcytosine and/or a abasic monomer, and/or a inosine, is represented by a nucleotide sequence comprising or consisting of a sequence that binds (or is able to bind), hybridizes (or is able to hybridize), targets and/or is reverse complementary to a (GGGGCC)ₙ repeat present in a C9ORF72 human transcript and is particularly useful for the treatment, delay, amelioration and/or prevention of the human genetic disorder amylotrophic lateral sclerosis (ALS) or frontotemporal dementia (FTD). Preferably, this gene is from human origin.

In a preferred embodiment, an oligonucleotide defined as being reverse complementary to, binding (or being able to bind), hybridizing (or being able to hybridize) or targeting a (GGGGCC)ₙ repeat comprises or consists of a repetitive nucleotide unit (GGXUXX)ₘ, (GGXQXX)ₘ, (GGXIXX)ₘ, or (GGCCUC)ₘ, and has a length comprised from 17 to 36 nucleotides and wherein each X is C or 5-methylcytosine such that at least one X is 5-methylcytosine, wherein each Q is an abasic monomer, wherein each I is an inosine, and wherein m is an integer. In the context of this embodiment, m may be 3, 4, 5, 6, 7. A preferred value for m is 3 or 4.

More preferably, said oligonucleotide comprises or consists of a repetitive nucleotide unit SEQ ID NO:216-219 as defined in table 1. Even more preferred oligonucleotides sequences comprising or consisting of a repetitive nucleotide unit (GGXUXX)ₘ, (GGXQXX)ₘ, (GGXIXX)ₘ, or (GGCCUC)ₘ, have been identified in table 2 as SEQ ID NO:209-215.

A preferred oligonucleotide comprising or consisting of 2′-O-methyl phosphorothioate RNA comprises one of base sequences SEQ ID NO:209 or 211 and has a length from 17-36 nucleotides, more preferably 17, 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:209 or 211 and has a length from 17-36 nucleotides, more preferably 17, 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:209 or 211 and has a length of 17 or 18 nucleotides.

A preferred oligonucleotide comprising or consisting of 2′-O-methyl phosphorothioate RNA comprises base sequence SEQ ID NO:210 and has a length from 18-36 nucleotides, more preferably 18, 19, 20, 21, 22, 23, 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:210 and has a length from 18-36 nucleotides, more preferably 18, 19, 20, 21, 22, 23, 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequence SEQ ID NO:210 and has a length of 18 nucleotides.

A preferred oligonucleotide comprising or consisting of 2′-O-methyl phosphorothioate RNA comprises one of base sequences SEQ ID NO:212 or 215 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises one of the base sequences SEQ ID NO:212 or 215 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of one of the base sequences SEQ ID NO:212 or 215 and has a length of 24 nucleotides.

A preferred oligonucleotide comprising or consisting of 2′-O-methyl phosphorothioate RNA comprises base sequence SEQ ID NO:213 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:213 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequences SEQ ID NO:213 and has a length of 24 nucleotides.

A preferred oligonucleotide comprising or consisting of 2′-O-methyl phosphorothioate RNA comprises base sequence SEQ ID NO:214 and has a length from 24-36 nucleotides, more preferably 24, 25, 26, 27, 28, 29, 30, 31, 32, 33, 34, 35 or 36 nucleotides. An even more preferred oligonucleotide consists of 2′-O-methyl phosphorothioate RNA and comprises base sequence SEQ ID NO:214 and has a length from 24-36 nucleotides, more preferably 24, 25, 26, 27, 28, 29, 30, 31, 32, 33, 34, 35 or 36 nucleotides. Most preferred oligonucleotide consists of 2′-O-methyl phosphorothioate RNA, has its base sequence that consists of base sequences SEQ ID NO:214 and has a length of 24 nucleotides.

In an embodiment, an oligonucleotide preferably comprises or consists of 2′-O-methyl phosphorothioate RNA, comprises a 5-methylcytosine and/or a 5-methyluracil and/or a 2,6-diaminopurine base, is represented by a nucleotide sequence comprising or consisting of at least 12 to 36 consecutive nucleotides, said oligonucleotide targeting, hybridizing (or is able to hybridize), binding (or is able to bind) and/or being reverse complementary to a repeat as earlier defined herein More preferably, said nucleotide sequence comprising or consisting of at least 12 to 36 nucleotides, even more preferably 15 to 24, and most preferably 20 or 21 nucleotides. The length of said oligonucleotide may be 12, 13, 14, 15, 16, 17, 18, 19, 20, 21, 22, 23, 24, 25, 26, 27, 28, 29, 30, 31, 32, 33, 34, 35, or 36 nucleotides. Said oligonucleotide may be reverse complementary to and/or capable of hybridizing to and/or capable of targeting and/or capable of binding to a repeat in a coding region of a transcript, preferably a polyglutamine (CAG)ₙ coding tract. Said oligonucleotide may also be reverse complementary to and/or capable of hybridizing to and/or capable of targeting and/or capable of binding to a non-coding region for instance 5′ or 3′ untranslated regions, or intronic sequences present in precursor RNA molecules.

In the context of the invention, the expression “capable of” may be replaced with “is able to”.

In a second aspect, the present invention relates to an oligonucleotide, which comprises one or more abasic sites, as defined further below, at one or both termini. Preferably 1 to 10, more preferably 2, 3, 4, 5, 6, 7, 8, 9 or 10 and most preferably 4 abasic sites are present at a single terminus or at both termini of the oligonucleotide. One or more abasic sites may be present and both free termini of the oligonucleotide (5′ and 3′), or at only one. The oligonucleotide according to this aspect of the invention preferably is represented by a nucleotide or a base sequence comprising or consisting of a sequence that binds (or is able to bind), hybridizes (or is able to hybridize), targets and/or is reverse complementary to a repetitive element in a RNA transcript selected from the (CAG)ₙ, (GCG)ₙ, (CGG)ₙ, (GAA)ₙ, (GCC)ₙ, (CCG)ₙ, (AUUCU)ₙ, (GGGGCC)ₙ or (CCUG)ₙ, as indicated above. Said oligonucleotide is preferably a single stranded oligonucleotide, and may further optionally comprise any of the modifications as discussed herein, such as one or more base modifications, sugar modifications and/or backbone modifications, such as 5-methyl-C, 5-methyl-U, 2,6-diaminopurine, 2′-O-methyl, phosphorothioate, and combinations thereof. It is to be understood that in this aspect of the invention, these modification are not compulsory.

The oligonucleotide according to this aspect of the invention, comprising one or more abasic sites at one or both termini has an improved parameter over the oligonucleotides without such abasic sites. In this context, parameters may include: binding affinity and/or kinetics, silencing activity, allelic selectivity, biostability, (intra-tissue) distribution, cellular uptake and/or trafficking, and/or immunogenicity of said oligonucleotide, as explained earlier herein in connection with the improved parameter of an oligonucleotide of the invention of the first aspect. Each of the assays and definitions provided herein in connection with the improvement of a parameter of an oligonucleotide of the first aspect also hold for an oligonucleotide of the second aspect.

Below; an oligonucleotide comprising or consisting of 2′-O-methyl phosphorothioate RNA, comprising a 5-methylcytosine and/or a 5-methyluracil base and being represented by a nucleotide or a base sequence comprising (CUG)ₘ and thus binding to (or being able to bind to), hybridizing (or being able to hybridize), targeting and/or being reverse complementary to (CAG)ₙ is taken as an example to further illustrate the invention. Similar parameters defined in the context of such oligonucleotide could be defined by the skilled person for other oligonucleotides falling under the scope of the invention and binding to (or being able to bind to), hybridizing (or being able to hydridize), targeting and/or being reverse complementary to other repeats as identified herein. Other or similar symptoms may be identified by the skilled person concerning other diseases as identified herein.

In a preferred embodiment, in the context of the invention, an oligonucleotide as designed herein is able to delay and/or cure and/or treat and/or prevent and/or ameliorate a human genetic disorder as Huntington's disease (HD), spinocerebellar ataxia (SCA) type 1, 2, 3, 6, 7, 12 or 17, amyotrophic lateral sclerosis (ALS), frontotemporal dementia (FTD), X-linked spinal and bulbar muscular atrophy (SBMA) and/or dentatorubropallidoluysian atrophy (DRPLA) caused by CAG repeat expansions in the transcripts of a HTT (SEQ ID NO:80), ATXN1 (SEQ ID NO:81), ATXN2 (SEQ ID NO:82) ATXN3 (SEQ ID NO:83), CACNA1A (SEQ ID NO:84), ATXN7 (SEQ ID NO:85), PPP2R2B (SEQ ID NO:86), TBP (SEQ ID NO:87), AR (SEQ ID NO:88), ATN1 (SEQ ID NO:89) genes when this oligonucleotide is able to reduce or decrease the amount of (toxic) transcript of a diseased allele of a HTT, ATXN1, ATXN2 ATXN3, CACNA1A, ATXN7, PPP2R2B, TBP, AR or ATN1 gene in a cell of a patient, in a tissue of a patient and/or in a patient. In an embodiment, said HTT, ATXN1, ATXN2 ATXN3, CACNA1A, ATXN7, PPP2R2B, TBP, AR or ATN1 genes are human genes.

In the case of HD, an expanded CAG repeat region is present in exon 1 of the HTT gene in the genome of a patient. An expanded CAG repeat region may be defined herein as comprising a consecutive repetition of 38 to 180 repetitive units comprising a CAG trinucleotide, in a transcribed sequence of the HTT gene

In the case of SCA1, an expanded CAG repeat region is present in exon 8 of the ATXN1 gene in the genome of a patient. An expanded CAG repeat region may be defined herein as comprising a consecutive repetition of 41 to 83 repetitive units comprising a CAG trinucleotide, in a transcribed sequence of the ATXN1 gene.

In the case of SCA2, an expanded CAG repeat region is present in exon 1 of the ATXN2 gene in the genome of a patient. An expanded CAG repeat region may be defined herein as comprising a consecutive repetition of 32 to 200 repetitive units comprising a CAG trinucleotide in a transcribed sequence of the ATXN2 gene.

In the case of SCA3, an expanded CAG repeat region is present in exon 8 of the ATXN3 gene in the genome of a patient. An expanded CAG repeat region may be defined herein as comprising a consecutive repetition of 52 to 86 repetitive units comprising a CAG trinucleotide in a transcribed sequence of the ATXN3 gene.

In the case of SCA6, an expanded CAG repeat region is present in exon 47 of the CACNA1A gene in the genome of a patient. An expanded CAG repeat region may be defined herein as comprising a consecutive repetition of 20 to 33 repetitive units comprising a CAG trinucleotide in a transcribed sequence of the CACNA1A gene.

In the case of SCA7, an expanded CAG repeat region is present in exon 3 of the ATXN7 gene in the genome of a patient. An expanded CAG repeat region may be defined herein as comprising a consecutive repetition of 36 to at least 460 repetitive units comprising a CAG trinucleotide in a transcribed sequence of the ATXN7 gene.

In the case of SCA12, an expanded CAG repeat region may be present in the 5′ untranslated region (UTR), in an intron or within an open reading frame of the PPP2R2B gene in the genome of a patient. An expanded CAG repeat region may be defined herein as comprising a consecutive repetition of 66 to 78 repetitive units comprising a CAG trinucleotide in a transcribed sequence of the PPP2R2B gene.

In the case of SCA17, an expanded CAG repeat region is present in exon 3 of the TBP gene in the genome of a patient. An expanded CAG repeat region may be defined herein as comprising a consecutive repetition of 45 to 66 repetitive units comprising a CAG trinucleotide in a transcribed sequence of the TBP gene.

In the case of ALS or FTD, an expanded CAG repeat region is present in exon 1 of the ATXN2 gene in the genome of a patient. An expanded CAG repeat region may be defined herein as comprising a consecutive repetition of 27 to 33 repetitive units comprising a CAG trinucleotide in a transcribed sequence of the ATXN2 gene.

In the case of ALS or FTD, an expanded GGGGCC repeat region is present in the first intron of the C9ORF72 gene in the genome of a patient. An expanded GGGGCC repeat region may be defined herein as comprising a consecutive repetition of >30 repetitive units comprising a GGGGCC hexanucleotide in a transcribed sequence of the C9ORF72 gene.

In the case of SBMA, an expanded CAG repeat region is present in exon 1 of the AR gene in the genome of a patient. An expanded CAG repeat region may be defined herein as comprising a consecutive repetition of 40 repetitive units comprising a CAG trinucleotide in a transcribed sequence of the AR gene.

In the case of DRPLA, an expanded CAG repeat region is present in exon 5 of the ATN1 gene in the genome of a patient. An expanded CAG repeat region may be defined herein as comprising a consecutive repetition of 49 to 88 repetitive units comprising a CAG trinucleotide in a transcribed sequence of the ATN1 gene.

Throughout the invention, the term CAG repeat may be replaced by (CAG)ₙ, and vice versa, wherein n is an integer that may be 6 to 29 when the repeat is present in exon 1 of the HTT transcript of a healthy individual, 6 to 39 when the repeat is present in exon 8 of the ATXN1 gene of a healthy individual, less than 31 when the repeat is present in exon 1 of the ATXN2 gene of a healthy individual, 12 to 40 when the repeat is present in exon 8 of the ATXN3 gene of a healthy individual, less than 18 when the repeat is present in exon 47 of the CACNA1A gene of a healthy individual, 4 to 17 when the repeat is present in exon 3 of the ATXN7 gene of a healthy individual, 7 to 28 when the repeat is present in the 5′UTR of the PPP2R2B gene of a healthy individual, 25 to 42 when the repeat is present in exon 3 of the TBP gene of a healthy individual, 13 to 31 when the repeat is present in exon 1 of the AR gene of a healthy individual, 12 to 40 when the repeat is present in exon 8 of the ATXN3 gene of a healthy individual, or 6 to 35 when the repeat is present in exon 5 of the ATN1 gene of a healthy individual.

It preferably means that an oligonucleotide of the invention reduces a detectable amount of disease-associated or disease-causing or mutant transcript containing an extending or unstable number of CAG repeats in a cell of said patient, in a tissue of said patient and/or in a patient. Alternatively or in combination with previous sentence, said oligonucleotide may reduce the translation of said mutant transcript and thus the amount of mutant (toxic) protein. The reduction or decrease of the amount of expanded CAG repeat transcripts may be at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the amount of expanded CAG repeat transcripts before the treatment. Another parameter may be the decrease in (CAG)ₙ transcript or of the quantity of said mutant transcript. This may be of at least. 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity of said transcript detected at the onset of the treatment

The reduction or decrease may be assessed by Northern Blotting or Q-RT-PCR, preferably as carried out in the experimental part. An oligonucleotide of the invention may first be tested in the cellular system as described in Example 1 in the experimental part.

Alternatively or in combination with previous preferred embodiment, in the context of the invention, an oligonucleotide as designed herein is able to delay and/or cure and/or treat and/or prevent and/or ameliorate a human genetic disorder as Huntington's disease (HD), spinocerebellar ataxia (SCA) type 1, 2, 3, 6, 7, 12 or 17, amyotrophic lateral sclerosis (ALS), frontotemporal dementia (FTD), X-linked spinal and bulbar muscular atrophy (SBMA) and/or dentatorubropallidoluysian atrophy (DRPLA) caused by CAG repeat expansions in the transcripts of the HTT, ATXN1, ATXN2 ATXN3, CACNA1A, ATXN7, PPP2R2B, TBP, AR or ATN1 genes when this oligonucleotide is able to alleviate one or more symptom(s) and/or characteristic(s) and/or to improve a parameter linked with or associated with Huntington's disease (HD), spinocerebellar ataxia (SCA) type 1, 2, 3, 6, 7, 12 or 17, amyotrophic lateral sclerosis (ALS), frontotemporal dementia (FTD), X-linked spinal and bulbar muscular atrophy (SBMA) and/or dentatorubropallidoluysian atrophy (DRPLA) in an individual. An oligonucleotide as defined herein is able to improve one parameter or reduce a symptom or characteristic if after at least one week, one month, six month, one year or more of treatment using a dose of said oligonucleotide of the invention as identified herein said parameter is said to have been improved or said symptom or characteristic is said to have been reduced.

Improvement in this context may mean that said parameter had been significantly changed towards a value of said parameter for a healthy person and/or towards a value of said parameter that corresponds to the value of said parameter in the same individual at the onset of the treatment.

Reduction or alleviation in this context may mean that said symptom or characteristic had been significantly changed towards the absence of said symptom or characteristic which is characteristic for a healthy person and/or towards a change of said symptom or characteristic that corresponds to the state of the same individual at the onset of the treatment.

In this context, symptoms for Huntington's Disease are choreiform movements, progressive dementia and psychiatric manifestations (depression, psychosis, etc.). Choreiform movements consist of involuntary, rapid, irregular, jerky motor actions including facial twitching or writhing and twitching of distal extremities, and later more generalized forms that may impair gait (Ropper and Brown, 2005). Each of these symptoms may be assessed by the physician using known and described methods. A preferred method is monitoring of total functional capacity (TFC), a validated scale or symptom progression regarding the three main symptomatic areas of HD, measured by validated rating scales. These areas are specifically progression of motor signs, progression of neuropsychiatric symptoms and progression of cognitive decline. Another preferred scale therefore is the Unified HD Rating Scale (UHDRS; Huntington Study Group (Kieburtz K. et al. 1996;11:136-142).

Huntington's disease (HD), spinocerebellar ataxia (SCA) type 1, 2, 3, 6, 7, or 17, X-linked spinal and bulbar muscular atrophy (SBMA) and dentatorubropallidoluysian atrophy (DRPLA) are all caused by CAG triplet repeat expansions in the coding region of the gene. Although the disease causing proteins in these diseases are different, in each case the resulting expanded stretch of glutamines results in a toxic-gain-of function of the protein and this leads to neurodegeneration. Protein aggregates are found in the nucleus and cytoplasm of cells, indicating that protein misfolding is a common feature of these disorders. A common preferred parameter is therefore (mutant) protein levels which can be determined by western blot analysis (Evers et al.,), or the presence of protein aggregates in the nucleus and/or cytoplasm which can be monitored by in situ hybridization. An improvement of a HD parameter may be the decrease in the detection of the quantity or amount of protein aggregate. Such decrease may be at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity or amount of protein aggregate before the onset of the treatment.

In the context of HD, various other proteins have been found to co-localize with htt aggregates, i.e. TATA box binding protein (TBP), CREB binding protein (CBP) and several molecular chaperones (Huang et al.,; Muchowski et al.,; Roon-Mom et al.,; Steffan et al.,). Also many affected cellular processes have been identified in HD, such as transcriptional de-regulation, mitochondrial dysfunction, and impaired vesicle transport, which may provide alternative parameters for HD (Bauer et al., 2009; Ross et al.,). An improvement of each of these possible alternative HD parameters (i.e. TATA box binding protein (TBP), CREB binding protein (CBP) and several molecular chaperones) may be defined as for the improvement of protein aggregate as defined above.

### Composition

In a second aspect, there is provided a composition comprising an oligonucleotide as described in the previous section entitled “Oligonucleotide”. This composition preferably comprises or consists of or essentially consists of an oligonucleotide as described above.

As explained in the first aspect of the invention for ALS and FTD, it is known that at least two distinct repeats in at least two distinct transcripts may be involved in, responsible for, or linked with the disease. All preferred features relating to each of these oligonucleotides have been disclosed in the section entitled “oligonucleotide”.

In a preferred embodiment, said composition is for use as a medicament. Said composition is therefore a pharmaceutical composition. A pharmaceutical composition usually comprises a pharmaceutically accepted carrier, diluent and/or excipient. In a preferred embodiment, a composition of the current invention comprises a compound as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000. The compound as described in the invention possesses at least one ionizable group. An ionizable group may be a base or acid, and may be charged or neutral. An ionizable group may be present as ion pair with an appropriate counterion that carries opposite charge(s). Examples of cationic counterions are sodium, potassium, cesium, Tris, lithium, calcium, magnesium, trialkylammonium, triethylammonium, and tetraalkylammonium. Examples of anionic counterions are chloride, bromide, iodide, lactate, mesylate, acetate, trifluoroacetate, dichloroacetate, and citrate. Examples of counterions have been described [e.g. Kumar L. et al, 2008, which is incorporated here in its entirety by reference].

A pharmaceutical composition may be further formulated to further aid in enhancing the stability, solubility, absorption, bioavailability, pharmacokinetics and cellular uptake of said compound, in particular formulations comprising excipients capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, gold nanoparticles, magnetic nanoparticles, silica nanoparticles, lipid nanoparticles, sugar particles, protein nanoparticles and peptide nanoparticles.

A preferred composition comprises at least one excipient that may further aid in enhancing the targeting and/or delivery of said composition and/or said oligonucleotide to and/or into muscle and/or brain tissue and/or to a neuronal tissue and/or a cell. A cell may be a muscular or a neuronal cell.

Many of these excipients are known in the art (e.g. see Bruno, 2011) and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine)), lipids {e.g. 1,2-dioleoyl-3-dimethylammonium propane (DODAP), dioleoyldimethylammonium chloride (DODAC), phosphatidylcholine derivatives [e.g. 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyeline, 2-{3-[bis-(3-amino-propyl)-amino]-propylamino}-N-ditetracedyl carbamoyl methylacetamide (RPR209120), phosphoglycerol derivatives [e.g. 1,2-dipalmitoyl-sn-glycero-3-phosphoglycerol sodium salt (DPPG-Na), phosphaticid acid derivatives [1,2-distearoyl-sn-glycero-3-phosphaticid acid, sodium salt (DSPA), phosphatidylethanolamine derivatives [e.g. dioleoyl-phosphatidylethanolamine (DOPE), 1,2-distearoyl-sn-glycero-3-phosphoethanolamine (DSPE), 2-diphytanoyl-sn-glycero-3-phosphoethanolamine (DPhyPE),], N-[1-(2,3-dioleoyloxy)propyl]-N,N,N-trimethylammonium (DOTAP), N-[1-(2,3-dioleyloxy)propyl]-N,N,N-trimethylammonium (DOTMA), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-arginyl-2,3-L-diaminopropionic acid-N-palmityl-N-olelyl-amide trihydrochloride (AtuFECT01), N,N-dimethyl-3-aminopropane derivatives [e.g. 1,2-distearoyloxy-N,N-dimethyl-3-aminopropane (DSDMA), 1,2-dioleyloxy-N,N-dimethyl-3-aminopropane (DoDMA), 1,2-dilinoleyloxy-N,N-3-dimethylaminopropane (DLinDMA), 2,2-dilinoleyl-4-dimethylaminomethyl [1,3]-dioxolane (DLin-K-DMA), phosphatidylserine derivatives [1,2-dioleyl-sn-glycero-3-phospho-L-serine, sodium salt (DOPS)], cholesterol}proteins (e.g. albumin, gelatins, atellocollagen), and peptides (e.g. protamine, PepFects, NickFects, polyarginine, polylysine, CADY, MPG).

Another preferred composition may comprise at least one excipient categorized as a second type of excipient. A second type of excipient may comprise or contain a conjugate group as described herein to enhance targeting and/or delivery of the composition and/or of the oligonucleotide of the invention to a tissue and/or cell and/or into a tissue and/or cell, as for example muscle or neuronal tissue or cell. Both types of excipients may be combined together into one single composition as identified herein.

The skilled person may select, combine and/or adapt one or more of the above or other alternative excipients and delivery systems to formulate and deliver a compound for use in the present invention.

Such a pharmaceutical composition of the invention may be administered in an effective concentration at set times to an animal, preferably a mammal. More preferred mammal is a human being. An oligonucleotide or a composition as defined herein for use according to the invention may be suitable for direct administration to a cell, tissue and/or an organ in vivo of individuals affected by or at risk of developing a disease or condition as identified herein, and may be administered directly in vivo, ex vivo or in vitro. Administration may be via systemic and/or parenteral routes, for example intravenous, subcutaneous, intraventricular, intrathecal, intramuscular, intranasal, enteral, intravitreal, intracerebral, epidural or oral route.

Preferably, such a pharmaceutical composition of the invention may be encapsulated in the form of an emulsion, suspension, pill, tablet, capsule or soft-gel for oral delivery, or in the form of aerosol or dry powder for delivery to the respiratory tract and lungs. In an embodiment an oligonucleotide of the invention may be used together with another compound already known to be used for the treatment of said disease. Such other compounds may be used for slowing down progression of disease, for reducing abnormal behaviors or movements, for reducing muscle tissue inflammation, for improving muscle fiber and/or neuronal function, integrity and/or survival and/or improve, increase or restore cardiac function. Examples are, but not limited to, a steroid, preferably a (gluco)corticosteroid, an ACE inhibitor (preferably perindopril), an angiotensin II type 1 receptor blocker (preferably losartan), a tumor necrosis factor-alpha (TNFα) inhibitor, a TGF□ inhibitor (preferably decorin), human recombinant biglycan, a source of mIGF-1, a myostatin inhibitor, mannose-6-phosphate, dantrolene, halofuginone, an antioxidant, an ion channel inhibitor, a protease inhibitor, a phosphodiesterase inhibitor (preferably a PDE5 inhibitor, such as sildenafil or tadalafil, and/or PDE10A inhibitors and/or MP-10), L-arginine, dopamine blockers, amantadine, tetrabenazine, co-enzyme Q10, antidepressants, anti-psychotics, anti-epileptics, mood-stabilizers in general, omega-3-fatty acids, creatine monohydrate, KMO inhibitors (Kynurenine mono oxigenase) such as CHDI246, or HDAC4 inhibitors such as PBT2. Such combined use may be a sequential use: each component is administered in a distinct composition. Alternatively each compound may be used together in a single composition.

### Use

In a further aspect, there is provided the use of a composition or an oligonucleotide as described in the previous sections for use as a medicament or part of therapy, or applications in which said oligonucleotide exerts its activity intracellularly.

Preferably, an oligonucleotide or composition of the invention is for use as a medicament or part of a therapy for preventing, delaying, curing, ameliorating and/or treating a human cis-element repeat instability associated genetic disorder. A human cis-element repeat instability associated genetic disorder is preferably a neuromuscular genetic disorder, more preferably as identified earlier herein.

### Method

In a further aspect, there is provided a method for preventing, treating, curing, ameliorating and/or delaying a condition or disease as defined in the previous section in an individual, in a cell, tissue or organ of said individual. The method comprising administering an oligonucleotide or a composition of the invention to said individual or a subject in the need thereof.

The method according to the invention wherein an oligonucleotide or a composition as defined herein may be suitable for administration to a cell, tissue and/or an organ in vivo of individuals affected by any of the herein defined diseases or at risk of developing said disease, and may be administered in vivo, ex vivo or in vitro. An individual or a subject in need is preferably a mammal, more preferably a human being.

In a further aspect, there is provided a method for diagnosis wherein the oligonucleotide of the invention is provided with a radioactive label or fluorescent label. In this method, an oligonucleotide of the invention may be used as an in situ probe to detect foci (RNA/protein aggregates resulting from the repeat expansion) in a sample from a subject. Said sample comprises cells from said subject.

In an embodiment, in a method of the invention, a concentration of an oligonucleotide or composition is ranged from 0.01 nM to 1 μM. More preferably, the concentration used is from 0.05 to 500 nM, or from 0.1 to 500 nM, or from 0.02 to 500 nM, or from 0.05 to 500 nM, even more preferably from 1 to 200 nM.

Dose ranges of an oligonucleotide or composition according to the invention are preferably designed on the basis of rising dose studies in clinical trials (in vivo use) for which rigorous protocol requirements exist. An oligonucleotide as defined herein may be used at a dose which is ranged from 0.01 to 200 mg/kg or 0.05 to 100 mg/kg or 0.1 to 50 mg/kg or 0.1 to 20 mg/kg, preferably from 0.5 to 10 mg/kg.

Dose ranges of an oligonucleotide or composition according to the invention may also be used at a dose which is
Ranged from 100 to 300 □g/week, 8 to 12 injections in total or
Ranged from 150 to 250 □g/week, 9 to 11 injections in total or
200 □g/week, 11 injections in total or
Ranged from 10 to 350 □g/day during two weeks or
Ranged from 50 to 250 □g/day during two weeks or
Ranged from 100 to 200 □g/day during two weeks or
Ranged from 20 to 80 □g/day during two weeks or
Ranged from 200 to 320 □g/day during two weeks or
320 □g/day, during two weeks or
30 □g/day, during two weeks.

The ranges of concentration or dose of oligonucleotide or composition as given above are preferred concentrations or doses for in vitro or ex vivo uses. The skilled person will understand that depending on the identity of the oligonucleotide used, the target cell to be treated, the gene target and its expression levels, the medium used and the transfection and incubation conditions, the concentration or dose of oligonucleotide used may further vary and may need to be optimised any further.

In this document and in its claims, the verb “to comprise” and its conjugations is used in its non-limiting sense to mean that items following the word are included, but items not specifically mentioned are not excluded. The verb “to comprise” is synonymous with the verb “to have” unless otherwise indicated. In addition the verb “to consist” may be replaced by “to consist essentially of” meaning that an oligonucleotide or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

### Definitions

Throughout the application, the word “binds”, “targets”, “hybridizes” could be used interchangeably when used in the context of an antisense oligonucleotide which is reverse complementary to a part of a pre-mRNA as identified herein. In the context of the invention, “hybridizes” or “binds” is used under physiological conditions in a cell, preferably a human cell unless otherwise indicated.

As used herein, “hybridization” refers to the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymine, 5-methyluracil and uracil which pair through the formation of hydrogen bonds. The natural base guanine is nucleobase complementary to the natural bases cytosine and 5-methyl-cytosine. Hybridization can occur under varying circumstances. In particular, hybridization of an oligonucleotide of the invention with a targeted pre-mRNA can occur under varying circumstances. Similarly, binding of an oligonucleotide of the invention to a targeted pre-mRNA can occur under varying circumstances. Preferably, said hybridization or said binding is assessed under physiological conditions in a cell, more preferably in a human cell. An oligonucleotide of the invention is preferably said to be able to bind to, or capable of binding to, or able to hybridize with, or capable of hybridizing with, when said binding or hybridization occurs under physiological conditions in a cell, preferably a human cell.

As used herein, “nucleotide” refers to a nucleoside further comprising a modified or unmodified phosphate linking group or a non-phosphate internucleoside linkage.

As used herein, “nucleotide analogue” or “nucleotide equivalent” refers to a nucleotide, which comprises at least one modification with respect to the nucleotides naturally occurring in RNA, such as A, C, G and U. Such a modification may be an internucleoside linkage modification and/or a sugar modification and/or a base modification.

As used herein, “monomer” refers to a precursor in the synthesis of an oligomeric or polymeric compound. Also the monomeric unit or residue within such an oligomeric or polymeric compound is encompassed in the term “monomer”. Thus, “monomer” and “nucleotide residue” may be used interchangeably throughout the description. Within the context of the present invention, a monomer is preferably a nucleotide. Preferred monomers to be incorporated in the oligonucleotides according to the invention are nucleotides comprising a 2′-O-methyl substituent, a phosphorothioate internucleoside linkage and a 5-methylpyrimidine and/or a 2,6-diaminopurine nucleobase.

As used herein, “nucleobase” refers to the heterocyclic base portion of a nucleoside. Nucleobases may be naturally occurring or may be modified and therefore include, but are not limited to adenine, cytosine, guanine, uracil, thymine and analogues thereof such as 5-methyl-cytosine. In certain embodiments, a nucleobase may comprise any atom or group of atoms capable of hydrogen bonding to a base of another nucleic acid. As used herein, “Tₘ” means melting temperature which is the temperature at which the two strands of a duplex nucleic acid separate. Tₘ is often used as a measure of duplex stability or the binding affinity of an antisense compound toward a complementary RNA molecule.

As used herein, “2′-modified” or “2′-substituted” refers to a nucleoside comprising a pentose sugar comprising a substituent at the 2′ position other than H or OH. 2′-modified nucleosides include, but are not limited to, bicyclic nucleosides wherein the bridge connecting two carbon atoms of the sugar ring connects the 2′ carbon and another carbon of the sugar ring; and nucleosides with non-bridging 2′-substituents, such as allyl, amino, azido, thio, O-allyl, O-C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), wherein each Rₘ and Rₙ is, independently, H or substituted or unsubstituted C₁-C₁₀ alkyl. 2′-modifed nucleosides may further comprise other modifications, for example at other positions of the sugar and/or at the nucleobase.

As used herein, “2′-O-Me”, “2′-OMe” or “2′-OCH3” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃-” or “2′-O-methoxyethyl” each refers to a nucleoside comprising a sugar comprising a —OCH₂CH₂OCH₃ group at the 2′ position of the sugar ring.

As used herein, the term “adenine analogue” means a chemically-modified purine nucleobase that, when incorporated into an oligomer, is capable of forming a base pair with either a thymine or uracil of a complementary strand of RNA or DNA. Preferably, such base pair is a Watson-Crick base pair, but analogues and slight deviations thereof are also considered allowable within the context of the present invention.

As used herein, the term “uracil analogue” means a chemically-modified pyrimidine nucleobase that, when incorporated into an oligomer, is capable of forming a base pair with either a adenine of a complementary strand of RNA or DNA. Preferably, such base pair is a Watson-Crick base pair, but analogues and slight deviations thereof are also considered allowable within the context of the present invention.

As used herein, the term “thymine analogue” means a chemically-modified pyrimidine nucleobase that, when incorporated into an oligomer, is capable of forming a base pair with an adenine of a complementary strand of RNA or DNA. Preferably, such base pair is a Watson-Crick base pair, but analogues and slight deviations thereof are also considered allowable within the context of the present invention.

As used herein, the term “cytosine analogue” means a chemically-modified pyrimidine nucleobase that, when incorporated into an oligomer, is capable of forming a base pair with a guanine of a complementary strand of RNA or DNA. For example, cytosine analogue can be a 5-methylcytosine. Preferably, such base pair is a Watson-Crick base pair, but analogues and slight deviations thereof are also considered allowable within the context of the present invention.

As used herein, the term “guanine analogue” means a chemically-modified purine nucleobase that, when incorporated into an oligomer, is capable of forming a base pair with a cytosine of a complementary strand of RNA or DNA. Preferably, such base pair is a Watson-Crick base pair, but analogues and slight deviations thereof are also considered allowable within the context of the present invention.

As used herein, the term “guanosine” refers to a nucleoside or sugar-modified nucleoside comprising a guanine or guanine analog nucleobase.

As used herein, the term “uridine” refers to a nucleoside or sugar-modified nucleoside comprising a uracil or uracil analog nucleobase.

As used herein, the term “thymidine” refers to a nucleoside or sugar-modified nucleoside comprising a thymine or thymine analog nucleobase.

As used herein, the term “cytidine” refers to a nucleoside or sugar-modified nucleoside comprising a cytosine or cytosine analog nucleobase.

As used herein, the term “adenosine” refers to a nucleoside or sugar-modified nucleoside comprising an adenine or adenine analog nucleobase.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein, “internucleoside linkage” refers to a covalent linkage between adjacent nucleosides. An internucleoside linkage may be a naturally occurring internucleoside linkage, i.e. a 3′ to 5′ phosphodiester linkage, or a modified internucleoside linkage.

As used herein, “modified internucleoside linkage” refers to any internucleoside linkage other than a naturally occurring internucleoside linkage.

As used herein, “backbone” refers to the chain of alternating sugar moieties and internucleoside linkages, as it occurs in an oligonucleotide. The oligonucleotide of the invention comprises at least one phosphorodithioate internucleoside linkage, but it has to be understood that more backbone modifications, such as sugar modifications and/or internucleoside linkage modifications may be present in the backbone.

As used herein, “oligomeric compound” refers to a polymeric structure comprising two or more sub-structures. In certain embodiments, an oligomeric compound is an oligonucleotide. In certain embodiments, an oligomeric compound is a single-stranded oligonucleotide. In certain embodiments, an oligomeric compound is a double-stranded duplex comprising two oligonucleotides. In certain embodiments, an oligomeric compound is a single-stranded or double-stranded oligonucleotide comprising one or more conjugate groups and/or terminal groups.

As used herein, “conjugate” refers to an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleoside of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “antisense compound” refers to an oligomeric compound, at least a portion of which is at least partially complementary to, or at least partially directed to, a target nucleic acid to which it hybridizes and modulates the activity, processing or expression of said target nucleic acid.

As used herein, “expression” refers to the process by which a gene ultimately results in a protein. Expression includes, but is not limited to, transcription, splicing, post-transcriptional modification, and translation.

As used herein, “antisense oligonucleotide” refers to an antisense compound that is an oligonucleotide.

As used herein, “antisense activity” refers to any detectable and/or measurable activity attributable to the hybridization of an anti sense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. In certain embodiments, antisense activity is assessed by observing a phenotypic change in a cell or animal.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is miRNA, mRNA, pre-mRNA, non-coding RNA, or natural antisense transcripts. For example, the target nucleic acid can be a cellular gene (or mRNA transcribed from the gene) whose expression is associated with a particular disorder or disease state,

As used herein, “target mRNA” refers to a pre-selected RNA molecule that encodes a protein.

As used herein, “targeting” or “targeted to” refers to the association of an antisense compound to a particular target nucleic acid molecule or a particular region of nucleotides within a target nucleic acid molecule. An antisense compound targets a target nucleic acid if it is sufficiently reverse complementary to the target nucleic acid to allow hybridization under physiological conditions. In this context “sufficiently reverse complementary” may be at least 90%, 95%, 97%, 99% or 100% reverse complementary with said targeted nucleic acid molecule.

As used herein, “target site” refers to a region of a target nucleic acid that is bound by an antisense compound. In certain embodiments, a target site is at least partially within the 3′ untranslated region of an RNA molecule. In certain embodiments, a target site is at least partially within the 5′ untranslated region of an RNA molecule. In certain embodiments, a target site is at least partially within the coding region of an RNA molecule. In certain embodiments, a target site is at least partially within an exon of an RNA molecule. In certain embodiments, a target site is at least partially within an intron of an RNA molecule. In certain embodiments, a target site is at least partially within a miRNA target site of an RNA molecule. In certain embodiments, a target site is at least partially within a repeat region of an RNA molecule.

As used herein, “target protein” refers to a protein, the expression of which is modulated by an antisense compound. In certain embodiments, a target protein is encoded by a target nucleic acid. In certain embodiments, expression of a target protein is otherwise influenced by a target nucleic acid.

As used herein, “complementarity” in reference to nucleobases refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases refers to a pair of nucleobases that do not form hydrogen bonds with one another or otherwise support hybridization.

As used herein, “complementary” in reference to linked nucleosides, oligonucleotides, or nucleic acids, refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid or reverse complementarity to a target nucleic acid.

As used herein, “modulation” refers to a perturbation of amount or quality of a function or activity when compared to the function or activity prior to modulation. For example, modulation includes the change, either an increase (stimulation or induction) or a decrease (inhibition or reduction) in gene expression. As a further example, modulation of expression can include perturbing splice site selection of pre-mRNA processing, resulting in a change in the amount of a particular splice-variant present compared to conditions that were not perturbed. As a further example, modulation includes perturbing translation of a protein.

As used herein, “motif” refers to a pattern of modifications in an oligomeric compound or a region thereof. Motifs may be defined by modifications at certain nucleosides and/or at certain linking groups of an oligomeric compound.

As used herein, “the same modifications” refer to modifications relative to naturally occurring molecules that are the same as one another, including absence of modifications. Thus, for example, two unmodified DNA nucleoside have “the same modification,” even though the DNA nucleoside is unmodified.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “pharmaceutically acceptable salts” refers to salts of active compounds that retain the desired biological activity of the active compound and do not impart undesired toxicological effects thereto.

As used herein, the term “independently” means that each occurrence of a repetitive variable within a claimed oligonucleotide is selected independent of one another. For example, each repetitive variable can be selected so that (i) each of the repetitive variables are the same, (ii) two or more are the same, or (iii) each of the repetitive variables can be different.

### General Chemistry Definitions

As used herein, “alkyl” refers to a saturated straight or branched hydrocarbon substituent or radical, typically containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to 24 carbon atoms, more typically from 1 to 12 carbon atoms (C₁-C₁₂ alkyl) with from 1 to 6 carbon atoms (C₁-C₆ alkyl) being more preferred. The term “lower alkyl” as used herein includes from 1 to 6 carbon atoms (C₁-C₆ alkyl). Alkyl groups as used herein may optionally contain one or more further substituents.

As used herein, “alkenyl” refers to a straight or branched hydrocarbon chain radical or substituent, typically containing up to twenty four carbon atoms, and having at least one carbon-carbon double bond. Examples of alkenyl groups include, but are not limited to, ethenyl, propenyl, butenyl, 1-methyl-2-buten-1-yl, dienes such as 1,3-butadienyl and the like. Alkenyl groups typically include from 2 to 24 carbon atoms, more typically from 2 to 12 carbon atoms with from 2 to 6 carbon atoms being more preferred. Alkenyl groups as used herein may optionally contain one or more further substituents.

As used herein, “alkynyl” refers to a straight or branched hydrocarbon radical or substituent, typically containing up to twenty four carbon atoms, and having at least one carbon-carbon triple bond. Examples of alkynyl groups include, but are not limited to, ethynyl, 1-propynyl, 1-butynyl, and the like. Alkynyl groups typically include from 2 to 24 carbon atoms, more typically from 2 to 12 carbon atoms with from 2 to 6 carbon atoms being more preferred. Alkynyl groups as used herein may optionally contain one or more further substituents.

As used herein, “aminoalkyl” refers to an amino substituted alkyl radical or substituent. This term is meant to include C₁-C₁₂ alkyl groups having an amino substituent at any position and wherein the aminoalkyl group is attached to the parent molecule via its alkyl moiety. The alkyl and/or amino portions of the aminoalkyl group may optionally be further substituted with further substituents.

As used herein, “aliphatic” refers to a straight or branched hydrocarbon radical or substituent, typically containing up to twenty four carbon atoms, wherein the saturation between any two carbon atoms is a single, double or triple bond. An aliphatic group preferably contains from 1 to 24 carbon atoms, more typically from 1 to 12 carbon atoms with from 1 to 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines. Aliphatic groups as used herein may optionally contain further substituents.

As used herein, “alicyclic” or “alicyclyl” refers to a cyclic radical or substituent, wherein the ring system is aliphatic. The ring system can comprise one or more rings wherein at least one ring is aliphatic. Preferred alicyclic moieties include rings having from 5 to 9 carbon atoms in the ring. Alicyclic groups as used herein may optionally contain further substituents.

As used herein, “alkoxy” refers to a radical or substituent comprising an alkyl group and an oxygen atom, wherein the alkoxy group is attached to a parent molecule via its oxygen atom. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally contain further substituents.

As used herein, “halo”, “halide” and “halogen” refer to an atom, radical or substituent selected from fluorine, chlorine, bromine and iodine.

As used herein, “aryl” and “aromatic” refer to a radical or substituent comprising a mono- or polycyclic carbocyclic ring system having one or more aromatic rings. Examples of aryl groups include, but are not limited to, phenyl, naphthyl, tetrahydronaphthyl, indanyl, idenyl and the like. Preferred aryl ring systems have from 5 to 20 carbon atoms in one or more rings. Aryl groups as used herein may optionally contain further substituents.

As used herein, “aralkyl” and “arylalkyl” refer to a radical or substituent comprising an alkyl group and an aryl group, wherein the aralkyl or arylalkyl group is attached to a parent molecule via its alkyl moiety. Examples include, but are not limited to, benzyl, phenethyl and the like. Aralkyl groups as used herein may optionally contain further substituents attached to the alkyl, the aryl or both moieties that form the radical or substituent.

As used herein, “heterocyclyl” refers to a radical or substituent comprising a mono- or polycyclic ring system that includes at least one heteroatom and is unsaturated, partially saturated or fully saturated, thereby including heteroaryl groups. Heterocyclyl is also meant to include fused ring system moieties wherein one or more of the fused rings contain at least one heteroatom and the other rings can contain one or more heteroatoms or optionally contain no heteroatoms. A heterocyclic group typically includes at least one atom selected from sulfur, nitrogen or oxygen. Examples of heterocyclic groups include [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally contain further substituents.

As used herein, “heteroaryl” and “heteroaromatic” refer to a radical or substituent comprising a mono- or polycyclic aromatic ring, ring system or fused ring system wherein at least one of the rings is aromatic and includes one or more heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals or substituents can be attached to a parent molecule directly or through a linking moiety such as an aliphatic group or a heteroatom. Heteroaryl groups as used herein may optionally contain further substituents.

As used herein, “heteroarylalkyl” refers to a radical or substituent comprising a heteroaryl group as previously defined and an alkyl moiety, wherein the heteroarylalkyl group is attached to a parent molecule via its alkyl moiety. Examples include, but are not limited to, pyridinylmethyl, pyrimidinylethyl, napthyridinylpropyl and the like. Heteroarylalkyl groups as used herein may optionally contain further substituents on one or both of the heteroaryl or alkyl portions.

As used herein, “mono or polycyclic” refers to any ring systems, such as a single ring or a polycyclic system having rings that are fused or linked, and is meant to be inclusive of single and mixed ring systems individually selected from aliphatic, alicyclic, aryl, heteroaryl, aralkyl, arylalkyl, heterocyclic, heteroaryl, heteroaromatic and heteroarylalkyl. Such mono and polycyclic structures can contain rings that have a uniform or varying degree of saturation, including fully saturated, partially saturated or fully unsaturated rings. Each ring can comprise ring atoms selected from C, N, O and S to give rise to heterocyclic rings as well as rings comprising only C ring atoms. Heterocyclic and all-carbon rings can be present in a mixed motif, such as for example benzimidazole wherein one ring of the fused ring system has only carbon ring atoms and the other ring has two nitrogen atoms. The mono or polycyclic structures can be further substituted with substituents such as for example phthalimide which has two oxo groups (═O) attached to one of the rings. In another aspect, mono or polycyclic structures can be attached to a parent molecule directly through a ring atom, through a substituent or a bifunctional linking moiety.

As used herein, “acyl” refers to a radical or substituent comprising a carbonyl moiety (C═O or —C(O)—) and a further substituent X, wherein the acyl group is attached to a parent molecule via its carbonyl moiety. As such, an acyl group is formally obtained by removal of a hydroxyl group from an organic acid and has the general formula —C(O)— X, wherein X is typically aliphatic, alicyclic or aromatic. The term “acyl” is also meant to include heteroacyl radicals or substituents with general formula —Y(O)ₙ—X, wherein X is as defined above and Y(O)ₙ is typically sulfonyl, sulfinyl or phosphate. Examples of acyl groups include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally contain further substituents.

As used herein, “substituent” and “substituent group” include groups that are typically added to other substituents or parent compounds to enhance desired properties or give desired effects. Substituent groups can be protected or unprotected and can be attached to one available site or to many available sites in a parent compound. Substituent groups may also be further substituted with other substituent groups and may be attached directly or via a linking group such as an alkyl or hydrocarbyl group to a parent compound. Herein, “hydrocarbyl” refers to any group comprising C, O and H. Included are straight, branched and cyclic groups having any degree of saturation. Such hydrocarbyl groups can include one or more heteroatoms selected from N, O and S and can be further substituted with one or more substituents.

Unless otherwise indicated, the term “substituted” or “optionally substituted” refers to the (optional) presence of any of the following substituents: halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxo (—O—Rₐₐ), aryl, aralkyl, heterocyclic, heteroaryl, heteroarylalkyl, amino (—NR_{bb}R_{cc}), imino (↑NR_{bb}), amido (—C(O)NR_{bb}R_{cc} or —N(R_{bb})C(O)Rₐₐ), azido (—N₃), nitro (—NO₂), cyano (—CN), carbamido (—OC(O)NR_{bb}R_{cc}or —N(R_{bb})C(O)ORₐₐ), ureido (—N(R_{bb})C(O)NR_{bb}R_{cc}), thioureido (—N(R_{bb})C(S)NR_{bb}R_{cc}), guanidinyl (—N(R_{bb})C(═NR_{bb})NR_{bb}R_{cc}), amidinyl (—C(═NR_{bb})NR_{bb}R_{cc} or —N(R_{bb})C(NR_{bb})Rₐₐ), thiol (—SR_{bb}), sulfinyl (—S(O)R_{bb}), sulfonyl (—S(O)₂R_{bb}), sulfonamidyl (—S(O)₂NR_{bb}R_{cc} or —N(R_{bb})S(O)₂R_{bb}) and conjugate groups. Herein, each Rₐₐ, R_{bb} and R_{cc} is, independently, H, an optionally linked chemical functional group or a further substituent, preferably but without limitation chosen from the group consisting of H, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by way of example and not limitation, physical properties such as molecular weight, solubility or log P, application properties such as activity against the intended target and practical properties such as ease of synthesis.

Recursive substituents are an intended aspect of the invention. One of ordinary skill in the art of medicinal and organic chemistry understands the versatility of such substituents. To the degree that recursive substituents are present in a claim of the invention, the total number will be determined as set forth above.

As used herein, a zero (0) in a range indicating number of a particular unit means that the unit may be absent. For example, an oligomeric compound comprising 0-2 regions of a particular motif means that the oligomeric compound may comprise one or two such regions having the particular motif, or the oligomeric compound may not have any regions having the particular motif. In instances where an internal portion of a molecule is absent, the portions flanking the absent portion are bound directly to one another. Likewise, the term “none” as used herein, indicates that a certain feature is not present. As used herein, “analogue” or “derivative” means either a compound or moiety similar in structure but different in respect to elemental composition from the parent compound regardless of how the compound is made. For example, an analogue or derivative compound does not need to be made from the parent compound as a chemical starting material.

The following examples are offered for illustrative purposes only, and are not intended to limit the scope of the present invention in any way.

### BRIEF DESCRIPTION OF THE FIGURES

FIGS. 1A-1B. In vitro activity assay for (XYG)₇ in which X=5-methylcytosine and Y=U (PS659 SEQ ID NO:90; derived from SEQ ID NO:2) and (XYG)₇ in which X=C and Y is 5-methyluracil (PS661 SEQ ID NO:97; derived from SEQ ID NO:3). PS659 (FIG. 1A) and PS661 (FIG. 1B) were transfected into HD fibroblasts (GM04022) at increasing concentrations (0.5-200 nM). Efficacy and selectivity was determined with RT-PCR and lab-on-a-chip analysis. Silencing of the expanded ((CAG)₄₄) and healthy ((CAG)₁₈) HTT transcripts were compared to the relative HTT transcript levels in mock samples. For all AONs n=2 except for mock (n=3).

FIGS. 2A-2D. In vivo efficacy of PS659 ((XYG)₇ in which X=5-methylcytosine and Y=U; SEQ ID NO:2) in a transgenic HD rat model. Transgenic HD rats ((CAG)₅₁ repeat) received 15 times an intraventricular injection with PS659 (SEQ ID NO:90 derived from SEQ ID NO:2), during 18 weeks at a final dose of 200 μg per injection, control HD rats received vehicle only. Rats were sacrificed one week after the final injection. From all rats tissue was isolated and HTT levels were determined with Q-RT-PCR analysis. Reduced levels of HTT transcript were found in (FIG. 2A) cortex, (FIG. 2B) hippocampus, (FIG. 2C) olfactory bulb and (FIG. 2D) thalamus after PS659 treatment compared to control.

TABLE 1
General structures of AONs.

| Target Repeat | AON Sequence (5′ → 3′) | SEQ ID NO |
|---|---|---|
| (CAG)n | (XYG)7 (PS57) | 1 |
| | X = C, Y = U | |
| | (XYG)7 (PS659) | 2 |
| | X = 5-methylcytosine, Y = U | |
| | (XYG)7 (PS661) | 3 |
| | X = C, Y = 5-methyluracil | |
| | (XYG)4 | 4 |
| | (XYG)5 | 5 |
| | (XYG)6 | 6 |
| | (XYG)7 | 7 |
| | (XYG)8 | 8 |
| | (XYG)9 | 9 |
| | (XYG)10 | 10 |
| | (XYG)11 | 11 |
| | (XYG)12 | 12 |
| (GCG)n | (XGX)4 | 13 |
| | (XGX)5 | 14 |
| | (XGX)6 | 15 |
| | (XGX)7 | 16 |
| | (XGX)8 | 17 |
| | (XGX)9 | 18 |
| | (XGX)10 | 19 |
| | (XGX)11 | 20 |
| | (XGX)12 | 21 |
| (CGG)n | (XXG)4 | 22 |
| | (XXG)5 | 23 |
| | (XXG)6 | 24 |
| | (XXG)7 | 25 |
| | (XXG)8 | 26 |
| | (XXG)9 | 27 |
| | (XXG)10 | 28 |
| | (XXG)11 | 29 |
| | (XXG)12 | 30 |
| (GAA)n | (YYX)4 | 31 |
| | (YYX)5 | 32 |
| | (YYX)6 | 33 |
| | (YYX)7 | 34 |
| | (YYX)8 | 35 |
| | (YYX)9 | 36 |
| | (YYX)10 | 37 |
| | (YYX)11 | 38 |
| | (YYX)12 | 39 |
| (GCC)n | (GGX)4 | 40 |
| | (GGX)5 | 41 |
| | (GGX)6 | 42 |
| | (GGX)7 | 43 |
| | (GGX)8 | 44 |
| | (GGX)9 | 45 |
| | (GGX)10 | 46 |
| | (GGX)11 | 47 |
| | (GGX)12 | 48 |
| (CCG)n | (XGG)4 | 49 |
| | (XGG)5 | 50 |
| | (XGG)6 | 51 |
| | (XGG)7 | 52 |
| | (XGG)8 | 53 |
| | (XGG)9 | 54 |
| | (XGG)10 | 55 |
| | (XGG)11 | 56 |
| | (XGG)12 | 57 |
| (AUUCU)n | (ZGZZY)3 | 58 |
| | (ZGZZY)4 | 59 |
| | (ZGZZY)5 | 60 |
| | (ZGZZY)6 | 61 |
| | (ZGZZY)7 | 62 |
| (CCUG)n | (XZGG)3 | 63 |
| | (XZGG)4 | 64 |
| | (XZGG)5 | 65 |
| | (XZGG)6 | 66 |
| | (XZGG)7 | 67 |
| | (XZGG)8 | 68 |
| | (XZGG)9 | 69 |
| (GGGGCC)n | (GGXUXX)3 | 216 |
| | (GGXUXX)4 | 217 |
| | (GGXIXX)4 | 218 |
| | (GGXQXX)4 | 219 |

X = C or 5-methylcytosine
Y = U or 5-methyluracil,
Z = A or 2,6-diaminopurine,
I = inosine, and
Q = abasic monomer.
Note:
All AONs with SEQ ID NO: 4-69, or 216-219 comprise at least one base modification selected from 5-methylcytosine, 5-methyluracil, and 2,6-diaminopurine.

TABLE 2
General structures of AONs.

| Target | AON | | SEQ ID |
|---|---|---|---|
| Repeat | ID | AON Sequence (5′→3′) | NO |
| (CAG)n | PS659 | CUG CUG CUG CUG CUG CUG CUG | 90 |
| | | CUG CUG CUG CUG CUG CUG CUG | 91 |
| | | CUG CUG CUG CUG CUG CUG CUG | 92 |
| | | CUG CUG CUG CUG CUG CUG CUG | 93 |
| | | CUG CUG CUG CUG CUG CUG CUG | 94 |
| | | CUG CUG CUG CUG CUG CUG CUG | 95 |
| | | CUG CUG CUG CUG CUG CUG CUG | 96 |
| | PS661 | CUG CUG CUG CUG CUG CUG CUG | 97 |
| | | CUG CUG CUG CUG CUG CUG CUG | 98 |
| | | CUG CUG CUG CUG CUG CUG CUG | 99 |
| | | CUG CUG CUG CUG CUG CUG CUG | 100 |
| | | CUG CUG CUG CUG CUG CUG CUG | 101 |
| | | CUG CUG CUG CUG CUG CUG CUG | 102 |
| | | CUG CUG CUG CUG CUG CUG CUG | 103 |
| | PS660 | CUG CUG CUG CUG CUG CUG CUG | 104 |
| | | CUG CUG CUG CUG CUG CUG CUG | 105 |
| | | CUG CUG CUG CUG CUG CUG CUG | 106 |
| | PS684 | CUG CUG CUG CUG CUG CUG CUG | 107 |
| | | CUG CUG CUG CUG CUG CUG CUG QQQQ | 220 |
| | | CUG CUG CUG CUG CUG CUG CUG | 108 |
| | | CUG CUG CUG CUG CUG CUG CUG QQQQ | 221 |
| | | CUG CUG CUG CUG CUG CUG CUG CUG | 109 |
| | | CUG CUG CUG CUG CUG CUG CUG CUG | 110 |
| | | CUG CUG CUG CUG CUG CUG CUG CUG CUG | 111 |
| | | CUG CUG CUG CUG CUG CUG CUG CUG CUG | 112 |
| | | CUG CUG CUG CUG CUG CUG CUG CUG CUG CUG | 113 |
| | | CUG CUG CUG CUG CUG CUG CUG CUG CUG CUG | 114 |
| | | CUG CUG CUG CUG CUG CUG CUG CUG CUG CUG | 115 |
| | | CUG | |
| | | CUG CUG CUG CUG CUG CUG CUG CUG CUG CUG | 116 |
| | | CUG | |
| | | CUG CUG CUG CUG CUG CUG CUG CUG CUG CUG | 117 |
| | | CUG CUG | |
| | | CUG CUG CUG CUG CUG CUG CUG CUG CUG CUG | 118 |
| | | CUG CUG | |
| (GCG)n | | CGC CGC CGC CGC | 119 |
| | | CGC CGC CGC CGC | 120 |
| | | CGC CGC CGC CGC CGC | 121 |
| | | CGC CGC CGC CGC CGC | 122 |
| | | CGC CGC CGC CGC CGC CGC | 123 |
| | | CGC CGC CGC CGC CGC CGC | 124 |
| | | CGC CGC CGC CGC CGC CGC CGC | 125 |
| | | CGC CGC CGC CGC CGC CGC CGC | 126 |
| | | CGC CGC CGC CGC CGC CGC CGC CGC | 127 |
| | | CGC CGC CGC CGC CGC CGC CGC CGC | 128 |
| | | CGC CGC CGC CGC CGC CGC CGC CGC CGC | 129 |
| | | CGC CGC CGC CGC CGC CGC CGC CGC CGC | 130 |
| | | CGC CGC CGC CGC CGC CGC CGC CGC CGC CGC | 131 |
| | | CGC CGC CGC CGC CGC CGC CGC CGC CGC CGC | 132 |
| (CGG)n | | CCG CCG CCG CCG | 133 |
| | | CCG CCG CCG CCG | 134 |
| | | CCG CCG CCG CCG CCG | 135 |
| | | CCG CCG CCG CCG CCG | 136 |
| | | CCG CCG CCG CCG CCG CCG | 137 |
| | | CCG CCG CCG CCG CCG CCG | 138 |
| | | CCG CCG CCG CCG CCG CCG CCG | 139 |
| | | CCG CCG CCG CCG CCG CCG CCG | 140 |
| | | CCG CCG CCG CCG CCG CCG CCG CCG | 141 |
| | | CCG CCG CCG CCG CCG CCG CCG CCG | 142 |
| | | CCG CCG CCG CCG CCG CCG CCG CCG CCG | 143 |
| | | CCG CCG CCG CCG CCG CCG CCG CCG CCG | 144 |
| | | CCG CCG CCG CCG CCG CCG CCG CCG CCG CCG | 145 |
| | | CCG CCG CCG CCG CCG CCG CCG CCG CCG CCG | 146 |
| (GAA)n | | UUC UUC UUC UUC | 147 |
| | | UUC UUC UUC UUC | 148 |
| | | UUC UUC UUC UUC UUC | 149 |
| | | UUC UUC UUC UUC UUC | 150 |
| | | UUC UUC UUC UUC UUC UUC | 151 |
| | | UUC UUC UUC UUC UUC UUC | 152 |
| | | UUC UUC UUC UUC UUC UUC UUC | 153 |
| | | UUC UUC UUC UUC UUC UUC UUC | 154 |
| | | UUC UUC UUC UUC UUC UUC UUC | 155 |
| | | UUC UUC UUC UUC UUC UUC UUC | 156 |
| | | UUC UUC UUC UUC UUC UUC UUC | 157 |
| | | UUC UUC UUC UUC UUC UUC UUC UUC | 158 |
| | | UUC UUC UUC UUC UUC UUC UUC UUC | 159 |
| | | UUC UUC UUC UUC UUC UUC UUC UUC UUC | 160 |
| | | UUC UUC UUC UUC UUC UUC UUC UUC UUC | 161 |
| | | UUC UUC UUC UUC UUC UUC UUC UUC UUC UUC | 162 |
| | | UUC UUC UUC UUC UUC UUC UUC UUC UUC UUC | 163 |
| | | UUC UUC UUC UUC UUC UUC UUC UUC UUC UUC | 164 |
| | | UUC | |
| | | UUC UUC UUC UUC UUC UUC UUC UUC UUC UUC | 165 |
| | | UUC | |
| | | UUC UUC UUC UUC UUC UUC UUC UUC UUC UUC | 166 |
| | | UUC UUC | |
| | | UUC UUC UUC UUC UUC UUC UUC UUC UUC UUC | 167 |
| | | UUC UUC | |
| (GCC)n | | GGC GGC GGC GGC | 168 |
| | | GGC GGC GGC GGC GGC | 169 |
| | | GGC GGC GGC GGC GGC GGC | 170 |
| | | GGC GGC GGC GGC GGC GGC GGC | 171 |
| | | GGC GGC GGC GGC GGC GGC GGC | 172 |
| | | GGC GGC GGC GGC GGC GGC GGC | 173 |
| | | GGC GGC GGC GGC GGC GGC GGC | 174 |
| | | GGC GGC GGC GGC GGC GGC GGC GGC | 175 |
| | | GGC GGC GGC GGC GGC GGC GGC GGC GGC | 176 |
| | | GGC GGC GGC GGC GGC GGC GGC GGC GGC GGC | 177 |
| (CCG)n | | CGG CGG CGG CGG | 178 |
| | | CGG CGG CGG CGG CGG | 179 |
| | | CGG CGG CGG CGG CGG CGG | 180 |
| | | CGG CGG CGG CGG CGG CGG CGG | 181 |
| | | CGG CGG CGG CGG CGG CGG CGG CGG | 182 |
| | | CGG CGG CGG CGG CGG CGG CGG CGG CGG | 183 |
| | | CGG CGG CGG CGG CGG CGG CGG CGG CGG CGG | 184 |
| (AUUCU)n | | AGAAU AGAAU AGAAU | 185 |
| | | AGAAU AGAAU AGAAU AGAAU | 186 |
| | | AGAAU AGAAU AGAAU AGAAU | 187 |
| | | AGAAU AGAAU AGAAU AGAAU | 188 |
| | | AGAAU AGAAU AGAAU AGAAU | 189 |
| | | AGAAU AGAAU AGAAU AGAAU AGAAU | 190 |
| | | AGAAU AGAAU AGAAU AGAAU AGAAU AGAAU | 191 |
| | | AGAAU AGAAU AGAAU AGAAU AGAAU AGAAU | 192 |
| | | AGAAU | |
| (CCUG)n | | CAGG CAGG CAGG | 193 |
| | | CAGG CAGG CAGG | 194 |
| | | CAGG CAGG CAGG CAGG | 195 |
| | | CAGG CAGG CAGG CAGG | 196 |
| | | CAGG CAGG CAGG CAGG CAGG | 197 |
| | | CAGG CAGG CAGG CAGG CAGG | 198 |
| | | CAGG CAGG CAGG CAGG CAGG | 199 |
| | | CAGG CAGG CAGG CAGG CAGG | 200 |
| | | CAGG CAGG CAGG CAGG CAGG CAGG | 201 |
| | | CAGG CAGG CAGG CAGG CAGG CAGG | 202 |
| | | CAGG CAGG CAGG CAGG CAGG CAGG CAGG | 203 |
| | | CAGG CAGG CAGG CAGG CAGG CAGG CAGG | 204 |
| | | CAGG CAGG CAGG CAGG CAGG CAGG CAGG CAGG | 205 |
| | | CAGG CAGG CAGG CAGG CAGG CAGG CAGG CAGG | 206 |
| | | CAGG CAGG CAGG CAGG CAGG CAGG CAGG CAGG | 207 |
| | | CAGG | |
| | | CAGG CAGG CAGG CAGG CAGG CAGG CAGG CAGG | 208 |
| | | CAGG | |
| (GGGGCC)n | PS1252 | GGCUCC GGCUCC GGCUC | 209 |
| | | GGCQCC GGCQCC GGCQCC | 210 |
| | | GGCUCC GGCUCC GGCUCC | 211 |
| | | GGCUCC GGCUCC GGCUCC GGCUCC | 212 |
| | | GGCQCC GGCQCC GGCQCC GGCQCC | 213 |
| | | GGCICC GGCICC GGCICC GGCICC | 214 |
| | | GGCCUC GGCCUC GGCCUC GGCCUC | 215 |

All AONs are 2′-O-methyl phosphorothioate AONs wherein C is 5-methylcytosine, U is 5-methyluracil, A is 2,6-diaminopurine, I is inosine and Q is an abasic monomer.

### EXAMPLES

### Example 1

### Introduction

The particular characteristics of a chosen antisense oligonucleotide (AON) chemistry may at least in part enhance binding affinity and stability, enhance activity, improve safety, and/or reduce cost of goods by reducing length or improving synthesis and/or purification procedures. This example describes the comparative analysis of the activity of AONs designed to target the expanded (CAG), repeat in HTT transcripts in HD fibroblasts in vitro, and includes AONs with either 5-methylcytosines (XYG)₇, wherein X is 5-methylcytosine and Y=U being also identified as SEQ ID NO:90 (and derived from SEQ ID NO:2), or 5-methyluracils (XYG)₇, wherein X=C and Y=5-methyluracil being also identified as SEQ ID NO:97 (and derived from SEQ ID NO:3).

### Materials and Methods

Cell culture. Patient derived HD fibroblasts (GM04022) (purchased from Coriell Cell Repositories, Camden, USA) were cultured at 37° C. and 5% CO₂ in Minimal Essential Medium (MEM) (Gibco Invitrogen, Carlsbad, USA) with 15% heat inactivated Fetal Bovine Serum (FBS) (Clontech, Palo Alto USA), 1% Glutamax (Gibco) and 100 U/ml penicillin/streptomycin (P/S) (Gibco).

Oligonucleotides. The AONs were fully 2′-O-methyl phosphorothioate modified: PS659; (XYG)₇, wherein X is 5-methylcytosine and Y=U being also identified as SEQ ID NO:90 (and derived from SEQ ID NO:2), and PS661; (XYG)₇, wherein X=C and Y=5-methyluracil being also identified as SEQ ID NO:97 (and derived from SEQ ID NO:3).

Transfection. Cells were transfected with AONs complexed with PEI (2 μL per ug AON, in 0.15 M NaCl). AON-PEI complex was added in MEM medium with 5% FBS to cells to a final AON concentration varying from 0.5-200 nM. Fresh medium was supplemented after four hours and after 24 hours RNA was isolated.

RNA isolation. RNA from cultured cells was isolated using the Aurum Total RNA Mini Kit (Bio-Rad, Hercules, CA) according to the manufacturer's protocol.

RT-PCR and Lab-on-a-chip analysis. Approximately 200 ng RNA was subjected to cDNA synthesis with random hexamers using the SuperScript first-strand synthesis system (Invitrogen) in a total volume of 20 μL. PCR was performed with primers for HTT (across the CAG repeat) and β-actin. The PCR program started with a 4 min initial denaturation at 95° C., followed by 35 cycles of 30 sec denaturation at 94° C., 30 sec annealing at 60° C., 45 sec elongation at 72° C., after which a final elongation step was performed at 72° C. for 7 min. Lab-on-a-Chip was performed on the Agilent 2100 Bioanalyzer (Agilent Technologies, Waldbronn, Germany), using the Agilent DNA 1000 Kit. Expression levels were normalized for β-actin levels and relative to transcript levels without transfection. The following primers were used:

HTT forward;
(SEQ ID NO: 70)
5′-ATGGCGACCCTGGAAAAGCTGAT-3′
HTT reverse:
(SEQ ID NO: 71)
5′-TGAGGCAGCAGCGGCTG-3′
β-actin forward;
(SEQ ID NO: 72)
5′-GGACTTCGAGCAAGAGATGG-3′
β-actin reverse;
(SEQ ID NO: 73)
5′-AGCACTGTGTTGGCGTACAG-3′

### Results

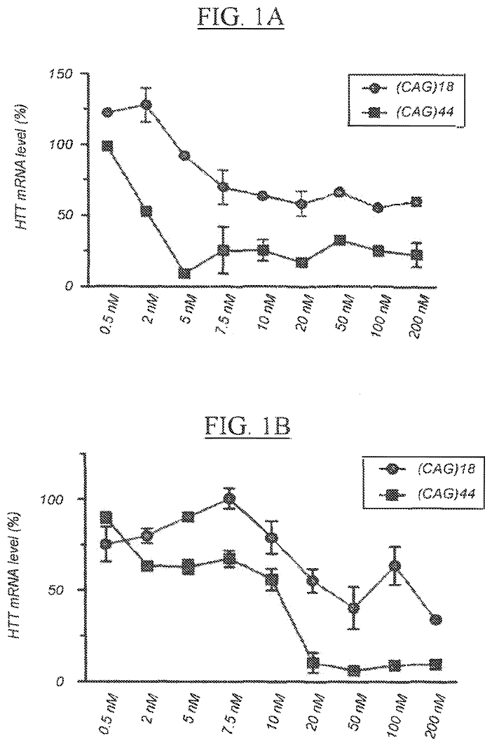

Both PS659 (SEQ ID NO:90 derived from SEQ ID NO:2) and PS661 (SEQ ID NO:97 derived from SEQ ID NO:3) were highly effective and reduced the HTT transcripts in HD fibroblasts in a dose-dependent manner (FIGS. 1A-1B). Both AONs also showed a preference for the allele with the expanded CAG repeats. PS659 (SEQ ID NO:90 derived from SEQ ID NO:2) was more effective and more allele-specific at lower concentrations (strongest effect at 5 nM) (FIG. 1A) than PS661 (SEQ ID NO:97 derived from SEQ ID NO:3) (strongest effect at 20 nM) (FIG. 1B).

### Example 2

### Introduction

PS659 (XYG)₇, wherein X is 5-methylcytosine and Y=U also identified as SEQ ID NO:90 (derived from SEQ ID NO:2), was selected from in vitro studies as most efficient and safe candidate. This example describes its activity in a transgenic HD rat model after a series of direct intraventricular injections.

### Materials and Methods

Animals. Transgenic HD rats carry a truncated Huntington cDNA fragment with 51 CAG repeats under the control of the native rat Huntington promoter. The expressed gene product is about 75 kDa, corresponding to 22% of the full-length Huntington (cDNA position 324-2321, amino acid position 1-709/825, corresponding to exon 1-16), under the control of 886 bp of the rat Huntington promoter (von Hörsten S. et al.). All animal experiments were approved by the Institutional Animal Care and Use Committees of the Maastricht University, Maastricht.

Oligonucleotides. PS659 (XYG)₇, wherein X is 5-methylcytosine and Y=U also identified as SEQ ID NO:90 (derived from SEQ ID NO:2), is a fully 2′-O-methyl phosphorothioate modified AON.

In vivo treatment. Transgenic HD rats received 15 times an intraventricular injection at a final dose of 200 μg PS659 also identified as SEQ ID NO:90 (derived from SEQ ID NO:2) during 18 weeks. Control HD rats received vehicle only. Rats were sacrificed one week after the final injection.

RNA isolation. RNA from brain tissue was isolated using RNA-Bee reagent (Tel Test, Inc). In brief, tissue samples were homogenized in MagNA Lyser green bead tubes (Roche) by adding RNA-Bee (50 mg tissue/mL RNA-Bee) and homogenizing using a MagNA Lyser instrument (Roche). Lysate was transferred to a new tube, chloroform (SIGMA) was added (0.2 mL per mL RNA-Bee), mixed, incubated on ice for 5 minutes and centrifuged at 13,000 rpm for 15 minutes at 4° C. The upper aqueous phase was collected and an equal volume isopropanol (SIGMA) was added, followed by a 1 hour incubation period at 4° C. and centrifugation (13,000 rpm, 15 min, 4° C.). The RNA precipitate was washed with 70% (v/v) ethanol (BioSolve), air dried and dissolved in MilliQ.

Quantitative RT-PCR analysis. Approximately 200 ng was subjected to cDNA synthesis with random hexamers using the SuperScript first-strand synthesis system (Invitrogen) in a total volume of 20 μL. 3 μL of 1/40 cDNA dilution preparation was subsequently used in a quantitative PCR analysis according to standard procedures in presence of iQ™ SYBR® Green Supermix (Bio-Rad). Quantitative PCR primers were designed based on NCBI database sequence information. Product identity was confirmed by DNA sequencing. The signal for Rab2 and YWHAZ was used for normalization. The following primers were used:

Rat Htt-F;
(SEQ ID NO: 74)
5′-CGCCGCCTCCTCAGCTTC-3′
Rat Htt-R;
(SEQ ID NO: 75)
5′-GAGAGTTCCTTCTTTGGTCGGTGC-3′
Rab2-F;
(SEQ ID NO: 76)
5′-TGGGAAACAGATAAAACTCCAGA-3′
Rab2-R;
(SEQ ID NO: 77)
5′-AATATGACCTTGTGATAGAACGAAAG-3′
YWHAZ-F;
(SEQ ID NO: 78)
5′-AAATGAGCTGGTGCAGAAGG-3′
YWHAZ-R;
(SEQ ID NO: 79)
5′-GGCTGCCATGTCATCGTAT-3′

### Results

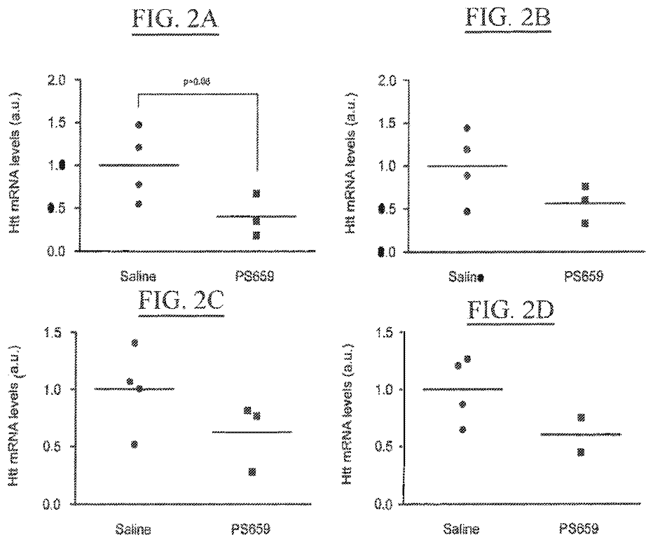

PS659 (also identified as SEQ ID NO:90 or derived from SEQ ID NO:2) reduced transgenic Htt transcript levels in cortex (FIG. 2A), hippocampus (FIG. 2B), olfactory bulb (FIG. 2C) as well as in thalamus (FIG. 2D) when compared to saline treated rats. These results demonstrate that PS659 (also identified as SEQ ID NO:90 or derived from SEQ ID NO; 2) is effective in vivo after direct intraventricular injection.

### List of references

Aartsma-Rus et al., Hum Mol Gen 2003; 12(8):907-14.
Arai K et al. Bioorg. Med. Chem. 2011, 21, 6285
Bauer et al., 2009; J Neurochem. 110:1737-65
Braida C., et al, Human Molecular Genetics, 2010, vol 9:1399-1412
Bruno et al., Adv Drug Deliv Rev. 2011; 63(13):1210-26
Diebold et al., 2006, Eur J Immunol; 36(12):3256-67
Evers et al. PLoS ONE 2011, 6(9)e24308
Huang et al., 1998 Somat Cell Molec Gen 24:217-33;
Krieg A M. et al., Nature 1995; 374:546-549.
Krieg, A. M., Curr. Opin. Immunol. 2000; 12:35-43.
Kumar L, Pharm. Technol. 2008, 3, 128.
Muchowski et al., 2002 PNAS 99:727-32
Mulders et al. PNAS 2009 106(33); p13915-20
Peacock H et al. J. Am. Chem. Soc. 2011, 133, 9200
Popovic P J. et al. J of Immunol 2006; 177:8701-8707.
Roon-Mom et al., 2002 Mol Brain Res 109:1-10
Ropper A H. and Brown R H., 2005 Principles of neurology. 8^{th} Ed. New York: McGraw-Hill, 2005.
Ross et al., 2011; Lancet Neurol. 10:83-98
Rigo, F, et al, 2012, Nature chemical biology, 8:555-561.
Steffan et al., 2000 PNAS 97:6763-68
von Hörsten S. et al. Hum Mol Genet. 2003; 12(6):617-24
Wagner, H., Adv. Immunol. 1999; 73:329-368.
Yu R Z., Anal Biochem 2002; 304:19-25.

1-15. (canceled)

16. An oligonucleotide comprising a 2′-O-methyl RNA nucleotide residue, having a backbone wherein at least one phosphate moiety is replaced by a phosphorothioate moiety further comprising at least one of a 5-methylpyrimidine bases and/or a 2,6-diaminopurine base, and comprising one or more abasic sites or monomers, wherein said oligonucleotide is able to hybridize to a repetitive element having a repetitive nucleotide unit of (CAG)n.

17. An oligonucleotide according to claim 16, wherein the oligonucleotide is represented by H—(P)ₚ—(R)r—(Q)_{q}—H, wherein:
H is a hydrogen atom,
(R)r is a repeating nucleotide unit, wherein R is CUG,
P or Q is an abasic site or monomer or a nucleotide A, C, G, U or an analogue or equivalent thereof, and
q or p is an integer.

18. An oligonucleotide according to claim 16, wherein the oligonucleotide is represented by:
H—(P′)p′UG—(CUG)m—CU(Q′)q′—H,
H—(P′)p″G—(CUG)m−CU(Q′)q′—H,
H—(P′)p′UG—(CUG)m—C(Q′)q″—H, or
H—(P′)p″G—(CUG)m—C(Q′)q″—H,
wherein both p and q are not 0, and both (P)p and (Q)q are represented by (P′)p′UG or (P′)p″G and CU(Q′)q′ or C(Q′)q″,
wherein each occurrence of P′ is, individually, an abasic site or a nucleotide, such as A, C, G, U or an analogue or equivalent thereof, and p′ is p−2 and p″ is p−1 and
each occurrence of Q′ is, individually, an abasic site or a nucleotide, such as A, C, G, U or an analogue or equivalent thereof, and q′ is q−2 and q″ is q−1.

19. An oligonucleotide according to claim 16, wherein the abasic site or monomer is within the base sequence of said oligonucleotide.

20. An oligonucleotide according to claim 16, wherein the length of said oligonucleotide is between 12 and 36 nucleotides or is less than 12 nucleotides.

21. An oligonucleotide according to claim 16, wherein said oligonucleotide comprises 1 to 10 abasic sites or monomers at a single terminus or at both termini of the oligonucleotide (5′ and 3′).

22. An oligonucleotide according to claim 16, wherein said oligonucleotide is a single stranded oligonucleotide.

23. An oligonucleotide according to claim 16, wherein said oligonucleotide is at least 90% reverse complementary with said repetitive nucleotide unit and/or remains in association to its target when there are up to 20% of mismatched nucleotides.

24. An oligonucleotide according to claim 16, wherein said oligonucleotide reduces a detectable amount of mutant transcript and/or reduces the translation of said mutant transcripts and thus the amount of mutant protein and/or induce the specific degradation of the expanded repeat transcripts.

25. An oligonucleotide according to claim 16, wherein the base sequence of said oligonucleotide comprises or consists of SEQ ID NO:107 or 118, and has a length from 24-36 nucleotides.

26. An oligonucleotide according to claim 16, wherein the base sequence of said oligonucleotide consists of SEQ ID NO:107 or 108, and said oligonucleotide is represented as SEQ ID NO:220 or 221.

27. A composition comprising an oligonucleotide as defined in claim 16.

28. A method for treating, delaying, ameliorating and/or preventing the human genetic diseases Huntington's disease (HD), spinocerebellar ataxia (SCA) type 1, 2, 3, 6, 7, 12 or 17, amyotrophic lateral sclerosis (ALS), frontotemporal dementia (FTD), X-linked spinal and bulbar muscular atrophy (SBMA) and/or dentatorubropallidoluysian atrophy (DRPLA) caused by CAG repeat expansions in the transcripts of the HTT (SEQ ID NO:80), ATXN1 (SEQ ID NO:81), ATXN2 (SEQ ID NO:82) ATXN3 (SEQ ID NO:83), CACNA1A (SEQ ID NO:84), ATXN7 (SEQ ID NO:85), PPP2R2B (SEQ ID NO:86), TBP (SEQ ID NO:87), AR (SEQ ID NO:88) or ATN1 (SEQ ID NO:89) genes in an individual or a subject in the need thereof, wherein said method comprises administering an oligonucleotide according to claim 16 to individual or a subject in the need thereof.

29. A method according to claim 28, wherein the administration of said oligonucleotide is via an intravenous, subcutaneous, intraventricular, intrathecal, intramuscular, intranasal, enteral, intravitreal, intracerebral, epidural or oral route.


SEQUENCE LISTING (221 sequences)

SEQ ID NO: 1 - RNA, 21 nt, synthetic construct
  source 1..21: mol_type = other RNA; note = Oligonucleotide
ctgctgctgctgctgctgctg

SEQ ID NO: 2 - RNA, 21 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  source 1..21: mol_type = other RNA; note = Oligonucleotide
ntgntgntgntgntgntgntg

SEQ ID NO: 3 - RNA, 21 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  source 1..21: mol_type = other RNA; note = Oligonucleotide
cngcngcngcngcngcngcng

SEQ ID NO: 4-9 - skipped (6 entries)

SEQ ID NO: 10 - RNA, 30 nt, synthetic construct
  variation 1: note = c or m5c
  variation 2: note = u or m5u
  variation 4: note = c or m5c
  variation 5: note = u or m5u
  variation 7: note = c or m5c
  variation 8: note = u or m5u
  variation 10: note = c or m5c
  variation 11: note = u or m5u
  variation 13: note = c or m5c
  variation 14: note = u or m5u
  variation 16: note = c or m5c
  variation 17: note = u or m5u
  variation 19: note = c or m5c
  variation 20: note = u or m5u
  variation 22: note = c or m5c
  variation 23: note = u or m5u
  variation 25: note = c or m5c
  variation 26: note = u or m5u
  variation 28: note = c or m5c
  variation 29: note = u or m5u
  source 1..30: mol_type = other RNA; note = Oligonucleotide
nngnngnngnngnngnngnngnngnngnng

SEQ ID NO: 11 - RNA, 33 nt, synthetic construct
  variation 1: note = c or m5c
  variation 2: note = u or m5u
  variation 4: note = c or m5c
  variation 5: note = u or m5u
  variation 7: note = c or m5c
  variation 8: note = u or m5u
  variation 10: note = c or m5c
  variation 11: note = u or m5u
  variation 13: note = c or m5c
  variation 14: note = u or m5u
  variation 16: note = c or m5c
  variation 17: note = u or m5u
  variation 19: note = c or m5c
  variation 20: note = u or m5u
  variation 22: note = c or m5c
  variation 23: note = u or m5u
  variation 25: note = c or m5c
  variation 26: note = u or m5u
  variation 28: note = c or m5c
  variation 29: note = u or m5u
  variation 31: note = c or m5c
  variation 32: note = u or m5u
  source 1..33: mol_type = other RNA; note = Oligonucleotide
nngnngnngnngnngnngnngnngnngnngnng

SEQ ID NO: 12 - RNA, 36 nt, synthetic construct
  variation 1: note = c or m5c
  variation 2: note = u or m5u
  variation 4: note = c or m5c
  variation 5: note = u or m5u
  variation 7: note = c or m5c
  variation 8: note = u or m5u
  variation 10: note = c or m5c
  variation 11: note = u or m5u
  variation 13: note = c or m5c
  variation 14: note = u or m5u
  variation 16: note = c or m5c
  variation 17: note = u or m5u
  variation 19: note = c or m5c
  variation 20: note = u or m5u
  variation 22: note = c or m5c
  variation 23: note = u or m5u
  variation 25: note = c or m5c
  variation 26: note = u or m5u
  variation 28: note = c or m5c
  variation 29: note = u or m5u
  variation 31: note = c or m5c
  variation 32: note = u or m5u
  variation 34: note = c or m5c
  variation 35: note = u or m5u
  source 1..36: mol_type = other RNA; note = Oligonucleotide
nngnngnngnngnngnngnngnngnngnngnngnng

SEQ ID NO: 13-18 - skipped (6 entries)

SEQ ID NO: 19 - RNA, 30 nt, synthetic construct
  variation 1: note = c or m5c
  variation 3: note = c or m5c
  variation 4: note = c or m5c
  variation 6: note = c or m5c
  variation 7: note = c or m5c
  variation 9: note = c or m5c
  variation 10: note = c or m5c
  variation 12: note = c or m5c
  variation 13: note = c or m5c
  variation 15: note = c or m5c
  variation 16: note = c or m5c
  variation 18: note = c or m5c
  variation 19: note = c or m5c
  variation 21: note = c or m5c
  variation 22: note = c or m5c
  variation 24: note = c or m5c
  variation 25: note = c or m5c
  variation 27: note = c or m5c
  variation 28: note = c or m5c
  variation 30: note = c or m5c
  source 1..30: mol_type = other RNA; note = Oligonucleotide
ngnngnngnngnngnngnngnngnngnngn

SEQ ID NO: 20 - RNA, 33 nt, synthetic construct
  variation 1: note = c or m5c
  variation 3: note = c or m5c
  variation 4: note = c or m5c
  variation 6: note = c or m5c
  variation 7: note = c or m5c
  variation 9: note = c or m5c
  variation 10: note = c or m5c
  variation 12: note = c or m5c
  variation 13: note = c or m5c
  variation 15: note = c or m5c
  variation 16: note = c or m5c
  variation 18: note = c or m5c
  variation 19: note = c or m5c
  variation 21: note = c or m5c
  variation 22: note = c or m5c
  variation 24: note = c or m5c
  variation 25: note = c or m5c
  variation 27: note = c or m5c
  variation 28: note = c or m5c
  variation 30: note = c or m5c
  variation 31: note = c or m5c
  variation 33: note = c or m5c
  source 1..33: mol_type = other RNA; note = Oligonucleotide
ngnngnngnngnngnngnngnngnngnngnngn

SEQ ID NO: 21 - RNA, 36 nt, synthetic construct
  variation 1: note = c or m5c
  variation 3: note = c or m5c
  variation 4: note = c or m5c
  variation 6: note = c or m5c
  variation 7: note = c or m5c
  variation 9: note = c or m5c
  variation 10: note = c or m5c
  variation 12: note = c or m5c
  variation 13: note = c or m5c
  variation 15: note = c or m5c
  variation 16: note = c or m5c
  variation 18: note = c or m5c
  variation 19: note = c or m5c
  variation 21: note = c or m5c
  variation 22: note = c or m5c
  variation 24: note = c or m5c
  variation 25: note = c or m5c
  variation 27: note = c or m5c
  variation 28: note = c or m5c
  variation 30: note = c or m5c
  variation 31: note = c or m5c
  variation 33: note = c or m5c
  variation 34: note = c or m5c
  variation 36: note = c or m5c
  source 1..36: mol_type = other RNA; note = Oligonucleotide
ngnngnngnngnngnngnngnngnngnngnngnngn

SEQ ID NO: 22-27 - skipped (6 entries)

SEQ ID NO: 28 - RNA, 30 nt, synthetic construct; identical to SEQ ID NO: 10
  variation 1: note = c or m5c
  variation 2: note = c or m5c
  variation 4: note = c or m5c
  variation 5: note = c or m5c
  variation 7: note = c or m5c
  variation 8: note = c or m5c
  variation 10: note = c or m5c
  variation 11: note = c or m5c
  variation 13: note = c or m5c
  variation 14: note = c or m5c
  variation 16: note = c or m5c
  variation 17: note = c or m5c
  variation 19: note = c or m5c
  variation 20: note = c or m5c
  variation 22: note = c or m5c
  variation 23: note = c or m5c
  variation 25: note = c or m5c
  variation 26: note = c or m5c
  variation 28: note = c or m5c
  variation 29: note = c or m5c
  source 1..30: mol_type = other RNA; note = Oligonucleotide

SEQ ID NO: 29 - RNA, 33 nt, synthetic construct; identical to SEQ ID NO: 11
  variation 1: note = c or m5c
  variation 2: note = c or m5c
  variation 4: note = c or m5c
  variation 5: note = c or m5c
  variation 7: note = c or m5c
  variation 8: note = c or m5c
  variation 10: note = c or m5c
  variation 11: note = c or m5c
  variation 13: note = c or m5c
  variation 14: note = c or m5c
  variation 16: note = c or m5c
  variation 17: note = c or m5c
  variation 19: note = c or m5c
  variation 20: note = c or m5c
  variation 22: note = c or m5c
  variation 23: note = c or m5c
  variation 25: note = c or m5c
  variation 26: note = c or m5c
  variation 28: note = c or m5c
  variation 29: note = c or m5c
  variation 31: note = c or m5c
  variation 32: note = c or m5c
  source 1..33: mol_type = other RNA; note = Oligonucleotide

SEQ ID NO: 30 - RNA, 36 nt, synthetic construct; identical to SEQ ID NO: 12
  variation 1: note = c or m5c
  variation 2: note = c or m5c
  variation 4: note = c or m5c
  variation 5: note = c or m5c
  variation 7: note = c or m5c
  variation 8: note = c or m5c
  variation 10: note = c or m5c
  variation 11: note = c or m5c
  variation 13: note = c or m5c
  variation 14: note = c or m5c
  variation 16: note = c or m5c
  variation 17: note = c or m5c
  variation 19: note = c or m5c
  variation 20: note = c or m5c
  variation 22: note = c or m5c
  variation 23: note = c or m5c
  variation 25: note = c or m5c
  variation 26: note = c or m5c
  variation 28: note = c or m5c
  variation 29: note = c or m5c
  variation 31: note = c or m5c
  variation 32: note = c or m5c
  variation 34: note = c or m5c
  variation 35: note = c or m5c
  source 1..36: mol_type = other RNA; note = Oligonucleotide

SEQ ID NO: 31-40 - skipped (10 entries)

SEQ ID NO: 41 - RNA, 15 nt, synthetic construct
  variation 3: note = c or m5c
  variation 6: note = c or m5c
  variation 9: note = c or m5c
  variation 12: note = c or m5c
  variation 15: note = c or m5c
  source 1..15: mol_type = other RNA; note = Oligonucleotide
ggnggnggnggnggn

SEQ ID NO: 42 - RNA, 18 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  source 1..18: mol_type = other RNA; note = Oligonucleotide
ggnggnggnggnggnggn

SEQ ID NO: 43 - RNA, 21 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  source 1..21: mol_type = other RNA; note = Oligonucleotide
ggnggnggnggnggnggnggn

SEQ ID NO: 44 - RNA, 24 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  variation 24: note = c or m5c
  source 1..24: mol_type = other RNA; note = Oligonucleotide
ggnggnggnggnggnggnggnggn

SEQ ID NO: 45 - RNA, 27 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  variation 24: note = c or m5c
  variation 27: note = c or m5c
  source 1..27: mol_type = other RNA; note = Oligonucleotide
ggnggnggnggnggnggnggnggnggn

SEQ ID NO: 46 - RNA, 30 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  variation 24: note = c or m5c
  variation 27: note = c or m5c
  variation 30: note = c or m5c
  source 1..30: mol_type = other RNA; note = Oligonucleotide
ggnggnggnggnggnggnggnggnggnggn

SEQ ID NO: 47 - RNA, 33 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  variation 24: note = c or m5c
  variation 27: note = c or m5c
  variation 30: note = c or m5c
  variation 33: note = c or m5c
  source 1..33: mol_type = other RNA; note = Oligonucleotide
ggnggnggnggnggnggnggnggnggnggnggn

SEQ ID NO: 48 - RNA, 36 nt, synthetic construct
  variation 3: note = c or m5c
  variation 6: note = c or m5c
  variation 9: note = c or m5c
  variation 12: note = c or m5c
  variation 15: note = c or m5c
  variation 18: note = c or m5c
  variation 21: note = c or m5c
  variation 24: note = c or m5c
  variation 27: note = c or m5c
  variation 30: note = c or m5c
  variation 33: note = c or m5c
  variation 36: note = c or m5c
  source 1..36: mol_type = other RNA; note = Oligonucleotide
ggnggnggnggnggnggnggnggnggnggnggnggn

SEQ ID NO: 49 - skipped

SEQ ID NO: 50 - RNA, 15 nt, synthetic construct
  variation 1: note = c or m5c
  variation 4: note = c or m5c
  variation 7: note = c or m5c
  variation 10: note = c or m5c
  variation 13: note = c or m5c
  source 1..15: mol_type = other RNA; note = Oligonucleotide
nggnggnggnggngg

SEQ ID NO: 51 - RNA, 18 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  source 1..18: mol_type = other RNA; note = Oligonucleotide
nggnggnggnggnggngg

SEQ ID NO: 52 - RNA, 21 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  source 1..21: mol_type = other RNA; note = Oligonucleotide
nggnggnggnggnggnggngg

SEQ ID NO: 53 - RNA, 24 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  variation 22: note = c or m5c
  source 1..24: mol_type = other RNA; note = Oligonucleotide
nggnggnggnggnggnggnggngg

SEQ ID NO: 54 - RNA, 27 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  variation 22: note = c or m5c
  variation 25: note = c or m5c
  source 1..27: mol_type = other RNA; note = Oligonucleotide
nggnggnggnggnggnggnggnggngg

SEQ ID NO: 55 - RNA, 30 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  variation 22: note = c or m5c
  variation 25: note = c or m5c
  variation 28: note = c or m5c
  source 1..30: mol_type = other RNA; note = Oligonucleotide
nggnggnggnggnggnggnggnggnggngg

SEQ ID NO: 56 - RNA, 33 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  variation 22: note = c or m5c
  variation 25: note = c or m5c
  variation 28: note = c or m5c
  variation 31: note = c or m5c
  source 1..33: mol_type = other RNA; note = Oligonucleotide
nggnggnggnggnggnggnggnggnggnggngg

SEQ ID NO: 57 - RNA, 36 nt, synthetic construct
  variation 1: note = c or m5c
  variation 4: note = c or m5c
  variation 7: note = c or m5c
  variation 10: note = c or m5c
  variation 13: note = c or m5c
  variation 16: note = c or m5c
  variation 19: note = c or m5c
  variation 22: note = c or m5c
  variation 25: note = c or m5c
  variation 28: note = c or m5c
  variation 31: note = c or m5c
  variation 34: note = c or m5c
  source 1..36: mol_type = other RNA; note = Oligonucleotide
nggnggnggnggnggnggnggnggnggnggnggngg

SEQ ID NO: 58-64 - skipped (7 entries)

SEQ ID NO: 65 - RNA, 20 nt, synthetic construct
  variation 1: note = c or m5c
  variation 2: note = a or 2,6-diaminopurine
  variation 5: note = c or m5c
  variation 6: note = a or 2,6-diaminopurine
  variation 9: note = c or m5c
  variation 10: note = a or 2,6-diaminopurine
  variation 13: note = c or m5c
  variation 14: note = a or 2,6-diaminopurine
  variation 17: note = c or m5c
  variation 18: note = a or 2,6-diaminopurine
  source 1..20: mol_type = other RNA; note = Oligonucleotide
nnggnnggnnggnnggnngg

SEQ ID NO: 66 - RNA, 24 nt, synthetic construct
  variation 1: note = c or m5c
  variation 2: note = a or 2,6-diaminopurine
  variation 5: note = c or m5c
  variation 6: note = a or 2,6-diaminopurine
  variation 9: note = c or m5c
  variation 10: note = a or 2,6-diaminopurine
  variation 13: note = c or m5c
  variation 14: note = a or 2,6-diaminopurine
  variation 17: note = c or m5c
  variation 18: note = a or 2,6-diaminopurine
  variation 21: note = c or m5c
  variation 22: note = a or 2,6-diaminopurine
  source 1..24: mol_type = other RNA; note = Oligonucleotide
nnggnnggnnggnnggnnggnngg

SEQ ID NO: 67 - RNA, 28 nt, synthetic construct
  variation 1: note = c or m5c
  variation 2: note = a or 2,6-diaminopurine
  variation 5: note = c or m5c
  variation 6: note = a or 2,6-diaminopurine
  variation 9: note = c or m5c
  variation 10: note = a or 2,6-diaminopurine
  variation 13: note = c or m5c
  variation 14: note = a or 2,6-diaminopurine
  variation 17: note = c or m5c
  variation 18: note = a or 2,6-diaminopurine
  variation 21: note = c or m5c
  variation 22: note = a or 2,6-diaminopurine
  variation 25: note = c or m5c
  variation 26: note = a or 2,6-diaminopurine
  source 1..28: mol_type = other RNA; note = Oligonucleotide
nnggnnggnnggnnggnnggnnggnngg

SEQ ID NO: 68 - RNA, 32 nt, synthetic construct
  variation 1: note = c or m5c
  variation 2: note = a or 2,6-diaminopurine
  variation 5: note = c or m5c
  variation 6: note = a or 2,6-diaminopurine
  variation 9: note = c or m5c
  variation 10: note = a or 2,6-diaminopurine
  variation 13: note = c or m5c
  variation 14: note = a or 2,6-diaminopurine
  variation 17: note = c or m5c
  variation 18: note = a or 2,6-diaminopurine
  variation 21: note = c or m5c
  variation 22: note = a or 2,6-diaminopurine
  variation 25: note = c or m5c
  variation 26: note = a or 2,6-diaminopurine
  variation 29: note = c or m5c
  variation 30: note = a or 2,6-diaminopurine
  source 1..32: mol_type = other RNA; note = Oligonucleotide
nnggnnggnnggnnggnnggnnggnnggnngg

SEQ ID NO: 69 - RNA, 36 nt, synthetic construct
  variation 1: note = c or m5c
  variation 2: note = a or 2,6-diaminopurine
  variation 5: note = c or m5c
  variation 6: note = a or 2,6-diaminopurine
  variation 9: note = c or m5c
  variation 10: note = a or 2,6-diaminopurine
  variation 13: note = c or m5c
  variation 14: note = a or 2,6-diaminopurine
  variation 17: note = c or m5c
  variation 18: note = a or 2,6-diaminopurine
  variation 21: note = c or m5c
  variation 22: note = a or 2,6-diaminopurine
  variation 25: note = c or m5c
  variation 26: note = a or 2,6-diaminopurine
  variation 29: note = c or m5c
  variation 30: note = a or 2,6-diaminopurine
  variation 33: note = c or m5c
  variation 34: note = a or 2,6-diaminopurine
  source 1..36: mol_type = other RNA; note = Oligonucleotide
nnggnnggnnggnnggnnggnnggnnggnnggnngg

SEQ ID NO: 70 - DNA, 23 bp, synthetic construct
  misc_feature 1..23: note = Primer
  source 1..23: mol_type = other DNA
atggcgaccctggaaaagctgat

SEQ ID NO: 71 - DNA, 17 bp, synthetic construct
  misc_feature 1..17: note = Primer
  source 1..17: mol_type = other DNA
tgaggcagcagcggctg

SEQ ID NO: 72 - DNA, 20 bp, synthetic construct
  misc_feature 1..20: note = Primer
  source 1..20: mol_type = other DNA
ggacttcgagcaagagatgg

SEQ ID NO: 73 - DNA, 20 bp, synthetic construct
  misc_feature 1..20: note = Primer
  source 1..20: mol_type = other DNA
agcactgtgttggcgtacag

SEQ ID NO: 74 - DNA, 18 bp, synthetic construct
  misc_feature 1..18: note = Primer
  source 1..18: mol_type = other DNA
cgccgcctcctcagcttc

SEQ ID NO: 75 - DNA, 24 bp, synthetic construct
  misc_feature 1..24: note = Primer
  source 1..24: mol_type = other DNA
gagagttccttctttggtcggtgc

SEQ ID NO: 76 - DNA, 23 bp, synthetic construct
  misc_feature 1..23: note = Primer
  source 1..23: mol_type = other DNA
tgggaaacagataaaactccaga

SEQ ID NO: 77 - DNA, 26 bp, synthetic construct
  misc_feature 1..26: note = Primer
  source 1..26: mol_type = other DNA
aatatgaccttgtgatagaacgaaag

SEQ ID NO: 78 - DNA, 20 bp, synthetic construct
  misc_feature 1..20: note = Primer
  source 1..20: mol_type = other DNA
aaatgagctggtgcagaagg

SEQ ID NO: 79 - DNA, 19 bp, synthetic construct
  misc_feature 1..19: note = Primer
  source 1..19: mol_type = other DNA
ggctgccatgtcatcgtat

SEQ ID NO: 80 - DNA, 13481 bp, Homo sapiens
  source 1..13481: mol_type = other DNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 81 - DNA, 10636 bp, Homo sapiens
  source 1..10636: mol_type = other DNA
gaggagagagcagagtataccgcagacatcatttctactacagtggcggagccgtacaggacctgtttcactgcagggggatccaaaacaagccccgtggagcagcagccagagcaacagcagccgcaagacattgtttctctccctctgcccccccttccccacgcaaccccagatccatttacactttacagttttacctcacaaaaactactacaagcaccaagctccctgatggaaaggagcatcgtgcatcaagtcaccagggtggtccattcaagctgcagatttgtttgtcatccttgtacagcaatctcctcctccactgccactacagggaagtgcatcacatgtcagcatactggagcatagtgaaagagtctattttgaagcttcaaacttagtgctgctgcagaccaggaacaagagagaaagagtggatttcagcctgcacggatggtcttgaaacacaaatggtttttggtctaggcgttttacactgagattctccactgccaccctttctactcaagcaaaatcttcgtgaaaagatctgctgcaaggaactgatagcttatggttctccattgtgatgaaagcacatggtacagttttccaaagaaattagaccattttcttcgtgagaaagaaatcgacgtgctgttttcatagggtatttctcacttctctgtgaaaggaagaaagaacacgcctgagcccaagagccctcaggagccctccagagcctgtgggaagtctccatggtgaagtataggctgaggctacctgtgaacagtacgcagtgaatgttcatccagagctgctgttggcggattgtacccacggggagatgattcctcatgaagagcctggatcccctacagaaatcaaatgtgactttccgtttatcagactaaaatcagagccatccagacagtgaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtggctctcccttatcatttgtatccagattactgtactgtaggctaaaataacacagtatttacatgttatcttcttaattttaggtttctgttctaaccttgtcattagagttacagcaggtgtgtcgcaggagactggtgcatatgctttttccacgagtgtctgtcagtgagcgggcgggaggaagggcacagcaggagcggtcagggctccaggcatccccggggaagaaaggaacggggcttcacagtgcctgccttctctagcggcacagaagcagccgggggcgctgactcccgctagtgtcaggagaaaagtcccgtgggaagggtcctgcaggggtgcagggttgcacgcatgtgggggtgcacaggcgctgtggcggcgagtgagggtctctttttctctgcctccctctgcctcactctcttgctatcggcatgggccgggggggttcagagcagtgtcctcctggggttcccacgtgcaaaatcaacatcaggaacccagcttcagggcatcgcggagacgcgtcagatggcagatttggaaagttaaccatttaaaagaacatttttctctccaacatattttacaataaaagcaacttttaattgtatagatatatatttccccctatggggcctgactgcactgatatatattttttttaaagagcaactgccacatgcgggatttcatttctgctttttactagtgcagcgatgtcaccagggtgttgtggtggacagggaagcccctgctgtcatggccccacatggggtaaggggggttgggggtgggggagagggagagagcgaacacccacgctggtttctgtgcagtgttaggaaaaccaatcaggttattgcattgacttcactcccaagaggtagatgcaaactgcccttcagtgagagcaacagaagctcttcacgttgagtttgcgaaatctttttgtctttgaactctagtactgtttatagttcatgactatggacaactcgggtgccactttttttttttttcagattccagtgtgacatgaggaattagattttgaagatgagcatatattactatctttaagcatttaaaaatactgttcacactttattaccaagcatcttggtctctcattcaacaagtactgtatctcactttaaactctttggggaaaaaacaaaaacaaaaaaaactaagttgctttctttttttcaacactgtaactacatttcagctctgcagaattgctgaagagcaagatattgaaagtttcaatgtggtttaaagggatgaatgtgaattatgaactagtatgtgacaataaatgaccaccaagtactacctgacgggaggcacttttcactttgatgtctgagaatcagttcaaggcatatgcagagttggcagagaaactgagagaaaagggatggagaagagaatactcatttttgtccagtgtttttctttttaagatgaacttttaaagaaccttgcgatttgcacatattgagtttataacttgtgtgatattcctgcagtttttatccaataacattgtgggaaaggtttgggggactgaacgagcataaataaatgtagcaaaatttctttctaacctgcctaaactctaggccattttataaggttatgttcctttgaaaattcattttggtctttttaccacatctgtcacaaaaagccaggtcttagcgggctcttagaaactctgagaattttcttcagattcattgagagagttttccataaagacatttatatatgtgagcaagattttttttaaacaattactttattattgttgttattaatgttattttcagaatggcttttttttttctattcaaaatcaaatcgagatttaatgtttggtacaaacccagaaagggtatttcatagtttttaaacctttcattcccagagatccgaaatatcatttgtgggttttgaatgcatctttaaagtgctttaaaaaaaagttttataagtagggagaaatttttaaatattcttacttggatggctgcaactaaactgaacaaatacctgacttttcttttaccccattgaaaatagtactttcttcgtttcacaaattaaaaaaaaaatctggtatcaacccacattttggctgtctagtattcatttacatttagggttcaccaggactaatgatttttataaaccgttttctggggtgtaccaaaaacatttgaataggtttagaatagctagaatagttccttgactttcctcgaatttcattaccctctcagcatgcttgcagagagctgggtgggctcattcttgcagtcatactgcttatttagtgctgtattttttaaacgtttctgttcagagaacttgcttaatcttccatatattctgctcagggcacttgcaattattaggttttgtttttctttttgttttttagcctttgatggtaagaggaatacgggctgccacatagactttgttctcattaatatcactatttacaactcatgtggactcagaaaaacacacaccaccttttggcttacttcgagtattgaattgactggatccactaaaccaacactaagatgggaaaacacacatggtttggagcaataggaacatcatcataatttttgtggttctatttcaggtataggaattataaaataattggttctttctaaacacttgtcccatttcattctcttgcttttttagcatgtgcaatactttctgtgccaatagagtctgaccagtgtgctatatagttaaagctcattcccttttggctttttccttgtttggttgatcttccccattctggccagagcagggctggagggaaggagccaggagggagagagcctcccacctttcccctgctgcggatgctgagtgctggggcggggagccttcaggagccccgtgcgtctgccgccacgttgcagaaagagccagccaaggagacccgggggaggaaccgcagtgtcccctgtcaccacacggaatagtgaatgtggagtgtggagaggaaggaggcagattcatttctaagacgcactctggagccatgtagcctggagtcaacccattttccacggtcttttctgcaagtgggcaggcccctcctcggggtctgtgtccttgagacttggagccctgcctctgagcctggacgggaagtgtggcctgttgtgtgtgtgcgttctgagcgtgttggccagtggctgtggaggggaccacctgccacccacggtcaccactcccttgtggcagctttctcttcaaataggaagaacgcacagagggcaggagcctcctgtttgcagacgttggcgggccccgaggctcccagagcagcctctgtcaccgcttctgtgtagcaaacattaacgatgacaggggtagaaattcttcggtgccgttcagcttacaaggatcagccatgtgcctctgtactatgtccactttgcaatatttaccgacagccgtcttttgttctttctttcctgttttccatttttaaactagtaacagcaggccttttgcgtttacaatggaacacaatcaccaagaaattagtcagggcgaaaagaaaaaaataatactattaataagaaaccaacaaacaagaacctctctttctagggatttctaaatatataaaatgactgttccttagaatgtttaacttaagaattatttcagtttgtctgggccacactggggcagaggggggagggagggatacagagatggatgccacttacctcagatcttttaaagtggaaatccaaattgaattttcatttggactttcaggataattttctatgttggtcaacttttcgttttccctaactcacccagtttagtttgggatgatttgatttctgttgttgttgatcccatttctaacttggaattgtgagcctctatgttttctgttaggtgagtgtgttgggttttttccccccaccaggaagtggcagcatccctccttctcccctaaagggactctgcggaacctttcacacctctttctcagggacggggcaggtgtgtgtgtggtacactgacgtgtccagaagcagcactttgactgctctggagtagggttgtacaatttcaaggaatgtttggatttcctgcatcttgtggattactccttagataccgcatagattgcaatataatgctgcatgttcaagatgaacagtagctcctagtaatcataaaatccactctttgcacagtttgatctttactgaaatatgttgccaaaatttatttttgttgttgtagctctggattttgttttgttttgttttttaaggaaacgattgacaataccctttaacatctgtgactactaaggaaacctatttctttcatagagagaaaaatctccaatgcttttgaagacactaataccgtgctatttcagatatgggtgaggaagcagagctctcggtaccgaaggccgggcttcttgagctgtgttggttgtcatggctactgtttcatgaaccacaagcagctcaacagactggtctgttgccttctgaaaccctttgcacttcaatttgcaccaggtgaaaacagggccagcagactccatggcccaattcggtttcttcggtggtgatgtgaaaggagagaattacactttttttttttttaagtggcgtggaggcctttgcttccacatttgtttttaacccagaatttctgaaatagagaatttaagaacacatcaagtaataaatatacagagaatatacttttttataaagcacatgcatctgctattgtgttgggttggtttcctctcttttccacggacagtgttgtgtttctggcatagggaaactccaaacaacttgcacacctctactccggagctgagatttcttttacatagatgacctcgcttcaaatacgttaccttactgatgataggatcttttcttgtagcactataccttgtgggaatttttttttaaatgtacacctgatttgagaagctgaagaaaacaaaattttgaagcactcactttgaggagtacaggtaatgttttaaaaaattgcacaaaagaaaaatgaatgtcgaaatgattcattcagtgtttgaaagatatggctctgttgaaacaatgagtttcatactttgtttgtaaaaaaaaaaaagcagagaagggttgaaagttacatgtttttttgtatatagaaatttgtcatgtctaaatgatcagatttgtatggttatggcctggaagaattactacgtaaaaggctcttaaactatacctatgcttattgttatttttgttacatatagccctcgtctgagggaggggaactcggtattctgcgatttgagaatactgttcattcctatgctgaaagtacttctctgagctcccttcttagtctaaactcttaagccattgcaacttctttttcttcagagatgatgtttgacattttcagcacttcctgttcctataaacccaaagaatataatcttgaacacgaagtgtttgtaacaagggatccaggctaccaatcaaacaggactcattatggggacaaaaaaaaaaattatttcaccttctttccccccacacctcatttaaatggggggagtaaaaacatgatttcaatgtaaatgcctcattttattttagttttattttgatttttatttaatataaagaggccagaataaatacggagcatcttctcagaatagtattcctgtccaaaaatcaagccggacagtggaaactggacagctgtggggatattaagcacccccacttacaattcttaaattcagaatctcgtcccctcccttctcgttgaaggcaactgttctggtagctaactttctcctgtgtaatggcgggagggaacaccggcttcagtttttcatgtccccatgacttgcatacaaatggttcaactgtattaaaattaagtgcatttggccaataggtagtatctatacaataacaacaatctctaagaatttccataacttttcttatctgaaaggactcaagtcttccactgcagatacattggaggcttcacccacgttttctttccctttagtttgtttgctgtctggatggccaatgagcctgtctccttttctgtggccaatctgaaggccttcgttggaagtgttgtttacagtaatccttaccaagataacatactgtcctccagaataccaagtattaggtgacactagctcaagctgttgtcttcagagcagttaccaagaagctcggtgcacaggttttctctggttcttacaggaaccacctactctttcagttttctggcccaggagtggggtaaatcctttagttagtgcatttgaacttgatacctgtgcattcagttctgtgaatactgccctttttggcggggtttcctcatctccccagcctgaactgctcaactctaaacccaaattagtgtcagccgaaaggaggtttcaagatagtcctgtcagtatttgtggtgaccttcagattagacagtcttcatttccagccagtggagtcctggctccagagccatctctgagactcgtactactggatgttttaatatcagatcattacccaccatatgcctcccacaggccaagggaaaacagacaccagaacttgggttgagggcactaccagactgacatggccagtacagaggagaactagggaaggaatgatgttttgcaccttattgaaaagaaaattttaagtgcatacataatagttaagagcttttattgtgacaggagaacttttttccatatgcgtgcatactctctgtaattccagtgtaaaatattgtacttgcactagcttttttaaacaaatattaaaaaatggaagaattcatattctattttctaatcgtggtgtgtctatttgtaggatacactcgagtctgtttattgaattttatggtccctttctttgatggtgcttgcaggttttctaggtagaaattatttcattattataataaaacaatgtttgattcaaaatttgaacaaaattgttttaaataaattgtctgtataccagtacaagtttattgtttcagtatactcgtactaataaaataacagtgccaattgca

SEQ ID NO: 82 - DNA, 4712 bp, Homo sapiens
  source 1..4712: mol_type = other DNA
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 83 - DNA, 6923 bp, Homo sapiens
  source 1..6923: mol_type = other DNA
gagaggggcagggggcggagctggagggggtggttcggcgtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 84 - DNA, 8646 bp, Homo sapiens
  source 1..8646: mol_type = other DNA
gatgtcccgagctgctatccccggctcggcccgggcagccgccttctgagcccccgacccgaggcgccgagccgccgccgcccgatgggctgggccgtggagcgtctccgcagtcgtagctccagccgccgcgctcccagccccggcagcctcagcatcagcggcggcggcggcggcggcggcgtcttccgcatcgttcgccgcagcgtaacccggagccctttgctctttgcagaatggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggagtggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggtggaggcggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggagggaccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaatccgaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttggggccgcatgccttacctggacatgtatcagatgctgagacacatgtctccgcccctgggtctggggaagaagtgtccggccagagtggcttacaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcagcagcaggcggtggccaggccgggccgggcggccaccagcggccctcggaggtacccaggccccacggccgagcctctggccggagatcggccgcccacggggggccacagcagcggccgctcgcccaggatggagaggcgggtcccaggcccggcccggagcgagtcccccagggcctgtcgacacggcggggcccggtggccggcatctggcccgcacgtgtccgaggggcccccgggtccccggcaccatggctactaccggggctccgactacgacgaggccgatggcccgggcagcgggggcggcgaggaggccatggccggggcctacgacgcgccaccccccgtacgacacgcgtcctcgggcgccaccgggcgctcgcccaggactccccgggcctcgggcccggcctgcgcctcgccttctcggcacggccggcgactccccaacggctactacccggcgcacggactggccaggccccgcgggccgggctccaggaagggcctgcacgaaccctacagcgagagtgacgatgattggtgctaagcccgggcgaggtggcgcccgcccggccccccacgcaccccacgcacacaccccacccgaggagccgcgcagaggccgcgggggcccagcacagagggcccgggagagggccagccgggagaccccagactctggagaggccagggctgggccacaagggtgtcccgcagagaccctcggccaaaagagaccctcctgggcagccacggcgccccccaaccagccccgatccccccacccacgacaggggctctcgggtgggaggcagggagcagacaaaccacacagccaagggatttgaattaactcagccatttttggagaactttggggaacatgaaaaaaaaaaaaaaaaaaaaaaaaaaaaacatttttaaaagaaaaaacggggagaaaaaaatagcttctattgatgagttttatcatctcaattgaatctttcctttccctgatgaagacagctggtggccgagtgcggcaaagaagccagaaggaaccagaatcccagtgccctacacccaccaccagacacactcacacccacacacgttctcagacacacacaagagtgcttgccggttataccaaaccctactattactgcctgcagaaatcaatttaaaaaaataataataacaataaacaattttaaaaaggacaaaaaaattaatgattgagaaaagaggcatttttttctgacatttggtcctgcttgaaacaacaaaagaagaagaaaaacccaccatcaccaccgattcctttgcttcttttttccttttttcctaccttgtttgaaaaccgtgggcttgggactgtgaattattgcatgacattcaaaaagaaaaaaaaaataaaaaaaagttgaatcaaa

SEQ ID NO: 85 - DNA, 7242 bp, Homo sapiens
  source 1..7242: mol_type = other DNA
gaaaaagggtgaaagagaaacttggcgacctcccggaggagttcgcgaagcgaccaggagcgtgttgccatcgtcctcacccggcacccaattccaccacagagtcgggatttcgtcggtgatcgtgatggggtgcttttatttttctctttgattttcaaaaaatgtctatgtgactgtccctatcttaaggggaagttgaaagtgggggcgggggtgctcaatgagaaacgttgccttgtgtgtagttgtttggagcacactgcaaattatattggcatctctttccaaaagtcactttgattcaacttcgatagctttctcgtaaatggcacgtttaggtggtgagaggtggatgaggaaacaggcaccagtgcagctgatttgacctccagtgggatagatacgattagcaccaggatcgtgtctcattttgaacccagatctgaacagaattaagacgaacgagctttcacaattgcagcagatgaagatccattggtaaattgatcaggatttttggcctaccctccaaagaaaaggagcggaaagaatgtcggagcgggccgcggatgacgtcaggggggagccgcgccgcgcggcggcggcggcgggcggagcagcggccgcggccgcccggcagcagcagcagcagcagcagcagcagcagccgccgcctccgcagccccagcggcagcagcacccgccaccgccgccacggcgcacacggccggaggacggcgggcccggcgccgcctccacctcggccgccgcaatggcgacggtcggggagcgcaggcctctgcccagtcctgaagtgatgctgggacagtcgtggaatctgtgggttgaggcttccaaacttcctgggaaggacgggacagaattggacgaaagtttcaaggagtttgggaaaaaccgcgaagtcatggggctctgtcgggaagacatgccaatatttggtttctgtccagcccatgatgatttctacttggtggtgtgtaacgactgtaatcaggttgtcaaaccgcaggcatttcaatcacattatgaaagaagacatagctcatccagcaagccgcctttggccgttcctcccacttcagtattttccttcttcccttctctgtccaaaagcaaaggaggcagtgcaagtggaagcaaccgttcttccagtggaggtgttcttagcgcatcctcatcaagttccaagttgttgaaatcacccaaagagaaactgcagctcagggggaacaccaggccaatgcatcccattcagcaaagtagagttccccatggtagaatcatgacaccctctgtgaaagtggaaaagattcatccgaaaatggatggcacactactgaaatctgcggtggggccaacctgtcctgctactgtgagttccttagtcaagcctggccttaactgcccctcaataccaaagccaaccttgccttcacctggacagattctgaatggcaaagggcttcctgcaccgcccactctggaaaagaaacctgaagacaattccaataataggaaatttttaaataagagattatcagaaagagagtttgatcctgacatccactgtggggttattgatctcgacaccaagaagccctgcacccggtctttgacatgcaagacacattccttaacccagcgcagggctgtccagggtagaagaaaacgatttgatgtgttattagccgagcacaaaaacaaaaccagggaaaaggaattgattcgccatccggactctcagcaaccaccgcagcctctcagggacccgcatcccgcccctcctagaacgtcacaggagccgcaccaaaaccctcacggagtgattccttccgaatcaaagccttttgtagctagtaaacctaaacctcacacccccagtcttccaaggcctccaggctgccctgctcagcaaggtgggagtgcccccattgaccctcctccagtccatgaatctccacaccctcccctgcctgccactgagccagcttctcggttatccagtgaggagggcgaaggcgatgacaaagaagagtctgttgaaaaactggactgtcattattcaggtcatcatcctcagccagcatctttttgcacatttgggagccggcagataggaagaggctattacgtgtttgactccaggtggaatcgacttcgctgcgccctcaacctcatggtggagaagcatctgaatgcacagctatggaagaaaatcccaccagtgcccagtaccacctcacccatctccacacgtattcctcaccggacaaactctgtgccgacatcacaatgtggagtcagctatctggcagcagccaccgtctctacatccccagtcctgctctcatctacctgcatctccccaaatagcaaatcggtaccagctcatggaaccacactaaatgcacagcctgctgcttcaggggcgatggatcctgtgtgcagtatgcaatccagacaagtgtcctcttcatcctcatccccttccacgccctctggcctttcctcggttccttcctcccccatgtccaggaaacctcagaaattgaaatccagcaaatctttgaggcccaaggagtcttctggtaacagcactaactgtcaaaatgccagtagcagtaccagtggcggctcaggaaagaaacgcaaaaacagttccccactgttggttcactcttcctcctcctcttcctcctcctcctcttcttctcattccatggagtcttttaggaaaaactgtgtggctcactctgggcctccctacccctcaacggtaacatcttcccatagcatcggcctcaactgtgtgacgaataaagcaaatgcggtgaacgtccggcatgaccagtcagggaggggcccccccaccgggagccctgctgaatccatcaagaggatgagtgtgatggtgaacagcagtgattctactctttctcttgggccattcattcaccagtccaatgaactgcctgtcaactcccacggcagtttttcccactcacacactcctctagacaaactcataggaaagaaaagaaagtgctcacccagctcgagcagcatcaacaacagcagcagcaaacccacaaaggttgccaaagtgccagccgtgaacaatgtccacatgaaacacacaggcaccatcccaggggcacaaggactgatgaacagttccctccttcatcagccaaaggcacgtccctgacagctgaaaatagcacggggaggaataatgcggacacttttgaggacaagttacacctccactcagcactctggactccacgatgcctttgagtctgttttcccaacctcctgtgggcctcaagggtagaaacctgccgggctgttgttttaacgaggatttccctgaagctatgtctctagcagtgagtactcataaaggacactggatcaagttcagccaccgaattgcttttatcagtgttaaagtggtctgaactgcttgctaccaatctgtgagaagtttttgtttttgttttgttttttaacttgcagtatatcacagagccactcttcaagtagattggctgggcaaaagaatgttttggcaagagcgttactgtagacctttctccctccttccttttactaccatttttttttaacactgtcatctgtaggtcactctccagcagttaggcaccttaactggagaccagaaaccttccagagaacacagggctgcatcccgagcaaccctctgaagaagggaattaggctttagattttgatagcaatgttccaggaatgaaatatagatgttagcccaagacaccatgacaaaatagcccagccttttgagagtaatttgggaaaagaagctgtcagaagtttctaacttacaaactggtttgaaatttttgatgcccagacagcaagtataaatcattttggaggcttacttttcatgatacaaaagcaattctgtgtgatttttttttttaagaagaaagaaaatgcaagctagttttgagaaaggaaggccaaattgggtcgggggagggtgggagtgaggaagttaaaatcactatagggagaaaaaacttttttcaagatttccaaagagatgaaattttcttaatccttttaagttttcatagtaaacagtatggcagattgggttggttgtcctacctggtctatttttaaaagtcaccttttaaagtgacattattagatacacttaaatgtttccaaggcactctctacattacccttgtttttctctttggatactgtcctgggactaagtgtagatttctgcttcaagcacttctggcattgtgtgtttttgtatgcactccccttcatgccacttcagatgtttatttggatgtggttggggacgagagcagacaccaaggaaagggagttggagagaatgtaagtcctgacctgaaggtcttttgtgatgcatgtataggattgccctgacacacaccctcctttcttgggattataccagccatcttcctgagagttttggagccctctaggatattttttctagtaccccaccccccacccctaaagaaagaccttaatatgttaaaacagcattgcttggagaaggtgtcattgaattccgggacgagccggagcctttaaatgggtgcttccaccactacaggctcctgacacgagtaacaggcactgttgcttagaagaacacacgaagttgccgaacacagggtaaaatttccaaggcgctgatcgttgccctggccagggcctgatgagagccagtcagtacattctttttttcctacagttcttgggtttcaaacttcagtttcagggaatttcaagtcaacaacaggtagaatgaataaacttggttaccagcctaataatgtgaattgctacagaattattcttattatgtaagaaaacaaaaactttatgcagatactttagctataaattgatgtaaaatactgatttttttaaaggaaggagagaacagtatcttgttcaattattatgcaatcaatcagtaaatgtttttaaaatgatactacaggagagcttagtaaggagagggcatggatgggccagtttggcatagttgggagaaatcagtctggtttccatcccagtcggggaagagagaggtgagagggaatcagaacgtacctagttgattccttggtgacaagtgcaatggggtatgggtagaatttattttcagagccaagaggacttgatggttataaataaagttgcctttagcaatggaatttacagatcgatcatgttgttccgaaagatgtgaataggatccacaataacaagttgattcagactaatgtagatatttagattagcaagtattgaacatttgatttcttagactgaggttttaaatgaatttcattatttctcccggtaatacacagagcatcgggattaggaaattagccattttggattctgtctgccacaaacagacctattctaaacagtgctattaaattttagactgttgttcaaatattttattttctcctaaactactttttatggtgatgaataattaagaccatttaaaacacgggagtacaagtatttttttgaattaaattaacttgcaaaaaccaaatttgcagtggttgggttgtttctagacagactttggtatcaatttaaaaccaagataatttttatattctttccaatagaatttcatgacaactcctgcagttttcttaacctacaaaaaaaaaaaaaaagtgctgcaatgatgaacaaagaattatacaaaacctacttttgtatctttattttggaatttcttgttctattataaatatggaagtatccctatttcagaagacatattttgttaaaaatgaattgtacatatttaaatatgtatttttgcacaggtctttttttctgatatcctgttttggtacaattgagatcatctagtatttatttattaattaataaaagtaaatacctttttataatttgaagtggttcactgccaagccaatagttctagaacctgcctcctttacagttttaagggatgcctctgttctgtgaaaagtctttgtcccttttagtgtctcgggagtggattcatacagatgaagtgggcattgcttcttcctggttgcctggtttcccgatagactacatgtaactaagtgacacactgcttttcttttgttagtattttatgtgtgggagtatgtgactgcgtgtgtgtgtgcctgtgcgtgtgtgtgtatactcagcacatgtatgttactatgtgatgtggtttaaaactaatggaaaaaactgaaagtgcctgaaactagttttaagcttagacagaatctctttaaaaagacttgaatgttcagttgaacttttggagtttgctttttccacctaaatattgtttctaaaattttaggggcggagttgaaaaccaccaatgctggtaattttataaggtattttaaaattaagacctcttggttcatagtaattcaattggtgatggattgcctggtggcaccaagggttacagatctttttgtcagccagcaacttacacgatgtgtccgttttgttattcactcatgaaatacaatttaaaatatttaaaatattttgtattccaaaaatataatacaaagaagtacctctgagaacattgaaaaaaatgtataatttgaaaaatgtaacttataagggcagctgtcttcctgcaagagttctgttgccaaggaatttcttaatgtcgctcattctgtccagggggtttggggcgattgggctttgaaaaaatattcttaaaagtttgtttagcagaaaataatcacttttacattttgtgcaaaacattttacattttcagataatttaaatgagcactacatactgtcagtgtgaatgtagggccttgaaagcattttgcttttttttttttttttttttttttgagcttggttataaaagcaatctcctgtgttaaaaacgtgtgaatagtttgataatttgtacacataatcaagagcatctcagctggactttgtgttggctgctgtatagaacatgaacaaatgtcaagggatagaaaattactttggatatttaaaagagaagaaatatatagtctatttgttttgtaacaagtttctgtaaataaaataatttatactatttataagaaaaaaaaaaaaaaaaaaa

SEQ ID NO: 86 - DNA, 2300 bp, Homo sapiens
  source 1..2300: mol_type = other DNA
ggccaggcaagcctgaatcctgtccctgccatctcgccactgcagctcgggtccagaaaggcaccattttgtcgcggctgcccgctctcccagggggaggagggatcttttttgcattttggagcggctgccaaggaggggaacctgttgggcatctccccagacccgcttgtgagcgcctccggggcgggcgggcgggaccagacccctcggggcacggcgtatcttggcacccggaggcagcggaggcaggcgcagcatcctcgctgggaactggagctggagtgagcgcaccgcgcgggaggagccgccgcagcctcgcagaacccgagtggaggaggtgacagctccattgccgggtttttattttttttctctccgcctccccgtctcctcctcaggctcggaccatggtgcagtcccactggctcccctgcccccctctcctgtgagactggctgcggggagggatcatggatacttgtctgccggcttctggttcccacgcaagtaagcctgctgtcaatggaggaggacattgatacccgcaaaatcaacaacagtttcctgcgcgaccacagctatgcgaccgaagctgacattatctctacggtagaattcaaccacacgggagaattactagcgacaggggacaaggggggtcgggttgtaatatttcaacgagagcaggagagtaaaaatcaggttcatcgtaggggtgaatacaatgtttacagcacattccagagccatgaacccgagttcgattacctgaagagtttagaaatagaagaaaaaatcaataaaataagatggctcccccagcagaatgcagcttactttcttctgtctactaatgataaaactgtgaagctgtggaaagtcagcgagcgtgataagaggccagaaggctacaatctgaaagatgaggagggccggctccgggatcctgccaccatcacaaccctgcgggtgcctgtcctgagacccatggacctgatggtggaggccaccccacgaagagtatttgccaacgcacacacatatcacatcaactccatatctgtcaacagcgactatgaaacctacatgtccgctgatgacctgaggattaacctatggaactttgaaataaccaatcaaagttttaatattgtggacattaagccagccaacatggaggagctcacggaggtgatcacagcagccgagttccacccccatcattgcaacaccttcgtgtacagcagcagcaaagggacaatccggctgtgtgacatgcgggcatctgccctgtgtgacaggcacaccaaattttttgaagagccggaagatccaagcaacagatcatttttctctgaaattatctcttcgatttcggatgtgaagttcagccacagtgggaggtatatcatgaccagggactacttgaccgtcaaagtctgggatctcaacatggaaaaccgccccatcgagacttaccaggttcatgactacctccgcagcaagctgtgttccctctatgaaaatgactgcatttttgataaatttgagtgtgtgtggaatgggtcagacagtgtcatcatgacaggctcctacaacaacttcttcaggatgttcgacagaaacaccaagcgtgatgtgacccttgaggcttcgagggaaaacagcaagccccgggctatcctcaaaccccgaaaagtgtgtgtggggggcaagcggagaaaagacgagatcagtgtcgacagtctggactttagcaaaaagatcttgcatacagcttggcatccttcagaaaatattatagcagtggcggctacaaataacctatatatattccaggacaaggttaactaggtggacaagttattacttaataatctcacatactgaatactagtcaaacaagtttttaaatgtttctttgggtcttcatttgatgcattgactttaatttccctatacaggaaatgattggaatagaattaaaaggagtccaacattcccagctccccagttctaagaaacttttgtcaaacccaataggtttgggacacttctgtttagaattgaaagctgccagctaacagtaattcttccatagttgacttgaacttctgatgcttttattgcccagttttctctggtgggtccagtgttttgttcctaggtgtctgctgcgataaaatgaggttgtctgtagtatttaaggagaaaagagataagttttttttaattaagcaattccatttgattgaaaaaaatcaacaaaaaataaacaccgtttactcttagacaaa

SEQ ID NO: 87 - DNA, 1921 bp, Homo sapiens
  source 1..1921: mol_type = other DNA
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 88 - DNA, 10661 bp, Homo sapiens
  source 1..10661: mol_type = other DNA
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 89 - DNA, 4367 bp, Homo sapiens
  source 1..4367: mol_type = other DNA
gacgccatactggacgccaagtgggaggaacttcaaggctgtcccctgcgggcctcccgctctgcttctgcgaaggtttcattgaaaacagatcctgcaaaagttccaggtgcccacactggaaacttggagatcctgcttcccagaccacagctgtggggaacttggggtggagcagagaagtttctgtattcagctgcccaggcagaggagaatggggtctccacagcctgaagaatgaagacacgacagaataaagactcgatgtcaatgaggagtggacggaagaaagaggcccctgggccccgggaagaactgagatcgaggggccgggcctcccctggaggggtcagcacgtccagcagtgatggcaaagctgagaagtccaggcagacagccaagaaggcccgagtagaggaagcctccaccccaaaggtcaacaagcagggtcggagtgaggagatctcagagagtgaaagtgaggagaccaatgcaccaaaaaagaccaaaactgagcaggaactccctcggccacagtctccctccgatctggatagcttggacgggcggagccttaatgatgatggcagcagcgaccctagggatatcgaccaggacaaccgaagcacgtcccccagtatctacagccctggaagtgtggagaatgactctgactcatcttctggcctgtcccagggcccagcccgcccctaccacccacctccactctttcctccttcccctcaaccgccagacagcacccctcgacagccagaggctagctttgaaccccatccttctgtgacacccactggatatcatgctcccatggagccccccacatctcgaatgttccaggctcctcctggggcccctccccctcacccacagctctatcctgggggcactggtggagttttgtctggacccccaatgggtcccaaggggggaggggctgcctcatcagtggggggccctaatgggggtaagcagcaccccccacccactactcccatttcagtatcaagctctggggctagtggtgctcccccaacaaagccgcctaccactccagtgggtggtgggaacctaccttctgctccaccaccagccaacttcccccatgtgacaccgaacctgcctcccccacctgccctgagacccctcaacaatgcatcagcctctccccctggcctgggggcccaaccactacctggtcatctgccctctccccacgccatgggacagggtatgggtggacttcctcctggcccagagaagggcccaactctggctccttcaccccactctctgcctcctgcttcctcttctgctccagcgccccccatgaggtttccttattcatcctctagtagtagctctgcagcagcctcctcttccagttcttcctcctcttcctctgcctcccccttcccagcttcccaggcattgcccagctacccccactctttccctcccccaacaagcctctctgtctccaatcagccccccaagtatactcagccttctctcccatcccaggctgtgtggagccagggtcccccaccacctcctccctatggccgcctcttagccaacagcaatgcccatccaggccccttccctccctctactggggcccagtccaccgcccacccaccagtctcaacacatcaccatcaccaccagcaacagcaacagcagcagcagcagcagcagcagcagcagcagcagcagcagcagcatcacggaaactctgggccccctcctcctggagcatttccccacccactggagggcggtagctcccaccacgcacacccttacgccatgtctccctccctggggtctctgaggccctacccaccagggccagcacacctgcccccacctcacagccaggtgtcctacagccaagcaggccccaatggccctccagtctcttcctcttccaactcttcctcttccacttctcaagggtcctacccatgttcacacccctccccttcccagggccctcaaggggcgccctaccctttcccaccggtgcctacggtcaccacctcttcggctaccctttccacggtcattgccaccgtggcttcctcgccagcaggctacaaaacggcctccccacctgggcccccaccgtacggaaagagagccccgtccccgggggcctacaagacagccaccccacccggatacaaacccgggtcgcctccctccttccgaacggggaccccaccgggctatcgaggaacctcgccacctgcaggcccagggaccttcaagccgggctcgcccaccgtgggacctgggcccctgccacctgcggggccctcaggcctgccatcgctgccaccaccacctgcggcccctgcctcagggccgcccctgagcgccacgcagatcaaacaggagccggctgaggagtatgagacccccgagagcccggtgcccccagcccgcagcccctcgccccctcccaaggtggtagatgtacccagccatgccagtcagtctgccaggttcaacaaacacctggatcgcggcttcaactcgtgcgcgcgcagcgacctgtacttcgtgccactggagggctccaagctggccaagaagcgggccgacctggtggagaaggtgcggcgcgaggccgagcagcgcgcgcgcgaagaaaaggagcgcgagcgcgagcgggaacgcgagaaagagcgcgagcgcgagaaggagcgcgagcttgaacgcagcgtgaagttggctcaggagggccgtgctccggtggaatgcccatctctgggcccagtgccccatcgccctccatttgaaccgggcagtgcggtggctacagtgcccccctacctgggtcctgacactccagccttgcgcactctcagtgaatatgcccggcctcatgtcatgtctcctggcaatcgcaaccatccattctacgtgcccctgggggcagtggacccggggctcctgggttacaatgtcccggccctgtacagcagtgatccagctgcccgggagagggaacgggaagcccgtgaacgagacctccgtgaccgcctcaagcctggctttgaggtgaagcctagtgagctggaacccctacatggggtccctgggccgggcttggatccctttccccgacatgggggcctggctctgcagcctggcccacctggcctgcaccctttcccctttcatccgagcctggggcccctggagcgagaacgtctagcgctggcagctgggccagccctgcggcctgacatgtcctatgctgagcggctggcagctgagaggcagcacgcagaaagggtggcggccctgggcaatgacccactggcccggctgcagatgctcaatgtgactccccatcaccaccagcactcccacatccactcgcacctgcacctgcaccagcaagatgctatccatgcagcctctgcctcggtgcaccctctcattgaccccctggcctcagggtctcaccttacccggatcccctacccagctggaactctccctaaccccctgcttcctcaccctctgcacgagaacgaagttcttcgtcaccagctctttgctgccccttaccgggacctgccggcctccctttctgccccgatgtcagcagctcatcagctgcaggccatgcacgcacagtcagctgagctgcagcgcttggcgctggaacagcagcagtggctgcatgcccatcacccgctgcacagtgtgccgctgcctgcccaggaggactactacagtcacctgaagaaggaaagcgacaagccactgtagaacctgcgatcaagagagcaccatggctcctacattggaccttggagcacccccaccctccccccaccgtgcccttggcctgccacccagagccaagagggtgctgctcagttgcagggcctccgcagctggacagagagtgggggagggagggacagacagaaggccaaggcccgatgtggtgtgcagaggtggggaggtggcgaggatggggacagaaagcgcacagaatcttggaccaggtctctcttccttgtcccccctgcttttctcctcccccatgcccaacccctgtggccgccgcccctcccctgccccgttggtgtgattatttcatctgttagatgtggctgttttgcgtagcatcgtgtgccacccctgcccctccccgatccctgtgtgcgcgccccctctgcaatgtatgccccttgccccttccccacactaataatttatatatataaatatctatatgacgctcttaaaaaaacatcccaaccaaaaccaaccaaacaaaaacatcctcacaactccccaggaaaaaaaaaaaaaaaaaa

SEQ ID NO: 90 - RNA, 21 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  source 1..21: mol_type = other RNA; note = Oligonucleotide
ntgntgntgntgntgntgntg

SEQ ID NO: 91 - RNA, 21 nt, synthetic construct
  modified_base 1: mod_base = m5c
  source 1..21: mol_type = other RNA; note = Oligonucleotide
ntgctgctgctgctgctgctg

SEQ ID NO: 92 - RNA, 21 nt, synthetic construct
  modified_base 4: mod_base = m5c
  modified_base 10: mod_base = m5c
  modified_base 16: mod_base = m5c
  source 1..21: mol_type = other RNA; note = Oligonucleotide
ctgntgctgntgctgntgctg

SEQ ID NO: 93 - RNA, 21 nt, synthetic construct
  modified_base 1: mod_base = m5c
  modified_base 7: mod_base = m5c
  modified_base 13: mod_base = m5c
  modified_base 19: mod_base = m5c
  source 1..21: mol_type = other RNA; note = Oligonucleotide
ntgctgntgctgntgctgntg

SEQ ID NO: 94 - RNA, 21 nt, synthetic construct
  modified_base 7: mod_base = m5c
  modified_base 10: mod_base = m5c
  modified_base 13: mod_base = m5c
  source 1..21: mol_type = other RNA; note = Oligonucleotide
ctgctgntgntgntgctgctg

SEQ ID NO: 95 - RNA, 21 nt, synthetic construct
  modified_base 10: mod_base = m5c
  source 1..21: mol_type = other RNA; note = Oligonucleotide
ctgctgctgntgctgctgctg

SEQ ID NO: 96 - RNA, 21 nt, synthetic construct
  modified_base 1: mod_base = m5c
  modified_base 19: mod_base = m5c
  source 1..21: mol_type = other RNA; note = Oligonucleotide
ntgctgctgctgctgctgntg

SEQ ID NO: 97 - RNA, 21 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  source 1..21: mol_type = other RNA; note = Oligonucleotide
cngcngcngcngcngcngcng

SEQ ID NO: 98 - RNA, 21 nt, synthetic construct
  modified_base 2: mod_base = m5u
  source 1..21: mol_type = other RNA; note = Oligonucleotide
cngctgctgctgctgctgctg

SEQ ID NO: 99 - RNA, 21 nt, synthetic construct
  modified_base 5: mod_base = m5u
  modified_base 11: mod_base = m5u
  modified_base 17: mod_base = m5u
  source 1..21: mol_type = other RNA; note = Oligonucleotide
ctgcngctgcngctgcngctg

SEQ ID NO: 100 - RNA, 21 nt, synthetic construct
  modified_base 2: mod_base = m5u
  modified_base 8: mod_base = m5u
  modified_base 14: mod_base = m5u
  modified_base 20: mod_base = m5u
  source 1..21: mol_type = other RNA; note = Oligonucleotide
cngctgcngctgcngctgcng

SEQ ID NO: 101 - RNA, 21 nt, synthetic construct
  modified_base 8: mod_base = m5u
  modified_base 11: mod_base = m5u
  modified_base 14: mod_base = m5u
  source 1..21: mol_type = other RNA; note = Oligonucleotide
ctgctgcngcngcngctgctg

SEQ ID NO: 102 - RNA, 21 nt, synthetic construct
  modified_base 2: mod_base = m5u
  modified_base 20: mod_base = m5u
  source 1..21: mol_type = other RNA; note = Oligonucleotide
cngctgctgctgctgctgcng

SEQ ID NO: 103 - RNA, 21 nt, synthetic construct
  modified_base 8: mod_base = m5u
  modified_base 14: mod_base = m5u
  source 1..21: mol_type = other RNA; note = Oligonucleotide
ctgctgcngctgcngctgctg

SEQ ID NO: 104 - skipped

SEQ ID NO: 105 - RNA, 21 nt, synthetic construct
  modified_base 7: mod_base = m5c
  modified_base 8: mod_base = m5u
  modified_base 10: mod_base = m5c
  modified_base 11: mod_base = m5u
  modified_base 13: mod_base = m5c
  modified_base 14: mod_base = m5u
  source 1..21: mol_type = other RNA; note = Oligonucleotide
ctgctgnngnngnngctgctg

SEQ ID NO: 106 - RNA, 21 nt, synthetic construct
  modified_base 1: mod_base = m5c
  modified_base 2: mod_base = m5u
  modified_base 19: mod_base = m5c
  modified_base 20: mod_base = m5c
  source 1..21: mol_type = other RNA; note = Oligonucleotide
nngctgctgctgctgctgnng

SEQ ID NO: 107 - RNA, 21 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  source 1..21: mol_type = other RNA; note = Oligonucleotide
ntgntgntgntgntgntgntg

SEQ ID NO: 108 - RNA, 21 nt, synthetic construct
  modified_base 1: mod_base = m5c
  modified_base 7: mod_base = m5c
  modified_base 13: mod_base = m5c
  modified_base 19: mod_base = m5c
  source 1..21: mol_type = other RNA; note = Oligonucleotide
ntgctgntgctgntgctgntg

SEQ ID NO: 109 - RNA, 24 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  source 1..24: mol_type = other RNA; note = Oligonucleotide
ntgntgntgntgntgntgntgntg

SEQ ID NO: 110 - RNA, 24 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  modified_base 23: mod_base = m5u
  source 1..24: mol_type = other RNA; note = Oligonucleotide
cngcngcngcngcngcngcngcng

SEQ ID NO: 111 - RNA, 27 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  source 1..27: mol_type = other RNA; note = Oligonucleotide
ntgntgntgntgntgntgntgntgntg

SEQ ID NO: 112 - RNA, 27 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  modified_base 23: mod_base = m5u
  modified_base 26: mod_base = m5u
  source 1..27: mol_type = other RNA; note = Oligonucleotide
cngcngcngcngcngcngcngcngcng

SEQ ID NO: 113 - RNA, 30 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  modified_base 28: mod_base = m5c
  source 1..30: mol_type = other RNA; note = Oligonucleotide
ntgntgntgntgntgntgntgntgntgntg

SEQ ID NO: 114 - RNA, 30 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  modified_base 23: mod_base = m5u
  modified_base 26: mod_base = m5u
  modified_base 29: mod_base = m5u
  source 1..30: mol_type = other RNA; note = Oligonucleotide
cngcngcngcngcngcngcngcngcngcng

SEQ ID NO: 115 - RNA, 33 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  modified_base 28: mod_base = m5c
  modified_base 31: mod_base = m5c
  source 1..33: mol_type = other RNA; note = Oligonucleotide
ntgntgntgntgntgntgntgntgntgntgntg

SEQ ID NO: 116 - RNA, 33 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  modified_base 23: mod_base = m5u
  modified_base 26: mod_base = m5u
  modified_base 29: mod_base = m5u
  modified_base 32: mod_base = m5u
  source 1..33: mol_type = other RNA; note = Oligonucleotide
cngcngcngcngcngcngcngcngcngcngcng

SEQ ID NO: 117 - RNA, 36 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  modified_base 28: mod_base = m5c
  modified_base 31: mod_base = m5c
  modified_base 34: mod_base = m5c
  source 1..36: mol_type = other RNA; note = Oligonucleotide
ntgntgntgntgntgntgntgntgntgntgntgntg

SEQ ID NO: 118 - RNA, 36 nt, synthetic construct
  modified_base 2: mod_base = m5u
  modified_base 5: mod_base = m5u
  modified_base 8: mod_base = m5u
  modified_base 11: mod_base = m5u
  modified_base 14: mod_base = m5u
  modified_base 17: mod_base = m5u
  modified_base 20: mod_base = m5u
  modified_base 23: mod_base = m5u
  modified_base 26: mod_base = m5u
  modified_base 29: mod_base = m5u
  modified_base 32: mod_base = m5u
  modified_base 35: mod_base = m5u
  source 1..36: mol_type = other RNA; note = Oligonucleotide
cngcngcngcngcngcngcngcngcngcngcngcng

SEQ ID NO: 119-121 - skipped (3 entries)

SEQ ID NO: 122 - RNA, 15 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  source 1..15: mol_type = other RNA; note = Oligonucleotide
ngcngcngcngcngc

SEQ ID NO: 123 - skipped

SEQ ID NO: 124 - RNA, 18 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  source 1..18: mol_type = other RNA; note = Oligonucleotide
ngcngcngcngcngcngc

SEQ ID NO: 125 - skipped

SEQ ID NO: 126 - RNA, 21 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  source 1..21: mol_type = other RNA; note = Oligonucleotide
ngcngcngcngcngcngcngc

SEQ ID NO: 127 - skipped

SEQ ID NO: 128 - RNA, 24 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  source 1..24: mol_type = other RNA; note = Oligonucleotide
ngcngcngcngcngcngcngcngc

SEQ ID NO: 129 - skipped

SEQ ID NO: 130 - RNA, 27 nt, synthetic construct
  modified_base 1: mod_base = m5c
  variation 4: note = a, c, g, or u
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  source 1..27: mol_type = other RNA; note = Oligonucleotide
ngcngcngcngcngcngcngcngcngc

SEQ ID NO: 131 - RNA, 30 nt, synthetic construct; identical to SEQ ID NO: 19
  modified_base 1: mod_base = m5c
  modified_base 3: mod_base = m5c
  modified_base 4: mod_base = m5c
  modified_base 6: mod_base = m5c
  modified_base 7: mod_base = m5c
  modified_base 9: mod_base = m5c
  modified_base 10: mod_base = m5c
  modified_base 12: mod_base = m5c
  modified_base 13: mod_base = m5c
  modified_base 15: mod_base = m5c
  modified_base 16: mod_base = m5c
  modified_base 18: mod_base = m5c
  modified_base 19: mod_base = m5c
  modified_base 21: mod_base = m5c
  modified_base 22: mod_base = m5c
  modified_base 24: mod_base = m5c
  modified_base 25: mod_base = m5c
  modified_base 27: mod_base = m5c
  modified_base 28: mod_base = m5c
  modified_base 30: mod_base = m5c
  source 1..30: mol_type = other RNA; note = Oligonucleotide

SEQ ID NO: 132 - RNA, 30 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  modified_base 28: mod_base = m5c
  source 1..30: mol_type = other RNA; note = Oligonucleotide
ngcngcngcngcngcngcngcngcngcngc

SEQ ID NO: 133-135 - skipped (3 entries)

SEQ ID NO: 136 - RNA, 15 nt, synthetic construct
  modified_base 2: mod_base = m5c
  modified_base 5: mod_base = m5c
  modified_base 8: mod_base = m5c
  modified_base 11: mod_base = m5c
  modified_base 14: mod_base = m5c
  source 1..15: mol_type = other RNA; note = Oligonucleotide
cngcngcngcngcng

SEQ ID NO: 137 - skipped

SEQ ID NO: 138 - RNA, 18 nt, synthetic construct
  modified_base 2: mod_base = m5c
  modified_base 5: mod_base = m5c
  modified_base 8: mod_base = m5c
  modified_base 11: mod_base = m5c
  modified_base 14: mod_base = m5c
  modified_base 17: mod_base = m5c
  source 1..18: mol_type = other RNA; note = Oligonucleotide
cngcngcngcngcngcng

SEQ ID NO: 139 - skipped

SEQ ID NO: 140 - RNA, 21 nt, synthetic construct
  modified_base 2: mod_base = m5c
  modified_base 5: mod_base = m5c
  modified_base 8: mod_base = m5c
  modified_base 11: mod_base = m5c
  modified_base 14: mod_base = m5c
  modified_base 17: mod_base = m5c
  modified_base 20: mod_base = m5c
  source 1..21: mol_type = other RNA; note = Oligonucleotide
cngcngcngcngcngcngcng

SEQ ID NO: 141 - skipped

SEQ ID NO: 142 - RNA, 24 nt, synthetic construct
  modified_base 2: mod_base = m5c
  modified_base 5: mod_base = m5c
  modified_base 8: mod_base = m5c
  modified_base 11: mod_base = m5c
  modified_base 14: mod_base = m5c
  modified_base 17: mod_base = m5c
  modified_base 20: mod_base = m5c
  modified_base 23: mod_base = m5c
  source 1..24: mol_type = other RNA; note = Oligonucleotide
cngcngcngcngcngcngcngcng

SEQ ID NO: 143 - skipped

SEQ ID NO: 144 - RNA, 27 nt, synthetic construct
  modified_base 2: mod_base = m5c
  modified_base 5: mod_base = m5c
  modified_base 8: mod_base = m5c
  modified_base 11: mod_base = m5c
  modified_base 14: mod_base = m5c
  modified_base 17: mod_base = m5c
  variation 20: note = a, c, g, or u
  modified_base 23: mod_base = m5c
  modified_base 26: mod_base = m5c
  source 1..27: mol_type = other RNA; note = Oligonucleotide
cngcngcngcngcngcngcngcngcng

SEQ ID NO: 145 - RNA, 30 nt, synthetic construct; identical to SEQ ID NO: 10
  modified_base 1: mod_base = m5c
  modified_base 2: mod_base = m5c
  modified_base 4: mod_base = m5c
  modified_base 5: mod_base = m5c
  modified_base 7: mod_base = m5c
  modified_base 8: mod_base = m5c
  modified_base 10: mod_base = m5c
  modified_base 11: mod_base = m5c
  modified_base 13: mod_base = m5c
  modified_base 14: mod_base = m5c
  modified_base 16: mod_base = m5c
  modified_base 17: mod_base = m5c
  modified_base 19: mod_base = m5c
  modified_base 20: mod_base = m5c
  modified_base 22: mod_base = m5c
  modified_base 23: mod_base = m5c
  modified_base 25: mod_base = m5c
  modified_base 26: mod_base = m5c
  modified_base 28: mod_base = m5c
  modified_base 29: mod_base = m5c
  source 1..30: mol_type = other RNA; note = Oligonucleotide

SEQ ID NO: 146 - RNA, 30 nt, synthetic construct; identical to SEQ ID NO: 114
  modified_base 2: mod_base = m5c
  modified_base 5: mod_base = m5c
  modified_base 8: mod_base = m5c
  modified_base 11: mod_base = m5c
  modified_base 14: mod_base = m5c
  modified_base 17: mod_base = m5c
  modified_base 20: mod_base = m5c
  modified_base 23: mod_base = m5c
  modified_base 26: mod_base = m5c
  modified_base 29: mod_base = m5c
  source 1..30: mol_type = other RNA; note = Oligonucleotide

SEQ ID NO: 147-148 - skipped (2 entries)

SEQ ID NO: 149 - RNA, 15 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  source 1..15: mol_type = other RNA; note = Oligonucleotide
ttnttnttnttnttn

SEQ ID NO: 150 - skipped

SEQ ID NO: 151 - RNA, 18 nt, synthetic construct
  modified_base 3: mod_base = m5c
  modified_base 6: mod_base = m5c
  variation 9: note = a, c, g, or u
  modified_base 12: mod_base = m5c
  modified_base 15: mod_base = m5c
  modified_base 18: mod_base = m5c
  source 1..18: mol_type = other RNA; note = Oligonucleotide
ttnttnttnttnttnttn

SEQ ID NO: 152 - skipped

SEQ ID NO: 153 - RNA, 21 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  source 1..21: mol_type = other RNA; note = Oligonucleotide
ttnttnttnttnttnttnttn

SEQ ID NO: 154-155 - skipped (2 entries)

SEQ ID NO: 156 - RNA, 21 nt, synthetic construct
  modified_base 1: mod_base = m5u
  modified_base 2: mod_base = m5u
  modified_base 3: mod_base = m5c
  modified_base 19: mod_base = m5u
  modified_base 20: mod_base = m5u
  modified_base 21: mod_base = m5c
  source 1..21: mol_type = other RNA; note = Oligonucleotide
nnnttcttcttcttcttcnnn

SEQ ID NO: 157 - skipped

SEQ ID NO: 158 - RNA, 24 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  source 1..24: mol_type = other RNA; note = Oligonucleotide
ttnttnttnttnttnttnttnttn

SEQ ID NO: 159 - skipped

SEQ ID NO: 160 - RNA, 27 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  source 1..27: mol_type = other RNA; note = Oligonucleotide
ttnttnttnttnttnttnttnttnttn

SEQ ID NO: 161 - skipped

SEQ ID NO: 162 - RNA, 30 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  modified_base 30: mod_base = m5c
  source 1..30: mol_type = other RNA; note = Oligonucleotide
ttnttnttnttnttnttnttnttnttnttn

SEQ ID NO: 163 - RNA, 30 nt, synthetic construct
  modified_base 1: mod_base = m5u
  modified_base 2: mod_base = m5u
  modified_base 4: mod_base = m5u
  modified_base 5: mod_base = m5u
  modified_base 7: mod_base = m5u
  modified_base 8: mod_base = m5u
  modified_base 10: mod_base = m5u
  modified_base 11: mod_base = m5u
  modified_base 13: mod_base = m5u
  modified_base 14: mod_base = m5u
  modified_base 16: mod_base = m5u
  modified_base 17: mod_base = m5u
  modified_base 19: mod_base = m5u
  modified_base 20: mod_base = m5u
  modified_base 22: mod_base = m5u
  modified_base 23: mod_base = m5u
  modified_base 25: mod_base = m5u
  modified_base 26: mod_base = m5u
  modified_base 28: mod_base = m5u
  modified_base 29: mod_base = m5u
  source 1..30: mol_type = other RNA; note = Oligonucleotide
nncnncnncnncnncnncnncnncnncnnc

SEQ ID NO: 164 - RNA, 33 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  modified_base 30: mod_base = m5c
  modified_base 33: mod_base = m5c
  source 1..33: mol_type = other RNA; note = Oligonucleotide
ttnttnttnttnttnttnttnttnttnttnttn

SEQ ID NO: 165 - RNA, 33 nt, synthetic construct
  modified_base 1: mod_base = m5u
  modified_base 2: mod_base = m5u
  modified_base 4: mod_base = m5u
  modified_base 5: mod_base = m5u
  modified_base 7: mod_base = m5u
  modified_base 8: mod_base = m5u
  modified_base 10: mod_base = m5u
  modified_base 11: mod_base = m5u
  modified_base 13: mod_base = m5u
  modified_base 14: mod_base = m5u
  modified_base 16: mod_base = m5u
  modified_base 17: mod_base = m5u
  modified_base 19: mod_base = m5u
  modified_base 20: mod_base = m5u
  modified_base 22: mod_base = m5u
  modified_base 23: mod_base = m5u
  modified_base 25: mod_base = m5u
  modified_base 26: mod_base = m5u
  modified_base 28: mod_base = m5u
  modified_base 29: mod_base = m5u
  modified_base 31: mod_base = m5u
  modified_base 32: mod_base = m5u
  source 1..33: mol_type = other RNA; note = Oligonucleotide
nncnncnncnncnncnncnncnncnncnncnnc

SEQ ID NO: 166 - RNA, 36 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  modified_base 30: mod_base = m5c
  modified_base 33: mod_base = m5c
  modified_base 36: mod_base = m5c
  source 1..36: mol_type = other RNA; note = Oligonucleotide
ttnttnttnttnttnttnttnttnttnttnttnttn

SEQ ID NO: 167 - RNA, 36 nt, synthetic construct
  modified_base 1: mod_base = m5u
  modified_base 2: mod_base = m5u
  modified_base 4: mod_base = m5u
  modified_base 5: mod_base = m5u
  modified_base 7: mod_base = m5u
  modified_base 8: mod_base = m5u
  modified_base 10: mod_base = m5u
  modified_base 11: mod_base = m5u
  modified_base 13: mod_base = m5u
  modified_base 14: mod_base = m5u
  modified_base 16: mod_base = m5u
  modified_base 17: mod_base = m5u
  modified_base 19: mod_base = m5u
  modified_base 20: mod_base = m5u
  modified_base 22: mod_base = m5u
  modified_base 23: mod_base = m5u
  modified_base 25: mod_base = m5u
  modified_base 26: mod_base = m5u
  modified_base 28: mod_base = m5u
  modified_base 29: mod_base = m5u
  modified_base 31: mod_base = m5u
  modified_base 32: mod_base = m5u
  modified_base 34: mod_base = m5u
  modified_base 35: mod_base = m5u
  source 1..36: mol_type = other RNA; note = Oligonucleotide
nncnncnncnncnncnncnncnncnncnncnncnnc

SEQ ID NO: 168 - skipped

SEQ ID NO: 169 - RNA, 15 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  source 1..15: mol_type = other RNA; note = Oligonucleotide
ggnggnggnggnggn

SEQ ID NO: 170 - RNA, 18 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  source 1..18: mol_type = other RNA; note = Oligonucleotide
ggnggnggnggnggnggn

SEQ ID NO: 171 - RNA, 21 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  source 1..21: mol_type = other RNA; note = Oligonucleotide
ggnggnggnggnggnggnggn

SEQ ID NO: 172 - RNA, 21 nt, synthetic construct
  modified_base 3: mod_base = m5c
  modified_base 21: mod_base = m5c
  source 1..21: mol_type = other RNA; note = Oligonucleotide
ggnggcggcggcggcggcggn

SEQ ID NO: 173 - RNA, 21 nt, synthetic construct
  modified_base 3: mod_base = m5c
  modified_base 9: mod_base = m5c
  modified_base 15: mod_base = m5c
  modified_base 21: mod_base = m5c
  source 1..21: mol_type = other RNA; note = Oligonucleotide
ggnggcggnggcggnggcggn

SEQ ID NO: 174 - RNA, 21 nt, synthetic construct
  modified_base 9: mod_base = m5c
  modified_base 12: mod_base = m5c
  modified_base 15: mod_base = m5c
  source 1..21: mol_type = other RNA; note = Oligonucleotide
ggcggcggnggnggnggcggc

SEQ ID NO: 175 - RNA, 24 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  source 1..24: mol_type = other RNA; note = Oligonucleotide
ggnggnggnggnggnggnggnggn

SEQ ID NO: 176 - RNA, 27 nt, synthetic construct
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  source 1..27: mol_type = other RNA; note = Oligonucleotide
ggnggnggnggnggnggnggnggnggn

SEQ ID NO: 177 - RNA, 30 nt, synthetic construct; identical to SEQ ID NO: 46
  modified_base 3: mod_base = m5c
  modified_base 6: mod_base = m5c
  modified_base 9: mod_base = m5c
  modified_base 12: mod_base = m5c
  modified_base 15: mod_base = m5c
  modified_base 18: mod_base = m5c
  modified_base 21: mod_base = m5c
  modified_base 24: mod_base = m5c
  modified_base 27: mod_base = m5c
  modified_base 30: mod_base = m5c
  source 1..30: mol_type = other RNA; note = Oligonucleotide

SEQ ID NO: 178 - skipped

SEQ ID NO: 179 - RNA, 15 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  source 1..15: mol_type = other RNA; note = Oligonucleotide
nggnggnggnggngg

SEQ ID NO: 180 - RNA, 18 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  source 1..18: mol_type = other RNA; note = Oligonucleotide
nggnggnggnggnggngg

SEQ ID NO: 181 - RNA, 21 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  source 1..21: mol_type = other RNA; note = Oligonucleotide
nggnggnggnggnggnggngg

SEQ ID NO: 182 - RNA, 24 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  source 1..24: mol_type = other RNA; note = Oligonucleotide
nggnggnggnggnggnggnggngg

SEQ ID NO: 183 - RNA, 27 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  source 1..27: mol_type = other RNA; note = Oligonucleotide
nggnggnggnggnggnggnggnggngg

SEQ ID NO: 184 - RNA, 30 nt, synthetic construct; identical to SEQ ID NO: 55
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = m5c
  modified_base 25: mod_base = m5c
  modified_base 28: mod_base = m5c
  source 1..30: mol_type = other RNA; note = Oligonucleotide

SEQ ID NO: 185-186 - skipped (2 entries)

SEQ ID NO: 187 - RNA, 20 nt, synthetic construct
  misc_feature 1..20: note = Oligonucelotide
  modified_base 1: mod_base = OTHER; note = 2,6-diaminopurine
  modified_base 3: mod_base = OTHER; note = 2,6-diaminopurine
  modified_base 4: mod_base = OTHER; note = 2,6-diaminopurine
  modified_base 16: mod_base = OTHER; note = 2,6-diaminopurine
  modified_base 18: mod_base = OTHER; note = 2,6-diaminopurine
  modified_base 19: mod_base = OTHER; note = 2,6-diaminopurine
  source 1..20: mol_type = other RNA
ngnntagaatagaatngnnt

SEQ ID NO: 188 - RNA, 20 nt, synthetic construct
  modified_base 1: mod_base = OTHER; note = 2,6-diaminopurine
  modified_base 3: mod_base = OTHER; note = 2,6-diaminopurine
  modified_base 4: mod_base = OTHER; note = 2,6-diaminopurine
  modified_base 11: mod_base = OTHER; note = 2,6-diaminopurine
  modified_base 13: mod_base = OTHER; note = 2,6-diaminopurine
  modified_base 14: mod_base = OTHER; note = 2,6-diaminopurine
  source 1..20: mol_type = other RNA; note = Oligonucleotide
ngnntagaatngnntagaat

SEQ ID NO: 189 - RNA, 20 nt, synthetic construct
  modified_base 1: mod_base = OTHER; note = 2,6-diaminopurine
  modified_base 8: mod_base = OTHER; note = 2,6-diaminopurine
  modified_base 9: mod_base = OTHER; note = 2,6-diaminopurine
  modified_base 11: mod_base = OTHER; note = 2,6-diaminopurine
  modified_base 18: mod_base = OTHER; note = 2,6-diaminopurine
  modified_base 19: mod_base = OTHER; note = 2,6-diaminopurine
  source 1..20: mol_type = other RNA; note = Oligonucleotide
ngaatagnntngaatagnnt

SEQ ID NO: 190 - RNA, 25 nt, synthetic construct
  modified_base 1: mod_base = OTHER; note = 2,6-diaminopurine
  modified_base 3: mod_base = OTHER; note = 2,6-diaminopurine
  modified_base 4: mod_base = OTHER; note = 2,6-diaminopurine
  modified_base 6: mod_base = OTHER; note = 2,6-diaminopurine
  modified_base 8: mod_base = OTHER; note = 2,6-diaminopurine
  modified_base 9: mod_base = OTHER; note = 2,6-diaminopurine
  modified_base 11: mod_base = OTHER; note = 2,6-diaminopurine
  modified_base 13: mod_base = OTHER; note = 2,6-diaminopurine
  modified_base 14: mod_base = OTHER; note = 2,6-diaminopurine
  modified_base 16: mod_base = OTHER; note = 2,6-diaminopurine
  modified_base 18: mod_base = OTHER; note = 2,6-diaminopurine
  modified_base 19: mod_base = OTHER; note = 2,6-diaminopurine
  modified_base 21: mod_base = OTHER; note = 2,6-diaminopurine
  modified_base 23: mod_base = OTHER; note = 2,6-diaminopurine
  modified_base 24: mod_base = OTHER; note = 2,6-diaminopurine
  source 1..25: mol_type = other RNA; note = Oligonucleotide
ngnntngnntngnntngnntngnnt

SEQ ID NO: 191 - RNA, 30 nt, synthetic construct
  modified_base 1: mod_base = OTHER; note = 2,6-diaminopurine
  modified_base 3: mod_base = OTHER; note = 2,6-diaminopurine
  modified_base 4: mod_base = OTHER; note = 2,6-diaminopurine
  modified_base 6: mod_base = OTHER; note = 2,6-diaminopurine
  modified_base 8: mod_base = OTHER; note = 2,6-diaminopurine
  modified_base 9: mod_base = OTHER; note = 2,6-diaminopurine
  modified_base 11: mod_base = OTHER; note = 2,6-diaminopurine
  modified_base 13: mod_base = OTHER; note = 2,6-diaminopurine
  modified_base 14: mod_base = OTHER; note = 2,6-diaminopurine
  modified_base 16: mod_base = OTHER; note = 2,6-diaminopurine
  modified_base 18: mod_base = OTHER; note = 2,6-diaminopurine
  modified_base 19: mod_base = OTHER; note = 2,6-diaminopurine
  modified_base 21: mod_base = OTHER; note = 2,6-diaminopurine
  modified_base 23: mod_base = OTHER; note = 2,6-diaminopurine
  modified_base 24: mod_base = OTHER; note = 2,6-diaminopurine
  modified_base 26: mod_base = OTHER; note = 2,6-diaminopurine
  modified_base 28: mod_base = OTHER; note = 2,6-diaminopurine
  modified_base 29: mod_base = OTHER; note = 2,6-diaminopurine
  source 1..30: mol_type = other RNA; note = Oligonucleotide
ngnntngnntngnntngnntngnntngnnt

SEQ ID NO: 192 - RNA, 35 nt, synthetic construct
  modified_base 1: mod_base = OTHER; note = 2,6-diaminopurine
  modified_base 3: mod_base = OTHER; note = 2,6-diaminopurine
  modified_base 4: mod_base = OTHER; note = 2,6-diaminopurine
  modified_base 6: mod_base = OTHER; note = 2,6-diaminopurine
  modified_base 8: mod_base = OTHER; note = 2,6-diaminopurine
  modified_base 9: mod_base = OTHER; note = 2,6-diaminopurine
  modified_base 11: mod_base = OTHER; note = 2,6-diaminopurine
  modified_base 13: mod_base = OTHER; note = 2,6-diaminopurine
  modified_base 14: mod_base = OTHER; note = 2,6-diaminopurine
  modified_base 16: mod_base = OTHER; note = 2,6-diaminopurine
  modified_base 18: mod_base = OTHER; note = 2,6-diaminopurine
  modified_base 19: mod_base = OTHER; note = 2,6-diaminopurine
  modified_base 21: mod_base = OTHER; note = 2,6-diaminopurine
  modified_base 23: mod_base = OTHER; note = 2,6-diaminopurine
  modified_base 24: mod_base = OTHER; note = 2,6-diaminopurine
  modified_base 26: mod_base = OTHER; note = 2,6-diaminopurine
  modified_base 28: mod_base = OTHER; note = 2,6-diaminopurine
  modified_base 29: mod_base = OTHER; note = 2,6-diaminopurine
  modified_base 31: mod_base = OTHER; note = 2,6-diaminopurine
  modified_base 33: mod_base = OTHER; note = 2,6-diaminopurine
  modified_base 34: mod_base = OTHER; note = 2,6-diaminopurine
  source 1..35: mol_type = other RNA; note = Oligonucleotide
ngnntngnntngnntngnntngnntngnntngnnt

SEQ ID NO: 193-194 - skipped (2 entries)

SEQ ID NO: 195 - RNA, 16 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  source 1..16: mol_type = other RNA; note = Oligonucleotide
cnggcnggcnggcngg

SEQ ID NO: 196 - RNA, 16 nt, synthetic construct
  modified_base 1: mod_base = m5c
  modified_base 5: mod_base = m5c
  modified_base 9: mod_base = m5c
  modified_base 13: mod_base = m5c
  source 1..16: mol_type = other RNA; note = Oligonucleotide
naggnaggnaggnagg

SEQ ID NO: 197 - RNA, 20 nt, synthetic construct
  modified_base 1: mod_base = m5c
  modified_base 5: mod_base = m5c
  modified_base 9: mod_base = m5c
  modified_base 13: mod_base = m5c
  modified_base 17: mod_base = m5c
  source 1..20: mol_type = other RNA; note = Oligonucleotide
naggnaggnaggnaggnagg

SEQ ID NO: 198 - RNA, 20 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  modified_base 18: mod_base = OTHER; note = 2,6-diaminopurine
  source 1..20: mol_type = other RNA; note = Oligonucleotide
cnggcnggcnggcnggcngg

SEQ ID NO: 199 - RNA, 20 nt, synthetic construct
  modified_base 1: mod_base = m5c
  modified_base 9: mod_base = m5c
  modified_base 17: mod_base = m5c
  source 1..20: mol_type = other RNA; note = Oligonucleotide
naggcaggnaggcaggnagg

SEQ ID NO: 200 - RNA, 20 nt, synthetic construct
  modified_base 2: mod_base = OTHER; note = 2,6-diaminopurine
  modified_base 10: mod_base = OTHER; note = 2,6-diaminopurine
  modified_base 18: mod_base = OTHER; note = 2,6-diaminopurine
  source 1..20: mol_type = other RNA; note = Oligonucleotide
cnggcaggcnggcaggcngg

SEQ ID NO: 201 - RNA, 24 nt, synthetic construct
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  source 1..24: mol_type = other RNA; note = Oligonucleotide
naggnaggnaggnaggnaggnagg

SEQ ID NO: 202 - RNA, 24 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  modified_base 18: mod_base = OTHER; note = 2,6-diaminopurine
  modified_base 22: mod_base = OTHER; note = 2,6-diaminopurine
  source 1..24: mol_type = other RNA; note = Oligonucleotide
cnggcnggcnggcnggcnggcngg

SEQ ID NO: 203 - RNA, 28 nt, synthetic construct
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  source 1..28: mol_type = other RNA; note = Oligonucleotide
naggnaggnaggnaggnaggnaggnagg

SEQ ID NO: 204 - RNA, 28 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  modified_base 18: mod_base = OTHER; note = 2,6-diaminopurine
  modified_base 22: mod_base = OTHER; note = 2,6-diaminopurine
  modified_base 26: mod_base = OTHER; note = 2,6-diaminopurine
  source 1..28: mol_type = other RNA; note = Oligonucleotide
cnggcnggcnggcnggcnggcnggcngg

SEQ ID NO: 205 - RNA, 32 nt, synthetic construct
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  modified_base 29: mod_base = m5c
  source 1..32: mol_type = other RNA; note = Oligonucleotide
naggnaggnaggnaggnaggnaggnaggnagg

SEQ ID NO: 206 - RNA, 32 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  modified_base 18: mod_base = OTHER; note = 2,6-diaminopurine
  modified_base 22: mod_base = OTHER; note = 2,6-diaminopurine
  modified_base 26: mod_base = OTHER; note = 2,6-diaminopurine
  modified_base 30: mod_base = OTHER; note = 2,6-diaminopurine
  source 1..32: mol_type = other RNA; note = Oligonucleotide
cnggcnggcnggcnggcnggcnggcnggcngg

SEQ ID NO: 207 - RNA, 36 nt, synthetic construct
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  modified_base 29: mod_base = m5c
  modified_base 33: mod_base = m5c
  source 1..36: mol_type = other RNA; note = Oligonucleotide
naggnaggnaggnaggnaggnaggnaggnaggnagg

SEQ ID NO: 208 - RNA, 36 nt, synthetic construct
  modified_base 2: mod_base = OTHER; note = 2,6-diaminopurine
  modified_base 6: mod_base = OTHER; note = 2,6-diaminopurine
  modified_base 10: mod_base = OTHER; note = 2,6-diaminopurine
  modified_base 14: mod_base = OTHER; note = 2,6-diaminopurine
  modified_base 18: mod_base = OTHER; note = 2,6-diaminopurine
  modified_base 22: mod_base = OTHER; note = 2,6-diaminopurine
  modified_base 26: mod_base = OTHER; note = 2,6-diaminopurine
  modified_base 30: mod_base = OTHER; note = 2,6-diaminopurine
  modified_base 34: mod_base = OTHER; note = 2,6-diaminopurine
  source 1..36: mol_type = other RNA; note = Oligonucleotide
cnggcnggcnggcnggcnggcnggcnggcnggcngg

SEQ ID NO: 209 - RNA, 17 nt, synthetic construct
  modified_base 6: mod_base = m5c
  modified_base 12: mod_base = m5c
  source 1..17: mol_type = other RNA; note = Oligonucleotide
ggctcnggctcnggctc

SEQ ID NO: 210 - DNA, 18 bp, synthetic construct
  modified_base 4: mod_base = OTHER; note = abasic monomer
  modified_base 6: mod_base = m5c
  modified_base 10: mod_base = OTHER; note = abasic monomer
  modified_base 12: mod_base = m5c
  modified_base 16: mod_base = OTHER; note = abasic monomer
  modified_base 18: mod_base = m5c
  source 1..18: mol_type = other DNA; note = Oligonucleotide
ggcncnggcncnggcncn

SEQ ID NO: 211 - RNA, 18 nt, synthetic construct
  modified_base 6: mod_base = m5c
  modified_base 12: mod_base = m5c
  modified_base 18: mod_base = m5c
  source 1..18: mol_type = other RNA; note = Oligonucleotide
ggctcnggctcnggctcn

SEQ ID NO: 212 - RNA, 24 nt, synthetic construct
  modified_base 6: mod_base = m5c
  modified_base 12: mod_base = m5c
  modified_base 18: mod_base = m5c
  modified_base 24: mod_base = m5c
  source 1..24: mol_type = other RNA; note = Oligonucleotide
ggctcnggctcnggctcnggctcn

SEQ ID NO: 213 - RNA, 24 nt, synthetic construct
  modified_base 4: mod_base = OTHER; note = abasic monomer
  modified_base 6: mod_base = m5c
  modified_base 10: mod_base = OTHER; note = abasic monomer
  modified_base 12: mod_base = m5c
  modified_base 16: mod_base = OTHER; note = abasic monomer
  modified_base 18: mod_base = m5c
  modified_base 22: mod_base = OTHER; note = abasic monomer
  modified_base 24: mod_base = m5c
  source 1..24: mol_type = other RNA; note = Oligonucleotide
ggcncnggcncnggcncnggcncn

SEQ ID NO: 214 - RNA, 24 nt, synthetic construct
  modified_base 4: mod_base = i
  modified_base 6: mod_base = m5c
  modified_base 10: mod_base = i
  modified_base 12: mod_base = m5c
  modified_base 16: mod_base = i
  modified_base 18: mod_base = m5c
  modified_base 22: mod_base = i
  modified_base 24: mod_base = m5c
  source 1..24: mol_type = other RNA; note = Oligonucleotide
ggcncnggcncnggcncnggcncn

SEQ ID NO: 215 - RNA, 24 nt, synthetic construct
  modified_base 6: mod_base = m5c
  modified_base 12: mod_base = m5c
  modified_base 18: mod_base = m5c
  modified_base 24: mod_base = m5c
  source 1..24: mol_type = other RNA; note = Oligonucleotide
ggcctnggcctnggcctnggcctn

SEQ ID NO: 216 - skipped

SEQ ID NO: 217 - RNA, 24 nt, synthetic construct
  variation 3: note = c or m5c
  variation 5: note = c or m5c
  variation 6: note = c or m5c
  variation 9: note = c or m5c
  variation 11: note = c or m5c
  variation 12: note = c or m5c
  variation 15: note = c or m5c
  variation 17: note = c or m5c
  variation 18: note = c or m5c
  variation 21: note = c or m5c
  variation 23: note = c or m5c
  variation 24: note = c or m5c
  source 1..24: mol_type = other RNA; note = Oligonucleotide
ggntnnggntnnggntnnggntnn

SEQ ID NO: 218-219 - skipped (2 entries)

SEQ ID NO: 220 - RNA, 25 nt, synthetic construct
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  modified_base 22: mod_base = OTHER; note = abasic monomer
  modified_base 23: mod_base = OTHER; note = abasic monomer
  modified_base 24: mod_base = OTHER; note = abasic monomer
  modified_base 25: mod_base = OTHER; note = abasic monomer
  source 1..25: mol_type = other RNA; note = Oligonucleotide
ntgntgntgntgntgntgntgnnnn

SEQ ID NO: 221 - RNA, 25 nt, synthetic construct
  modified_base 1: mod_base = m5c
  modified_base 7: mod_base = m5c
  modified_base 13: mod_base = m5c
  modified_base 19: mod_base = m5c
  modified_base 22: mod_base = OTHER; note = abasic monomer
  modified_base 23: mod_base = OTHER; note = abasic monomer
  modified_base 24: mod_base = OTHER; note = abasic monomer
  modified_base 25: mod_base = OTHER; note = abasic monomer
  source 1..25: mol_type = other RNA; note = Oligonucleotide
ntgctgntgctgntgctgntgnnnn